(19) United States
(12) Patent Application Publication
(10) Pub. No.: US 2018/0319753 A1
(43) Pub. Date: Nov. 8, 2018

(54) MICROBIOCIDAL OXADIAZOLE DERIVATIVES

(71) Applicant: SYNGENTA PARTICIPATIONS AG, Basel (CH)

(72) Inventors: Thomas James Hoffman, Stein (CH); Daniel Stierli, Stein (CH); Martin Pouliot, Stein (CH); Renaud Beaudegnies, Stein (CH)

(73) Assignee: SYNGENTA PARTICIPATIONS AG, Basel (CH)

(21) Appl. No.: 15/771,299

(22) PCT Filed: Oct. 27, 2016
(86) PCT No.: PCT/EP2016/075961

§ 371 Date: Apr. 30, 2018

(30) Foreign Application Priority Data
Oct. 28, 2015 (EP) 15191906.5

(51) Int. Cl. C07D 271/06; C07D 413/04; A01N 43/82

(52) U.S. Cl. CPC C07D 271/06; A01N 43/82; C07D 413/04

(57) ABSTRACT

Compounds of the formula (I) wherein the substituents are as defined in claim 1, useful as a pesticides, especially as fungicides.

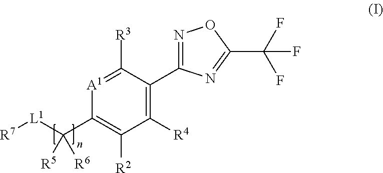

The present invention relates to microbiocidal oxadiazole derivatives, eg, as active ingredients, which have microbiocidal activity, in particular, fungicidal activity. The invention also relates to agrochemical compositions which comprise at least one of the oxadiazole derivatives, to processes of preparation of these compounds and to uses of the oxadiazole derivatives or compositions in agriculture or horticulture for controlling or preventing infestation of plants, harvested food crops, seeds or non-living materials by phytopathogenic microorganisms, preferably fungi.

WO 94/05153 discloses herbicidal compositions comprising halo-substituted benzene compounds for control of the growth of undesired vegetation.

According to the present invention, there is provided compound of formula (I):

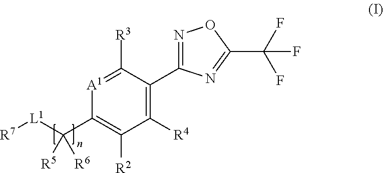

wherein

A¹ represents N or CR¹, wherein R¹ is selected from hydrogen, halogen, methyl, trifluoromethyl or methoxy;

R² is hydrogen or halogen;

R³ and R⁴ are independently selected from hydrogen and fluorine; and wherein at least two of R¹ to R⁴ are hydrogen;

n represents 0, 1 or 2;

R⁵ and R⁶ are independently selected from hydrogen, C₁₋₄alkyl and cyano;

L¹ represents S, S(O) or S(O)₂;

R⁷ represents hydrogen, C₁₋₆alkyl, C₂₋₆alkenyl, C₂₋₆alkynyl, cyanoC₁₋₆alkyl, C₁₋₆haloalkyl, C₂₋₆haloalkenyl, hydroxyC₁₋₆alkyl, C₁₋₄alkoxyC₁₋₆alkyl, C₁₋₄alkoxyC₁₋₆alkoxy or C₁₋₄haloalkoxyC₁₋₆alkyl; or

R⁷ represents C₃₋₈cycloalkyl or C₃₋₈cycloalkylC₁₋₃alkyl wherein the cycloalkyl moiety is optionally partially unsaturated, phenyl, phenylC₁₋₃alkyl, heteroaryl bonded to L¹ through a carbon atom or heteroarylC₁₋₃alkyl wherein the heteroaryl moiety is a 5- or 6-membered monocyclic aromatic ring which comprises 1, 2, 3 or 4 heteroatoms individually selected from N, O and S, heterocyclyl bonded to L¹ through a carbon atom or heterocyclylC₁₋₃alkyl wherein the heterocyclyl moiety is a 4- to 6-membered non-aromatic ring which comprises 1, 2 or 3 heteroatoms individually selected from N, O and S, and wherein C₃₋₈cycloalkyl, C₃₋₈cycloalkylC₁₋₃alkyl, phenyl, phenylC₁₋₃alkyl, heteroaryl, heteroarylC₁₋₃alkyl, heterocyclyl and heterocyclylC₁₋₃alkyl are optionally substituted by 1, 2, 3, 4 or 5 substituents, which may be the same or different, selected from R⁸;

R⁸ represents cyano, halogen, hydroxy, C₁₋₄alkyl, C₁₋₄haloalkyl, C₁₋₄alkoxy or C₁₋₄haloalkoxy; and

wherein when R⁷ represents C₃₋₈cycloalkyl, C₃₋₈cycloalkylC₁₋₃alkyl, heterocyclyl or heterocyclylC₁₋₃alkyl, the C₃₋₈cycloalkyl moiety or the heterocyclyl moiety is optionally substituted by 1 or 2 oxo groups; or

a salt or an N-oxide thereof.

Surprisingly, it has been found that the novel compounds of formula (I) have, for practical purposes, a very advantageous level of biological activity for protecting plants against diseases that are caused by fungi.

According to a second aspect of the invention, there is provided an agrochemical composition comprising a fungicidally effective amount of a compound of formula (I).

According to a third aspect of the invention, there is provided a method of controlling or preventing infestation of useful plants by phytopathogenic microorganisms, wherein a fungicidally effective amount of a compound of formula (I), or a composition comprising this compound as active ingredient, is applied to the plants, to parts thereof or the locus thereof.

According to a fourth aspect of the invention, there is provided the use of a compound of formula (I) as a fungicide. According to this particular aspect of the invention, the use may or may not include methods for the treatment of the human or animal body by surgery or therapy.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, in the definition of L¹, S means a sulfanyl group, S(O) means a sulfinyl group and S(O)₂ means a sulfonyl group.

As used herein, oxo means an ═O group, eg, as present in a ketonyl (—C(═O)—) group.

As used herein, the term “C₁₋₆alkyl” refers to a straight or branched hydrocarbon chain radical consisting solely of carbon and hydrogen atoms, containing no unsaturation, having from one to six carbon atoms, and which is attached to the rest of the molecule by a single bond. The terms “C₁₋₂alkyl”, “C₁₋₃alkyl” and “C₁₋₄alkyl” are to be construed accordingly. Examples of C₁₋₆alkyl include, but are not limited to, methyl, ethyl, n-propyl, 1-methylethyl (iso-propyl), n-butyl,1-dimethylethyl (t-butyl) and n-pentyl. A “C₁₋₃alkylene” group refers to the corresponding definition of C₁₋₃alkyl (and C₁₋₂alkyl), except that such radical is attached to the rest of the molecule by two single bonds. Examples of C₁₋₃alkylene, include, but are not limited to, —CH₂—, —CH₂CH₂— and —(CH₂)₃—.

As used herein, the term “C₂₋₆ alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. The term “C₂₋₄ alkenyl” is to be construed accordingly. Examples of C₂₋₆alkenyl include, but are not limited to, ethenyl, prop-1-enyl, but-1-enyl.

As used herein, the term “C₂₋₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. The term “C₂₋₄alkynyl” is to be construed accordingly. Examples of C₂₋₆alkynyl include, but are not limited to, ethynyl, prop-1-ynyl, but-1-ynyl.

As used herein, the term “cyanoC₁₋₆alkyl” refers to a C₁₋₆alkyl radical as generally defined above substituted by one or more cyano groups as defined above. The term “cyanoC₁₋₄alkyl” is to be construed accordingly. Examples of cyanoC₁₋₆alkyl include, but are not limited to cyanomethyl, cyanoethyl.

As used herein, the term “C₁₋₆haloalkyl” refers to a C₁₋₆alkyl radical as generally defined above substituted by one or more of the same or different halogen atoms. The term “C₁₋₄haloalkyl” is to be construed accordingly. Examples of C₁₋₆haloalkyl include, but are not limited to fluoromethyl, difluoromethyl, fluoroethyl, trifluoromethyl, 2,2,2-trifluoroethyl.

As used herein, the term “C₂₋₆haloalkenyl” refers to a C₂₋₆alkenyl radical as generally defined above substituted by one or more of the same or different halogen atoms. The term “C₂₋₄haloalkenyl” is to be construed accordingly.

As used herein, the term “hydroxyC₁₋₆alkyl” refers to a C₁₋₆alkyl radical as generally defined above substituted by one or more hydroxy groups as defined above. The term “hydroxyC₁₋₄alkyl” is to be construed accordingly.

As used herein, the term “C₁₋₆alkoxy” refers to a radical of the formula —ORₐ where Rₐ is a C₁₋₆ alkyl radical as generally defined above. The term “C₁₋₄alkoxy” is to be construed accordingly. Examples of C₁₋₆alkoxy include, but are not limited to, methoxy, ethoxy, propoxy, iso-propoxy, butoxy. As used herein, the term “C₁₋₄alkoxyC₁₋₆alkyl” refers to a C₁₋₆alkyl radical as generally defined above substituted by a C₁₋₄alkoxy group as defined above. The term “C₁₋₄alkoxyC₁₋C₄alkyl” is to be construed accordingly. Examples of C₁₋₄alkoxyC₁₋₆alkyl include, but are not limited to methoxymethyl, 2-methoxyethyl.

As used herein, the term “C₁₋C₄alkoxyC₁₋C₆alkoxy” refers to a C₁₋₆alkoxy radical as generally defined above substituted by a C₁₋₄alkoxy group as defined above. The term “C₁₋C₄alkoxyC₁₋C₄alkoxy” is to be construed accordingly.

As used herein, the term “C₁₋₄haloalkoxy” refers to a C₁₋₄alkoxy radical as generally defined above substituted by one or more halogen atoms as defined above.

As used herein, the term “C₁₋₄haloalkoxyC₁₋₆alkyl” refers to a C₁₋₆alkyl radical as generally defined above substituted by one or more C₁₋₄haloalkoxy groups as defined above. The term “C₁₋₄halolkoxyC₁₋₄alkyl” is to be construed accordingly.

As used herein, the term “C₃₋₈cycloalkyl” refers to a mono- or bi-cyclic ring system containing 3 to 8 carbon atoms. The terms “C₃₋₆cycloalkyl” and “C₃₋₅cycloalkyl” are to be construed accordingly. Examples of C₃₋₈cycloalkyl include, but are not limited to cyclopropyl, methylcyclopropyl, cyclobutyl, cyclopentyl and cyclohexyl.

As used herein, the term “heteroaryl” refers to a 5- or 6-membered aromatic monocyclic ring radical which comprises 1, 2, 3 or 4 heteroatoms individually selected from nitrogen, oxygen and sulfur. Examples of heteroaryl include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl.

As used herein, the term “heterocyclyl” or “heterocyclic” refers to a stable 4-, 5- or 6-membered non-aromatic monocyclic ring radical which comprises 1, 2, or 3, heteroatoms individually selected from nitrogen, oxygen and sulfur. Examples of heterocyclyl include, but are not limited to azetidinyl, oxetanyl, pyrrolinyl, pyrrolidinyl, thietanyl, tetrahydrofuryl, tetrahydrothienyl, tetrahydrothiopyranyl, piperidinyl, piperazinyl, tetrahydropyranyl, morpholinyl, perhydroazepinyl.

As used herein, the term “C₃₋₈cycloalkylC₁₋₃alkyl” refers to a C₃₋₈cycloalkyl ring as generally defined above attached to the rest of the molecule by a C₁₋₃alkylene radical as generally defined above. The term “C₃₋₈ cycloalkylC₁₋₂alkyl” is to be construed accordingly. Examples of C₃₋₈cycloalkylC₁₋₃alkyl include, but are not limited to cyclopropylmethyl or cyclopropylethyl.

As used herein, the term “phenylC₁₋₃alkyl” refers to a phenyl ring attached to the rest of the molecule by a C₁₋₃alkylene radical as generally defined above. The term “phenylC₁₋₂alkyl” is to be construed accordingly. Examples of phenylC₁₋₃alkyl include, but are not limited to benzyl or 2-phenylethyl.

As used herein, the term “heteroarylC₁₋₃alkyl” refers to a heteroaryl ring as generally defined above attached to the rest of the molecule by a C₁₋₃alkylene radical as generally defined above.

As used herein, the term “heterocyclylC₁₋₃alkyl” refers to a heterocyclyl ring as generally defined above attached to the rest of the molecule by a C₁₋₃alkylene radical as generally defined above.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I).

In each case, the compounds of formula (I) according to the invention are in free form, in covalently hydrated form, in oxidized form as an N-oxide or in salt form, e.g., an agronomically usable or agrochemically acceptable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The following list provides definitions, including preferred definitions, for substituents A¹, R¹, R², R³, R⁴, R⁵, R⁶, R⁷, R⁸, n and L¹ with reference to the compounds of formula (I). For any one of these substituents, any of the definitions given below may be combined with any definition of any other substituent given below or elsewhere in this document.

A¹ represents N or CR¹, wherein R¹ is selected from hydrogen, halogen, methyl, trifluoromethyl or methoxy. Preferably, A¹ represents N or CR¹, wherein R¹ is selected from hydrogen or methyl. More preferably, A¹ is CR¹, wherein R¹ is hydrogen.

R² is hydrogen or halogen. Preferably, R² is hydrogen or fluorine, in particular, hydrogen.

R³ and R⁴ are independently selected from hydrogen and fluorine.

Preferably, R², R³ and R⁴ are independently selected from hydrogen and fluorine. More preferably, R², R³ and R⁴ are hydrogen.

In the compounds according to formula (I), at least two of R¹ to R⁴ are hydrogen.

In some embodiments of the invention, the 6-membered ring comprising A¹ is a phenyl (where A¹ is C—H and R², R³ and R⁴ are all hydrogen), a pyridinyl (where A¹ is N and R², R³ and R⁴ are all hydrogen), a fluorophenyl (where A¹ is C—F or R³ is fluoro, and the other ring positions are C—H) or a difluorophenyl (where A¹ is C—F and R³ is fluoro, or A¹ is C—F and R² is fluoro, and the other ring positions are C—H) group.

n represents 0, 1 or 2. In one embodiment of the invention, n is 0. In another embodiment of the invention, n is 1. In yet another embodiment of the invention, n is 2. Preferably, n represents 0 or 1, and most preferably n represents 0.

R⁵ and R⁶ are independently selected from hydrogen, C₁₋₄alkyl and cyano. Preferably, R⁵ and R⁶ are hydrogen or R⁵ is hydrogen and R⁶ is methyl. Most preferably, R⁵ and R⁶ are hydrogen.

L¹ represents S, S(O) or S(O)₂. In one embodiment of the invention, L¹ is S. In another embodiment of the invention, L¹ is S(O). In yet another embodiment of the invention, L¹ is S(O)₂. Preferably, L¹ represents S or S(O).

R⁷ represents C₃₋₈cycloalkyl or C₃₋₈cycloalkylC₁₋₃alkyl wherein the cycloalkyl moiety is optionally partially unsaturated, phenyl, phenylC₁₋₃alkyl, heteroaryl bonded to L¹ through a carbon atom or heteroarylC₁₋₃alkyl wherein the heteroaryl moiety is a 5- or 6-membered monocyclic aromatic ring which comprises 1, 2, 3 or 4 heteroatoms individually selected from N, O and S, heterocyclyl bonded to L¹ through a carbon atom or heterocyclylC₁₋₃alkyl wherein the heterocyclyl moiety is a 4- to 6-membered non-aromatic ring which comprises 1, 2 or 3 heteroatoms individually selected from N, O and S, and wherein C₃₋₈cycloalkyl, C₃₋₈cycloalkylC₁₋₃alkyl, phenyl, phenylC₁₋₃alkyl, heteroaryl, heteroarylC₁₋₃alkyl, heterocyclyl and heterocyclylC₁₋₃alkyl are optionally substituted by 1, 2, 3, 4 or 5 substituents, which may be the same or different, selected from R⁸; wherein R⁸ represents cyano, halogen, hydroxy, C₁₋₄alkoxy or C₁₋₄haloalkoxy; and wherein when R⁷ represents C₃₋₈cycloalkyl, C₃₋₈cycloalkylC₁₋₃alkyl, heterocyclyl or heterocyclylC₁₋₃alkyl, the C₃₋₈cycloalkyl moiety or the heterocyclyl moiety is optionally substituted by 1 or 2 oxo groups.

Preferably, R⁷ represents hydrogen, C₁₋₈alkyl, C₂₋₈alkenyl, C₂₋₈alkynyl, cyanoC₁₋₈alkyl, C₁₋₈haloalkyl or C₁₋₄alkoxyC₁₋₈alkyl; or C₃₋₈cycloalkyl or C₃₋₈cycloalkylC₁₋₂alkyl wherein the cycloalkyl moiety is optionally partially unsaturated, phenyl or phenylC₁₋₂alkyl, heteroaryl bonded to L¹ through a carbon atom or heteroarylC₁₋₂alkyl wherein the heteroaryl moiety is a 5- or 6-membered monocyclic aromatic ring which comprises 1, 2, 3 or 4 heteroatoms individually selected from N, O and S, or heterocyclyl bonded to L¹ through a carbon atom or heterocyclylC₁₋₂alkyl wherein the heterocyclyl moiety is a 4- to 6-membered non-aromatic ring which comprises 1, 2 or 3 heteroatoms individually selected from N, O and S; wherein any C₃₋₈cycloalkyl or C₃₋₈cycloalkylC₁₋₂alkyl, phenyl or phenylC₁₋₂alkyl, heteroaryl or heteroarylC₁₋₂alkyl, or heterocyclyl or heterocyclylC₁₋₂alkyl moiety is optionally substituted by 1, 2, or 3 substituents, which may be the same or different, selected from R⁸; wherein R⁸ represents halogen, C₁₋₄alkyl, C₁₋₄haloalkyl and C₁₋₄alkoxy.

Alternatively, R⁷ represents hydrogen, C₁₋₈alkyl, C₂₋₈alkenyl, C₂₋₈alkynyl, cyanoC₁₋₈alkyl, C₁₋₈haloalkyl or C₁₋₄alkoxyC₁₋₈alkyl; or C₃₋₈cycloalkyl or C₃₋₈cycloalkylC₁₋₂alkyl wherein the cycloalkyl moiety is optionally partially unsaturated, phenyl or phenylC₁₋₂alkyl, heteroaryl bonded to L¹ through a carbon atom or heteroarylC₁₋₂alkyl wherein the heteroaryl moiety is a 5- or 6-membered monocyclic aromatic ring which comprises 1, 2, 3 or 4 heteroatoms individually selected from N, O and S, or heterocyclyl bonded to L¹ through a carbon atom or heterocyclylC₁₋₂alkyl wherein the heterocyclyl moiety is a 4- to 6-membered non-aromatic ring which comprises 1, 2 or 3 heteroatoms individually selected from N, O and S; wherein any cycloalkyl, phenyl, heteroaryl or moiety is optionally substituted by 1, 2, or 3 substituents, which may be the same or different, selected from R⁸; wherein R⁸ represents halogen, C₁₋₄alkyl, C₁₋₄haloalkyl and C₁₋₄alkoxy.

More preferably, R⁷ represents C₁₋₆alkyl, C₂₋₆alkenyl, C₂₋₆alkynyl, C₁₋₆haloalkyl or C₁₋₄alkoxyC₁₋₆alkyl; or C₃₋₈cycloalkyl, C₃₋₈cycloalkylmethyl, phenyl or phenylC₁₋₂alkyl, wherein any C₃₋₈cycloalkyl or phenyl moiety is optionally substituted by 1, 2 or 3 substituents which may be the same or different, selected from R⁸, wherein R⁸ is halogen, C₁₋₄alkyl, C₁₋₄alkoxy and C₁₋₄haloalkyl. Alternatively, R⁷ represents C₁₋₆alkyl, C₂₋₆alkenyl, C₂₋₆alkynyl, C₁₋₆haloalkyl or C₁₋₄alkoxyC₁₋₆alkyl; or C₃₋₆cycloalkyl, C₃₋₆cycloalkylmethyl, phenyl or phenylC₁₋₂alkyl, wherein any C₃₋₆cycloalkyl or phenyl moiety is optionally substituted by 1, 2 or 3 substituents which may be the same or different, selected from R⁸, wherein R⁸ is halogen, C₁₋₄alkyl, C₁₋₄alkoxy and C₁₋₄haloalkyl.

Even more preferably, R⁷ represents C₁₋₆alkyl, C₃₋₈cycloalkyl, phenyl or phenylC₁₋₂alkyl, wherein phenyl or phenylC₁₋₂alkyl are optionally substituted by 1 or 2 R⁸ substituents independently selected from halogen, C₁₋₄alkyl, C₁₋₄haloalkyl and C₁₋₄alkoxy. Alternatively, R⁷ represents C₁₋₆alkyl, C₃₋₆cycloalkyl, phenyl or phenylC₁₋₂alkyl, wherein any C₃₋₆cycloalkyl or phenyl moiety is optionally substituted by 1 or 2 substituents, which may be the same or different, selected from R⁸, wherein R⁸ is halogen, C₁₋₄alkyl, C₁₋₄haloalkyl and C₁₋₄alkoxy.

Further more preferably, R⁷ represents C₁₋₄alkyl, C₁₋₃fluoroalkyl, C₅₋₆cycloalkyl, phenyl or phenylC₁₋₂alkyl, wherein C₅₋₆cycloalkyl, phenyl or phenylC₁₋₂alkyl are optionally substituted with 1 or 2 R⁸ substituents independently selected from fluorine, chlorine, methyl, ethyl, methoxy, ethoxy, difluoromethyl and trifluoromethyl. Alternatively, R⁷ represents C₁₋₄alkyl, C₁₋₃fluoroalkyl, C₅₋₆cycloalkyl, phenyl or phenylC₁₋₂alkyl, wherein any C₅₋₆cycloalkyl or phenyl moiety is optionally substituted by 1 or 2 substituents, which may be the same or different, selected from R⁸, wherein R⁸ is fluorine, chlorine, methyl, ethyl, methoxy, ethoxy, difluoromethyl and trifluoromethyl.

In certain embodiments of the invention, when R⁷ represents C₃₋₈cycloalkyl, C₃₋₈cycloalkylC₁₋₃alkyl, phenyl, phenylC₁₋₃alkyl, heteroaryl, heteroarylC₁₋₃alkyl, heterocyclyl or heterocyclylC₁₋₃alkyl, any cycloalkyl, phenyl, heteroaryl or heterocyclyl moiety may optionally be substituted by 1, 2, 3, 4 or 5 substituents, which may be the same or different, selected from R⁸. Likewise, when R⁷ represents C₃₋₈cycloalkyl, C₃₋₈cycloalkylC₁₋₂alkyl, phenyl, phenylC₁₋₂alkyl, heteroaryl, heteroarylC₁₋₂alkyl, heterocyclyl or heterocyclylC₁₋₂alkyl, any cycloalkyl, phenyl, heteroaryl or heterocyclyl moiety may optionally be substituted by 1, 2 or 3 substituents, which may be the same or different, selected from R⁸. That is, where any alkylene fragment connecting R⁷ to the rest of the molecule is not substituted by R⁸.

Where R⁷ is optionally substituted by 1, 2, 3, 4 or 5 substituents, which may the same or different, selected from R⁸, this includes where R⁷ may be optionally substituted by 1, 2, 3 or 4; 1, 2 or 3; or 1 or 2 substituents, which may the same or different, selected from R⁸, or where R⁷ may be optionally substituted by a single substituent, selected from R⁸.

According to the invention, where R⁷ represents heteroaryl or heterocyclyl, the R⁷ substituent is bonded to the rest of the compound according to Formula (I) (ie, to L¹) through a carbon atom. When R⁷ represents heteroarylC₁₋₃alkyl or heterocyclylC₁₋₃alkyl, the R⁷ substituent is bonded to the rest of the compound according to Formula (I) (ie, to L¹) through a carbon atom on the C₁₋₃alkyl moiety.

R⁸ represents cyano, halogen, hydroxy, C₁₋₄alkyl, C₁₋₄haloalkyl, C₁₋₄alkoxy or C₁₋₄haloalkoxy. Preferably, R⁸ is selected from halogen, C₁₋₄alkyl, C₁₋₄haloalkyl and C₁₋₄alkoxy. More preferably, R⁸ is fluorine, chlorine, methyl, ethyl, difluoromethyl, trifluoromethyl, methoxy and ethoxy.

Preferably, A¹ represents N or CR¹, wherein R¹ is selected from hydrogen or methyl;
R², R³ and R⁴ are hydrogen;
n is 0 or 1;
R⁵ and R⁶ are hydrogen, or R⁵ is hydrogen and R⁶ is methyl;
is S, S(O) or S(O)₂; and
R⁷ represents hydrogen, C₁₋₆alkyl, C₂₋₆alkenyl, C₂₋₆alkynyl, cyanoC₁₋₆alkyl, C₁₋₆haloalkyl or C₁₋₄alkoxyC₁₋₆alkyl; or C₃₋₈cycloalkyl or C₃₋₈cycloalkylC₁₋₂alkyl wherein the cycloalkyl moiety is optionally partially unsaturated, phenyl or phenylC₁₋₂alkyl, heteroaryl bonded to L¹ through a carbon atom or heteroarylC₁₋₂alkyl wherein the heteroaryl moiety is a 5- or 6-membered monocyclic aromatic ring which comprises 1, 2, 3 or 4 heteroatoms individually selected from N, O and S, or heterocyclyl bonded to L¹ through a carbon atom or heterocyclylC₁₋₂alkyl wherein the heterocyclyl moiety is a 4- to 6-membered non-aromatic ring which comprises 1, 2 or 3 heteroatoms individually selected from N, O and S; wherein any C₃₋₈cycloalkyl or C₃₋₈cycloalkylC₁₋₂alkyl, phenyl or phenylC₁₋₂alkyl, heteroaryl or heteroarylC₁₋₂alkyl, or heterocyclyl or heterocyclylC₁₋₂alkyl moiety is optionally substituted by 1, 2, or 3 substituents, which may be the same or different, selected from R⁸; wherein R⁸ represents halogen, C₁₋₄alkyl, C₁₋₄haloalkyl and C₁₋₄alkoxy.

More preferably, A¹ represents N or CR¹, wherein R¹ is selected from hydrogen or methyl;
R², R³ and R⁴ are hydrogen;
n is 0 or 1;
R⁵ and R⁶ are hydrogen, or R⁵ is hydrogen and R⁶ is methyl;
L¹ is S, S(O) or S(O)₂; and
R⁷ represents C₁₋₆alkyl, C₂₋₆alkenyl, C₂₋₆alkynyl, C₁₋₆haloalkyl or C₁₋₄alkoxyC₁₋₆alkyl; or C₃₋₈cycloalkyl, C₃₋₈cycloalkylmethyl, phenyl or phenylC₁₋₂alkyl, wherein any C₃₋₈cycloalkyl or phenyl moiety is optionally substituted by 1, 2 or 3 substituents which may be the same or different, selected from R⁸, wherein R⁸ is halogen, C₁₋₄alkyl, C₁₋₄alkoxy and C₁₋₄haloalkyl.

Even more preferably, A¹ represents N or CR¹, wherein R¹ is selected from hydrogen or methyl;
R², R³ and R⁴ are hydrogen;
n is 0;
is S or S(O); and
R⁷ represents C₁₋₆alkyl, C₃₋₈cycloalkyl, phenyl or phenylC₁₋₂alkyl, wherein phenyl or phenylC₁₋₂alkyl are optionally substituted with 1 or 2 R⁸ substituents independently selected from halogen, C₁₋₄alkyl, C₁₋₄haloalkyl and C₁₋₄alkoxy.

Most preferably, A¹ represents N or CR¹, wherein R¹ is hydrogen;
R², R³ and R⁴ are hydrogen;
n is 0;
L¹ is S or S(O); and
R⁷ represents C₁₋₄alkyl, C₁₋₃fluoroalkyl, C₅₋₆cycloalkyl, phenyl or phenylC₁₋₂alkyl, wherein C₅₋₆cycloalkyl, phenyl or phenylC₁₋₂alkyl are optionally substituted with 1 or 2 R⁸ substituents independently selected from fluorine, chlorine, methyl, ethyl, methoxy, ethoxy, difluoromethyl and trifluoromethyl.

Preferably, the compound according to Formula (I) is selected from a compound listed in Table T1, Table T2 or Table T3 below.

The compounds of Formula (I) may be enantiomers (when n=1) as represented by Formula (I-a) or Formula (I-b) below when R⁵ and R⁶ are different substituents.

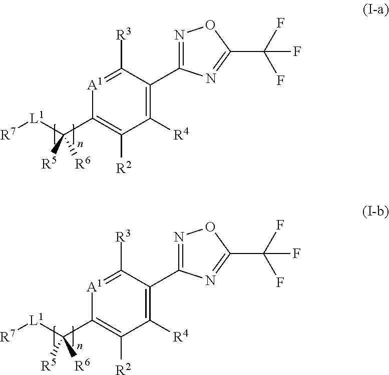

Likewise, the compounds of the present invention may be enantiomers (when n=2) when at one of the two carbon positions bound to R⁵ and R⁶, R⁵ and R⁶ are different substituents and at the other carbon position, R⁵ and R⁶ are the same. Alternatively, the compounds of Formula (I) may be diastereomers (when n=2) when at each of the two carbon positions bound to R⁵ and R⁶, R⁵ and R⁶ are different.

It is understood that when in aqueous media, the compounds of formula (I) according to the invention may be present in a reversible equilibrium with the corresponding covalently hydrated forms (ie, the compounds of formula (I-I) and formula (I-II) as shown below) at the CF₃-oxadiazole motif. This dynamic equilibrium may be important for the biological activity of the compounds of Formula (I). The designations of n, A¹, R¹, R², R³, R⁴, R⁵, R⁶, R⁷, and R⁸ with reference to the compounds of formula (I) of the present invention apply generally to the compounds of Formula (I-I) and Formula (I-II), as well as to the specific disclosures of combinations of n, A¹, R¹, R², R³, R⁴, R⁵, R⁶, R⁷, and R⁸ as represented in Tables 1A to 18A, 1B to 18B, and 1C to 18C below or the compounds 1.1 to 1.27 described in Table T1 (below), the compounds 2.1 to 2.32 described in Table T2 (below), and the compounds 3.1 to 3.24 described in Table T3 (below).

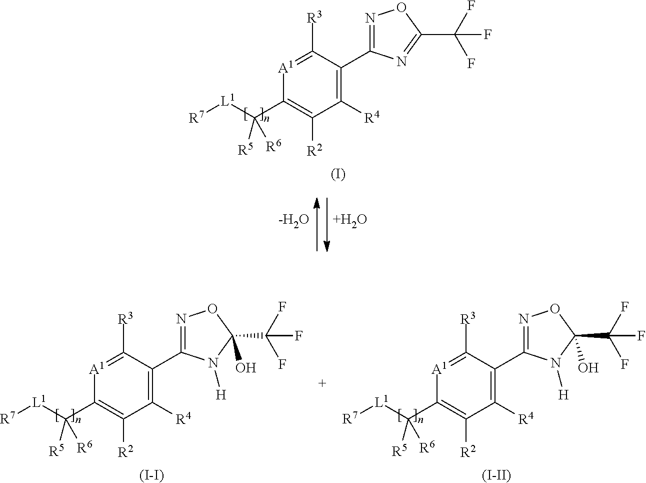

Compounds of the present invention can be made as shown in the following schemes 1 to 13, in which, unless otherwise stated, the definition of each variable is as defined above for a compound of formula (I).

Compounds of formula (I), wherein L¹ is S(O) or S(O)₂, can be prepared from compounds of formula (II) via treatment with oxidant (e.g. m-chloroperoxybenzoic acid or hydrogen peroxide) in a suitable solvent (e.g., chloroform, dichloromethane or glacial acetic acid) at a temperature between −10° C. and 25° C. For related examples, see Hendriks, C. M. M. et al Adv. Synth. Catal. (2013), 3363 and Russell, G. A. Pecoraro, J. M. J. Org. Chem. (1979), 44, 3990. This reaction is shown in Scheme 1.

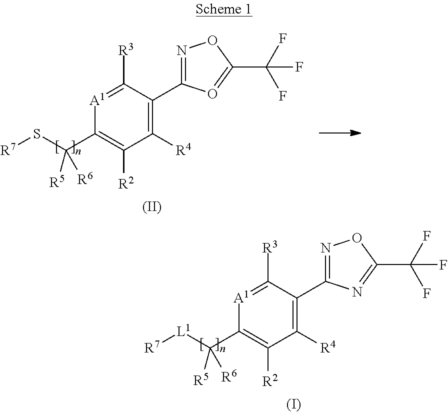

Compounds of formula (II) can be prepared from compounds of formula (IV), wherein X is Cl or Br, via treatment with thiols of formula (III), in the presence of a suitable base, (e.g., NaH or potassium carbonate), in a suitable solvent, (e.g., dimethylsulfoxide) at a temperature between 0° C. and 100° C. In some cases, a greater reaction performance may be gained with microwaves irradiation. For related examples, see Russell, G. A., Pecoraro, J. M. J. Org. Chem. (1979) 44, 3990 and Tsunoda, T. et al Tetrahedron Lett. (1999) 40, 7359. This is shown in reaction Scheme 2.

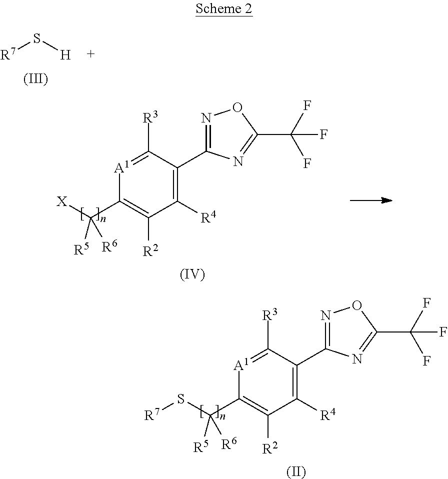

Alternatively, compounds of formula (II) can be prepared from compounds of formula (V), wherein X is Cl, Br, I or —OS(O)₂Me, via treatment with thiols of formula (VI), in the presence of a suitable base, (e.g., NaH or potassium carbonate), in a suitable solvent, (e.g., dimethylsulfoxide) at a temperature between 0° C. and 100° C. In some cases, a greater reaction performance may be gained with microwaves irradiation. For related examples, see Park, N. et al J. Org. Chem. (2011), 76, 4371; Verma, A. K. et al Tetrahedron Lett. (2007), 48, 7199 and WO 2002/000632. Compounds of formula (III) are commercially available or prepared using known methods. This reaction is shown in Scheme 3.

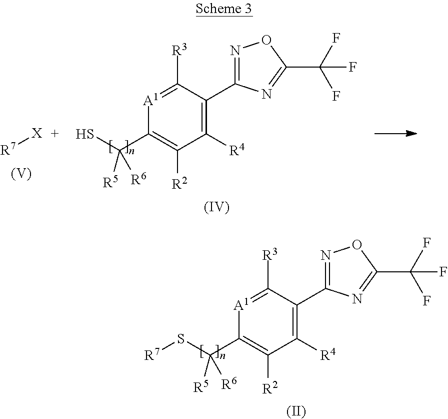

Compounds of formula (VI) (ie, compound of formula (IV) when n is 1) can be prepared from compounds of formula (VII), wherein X is Cl, or Br, via treatment with a sulphur source (e.g., thioacetic acid) and a suitable base (e.g., pyridine or potassium carbonate) in a suitable solvent, (e.g., tetrahydrofuran or acetone) at a temperature between 20° and 25° C. For related examples, see Han, C.-C., Balakumar, R. Tetrahedron Lett. (2006) 47, 8255 and WO 2010/087377. This reaction is shown in Scheme 4.

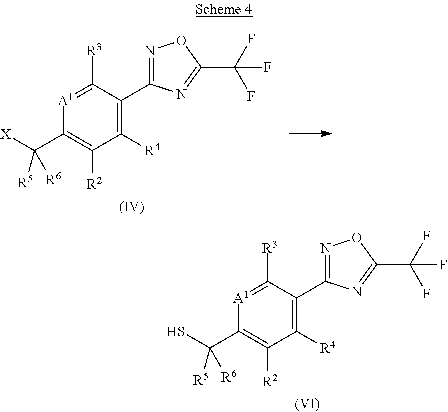

Compounds of formula (VII), wherein X is Cl or Br can be prepared from compounds of formula (VIII), by treatment with a halogen source (e.g., N-bromosuccimide (NBS) or N-chlorosuccimide (NCS)) and a radical initiator (eg., (PhCO₂)₂ or azobisisobutyronitrile (AIBN)) in a suitable solvent, such as tetrachloromethane, at temperatures between 55° and 100° C. in the presence of ultraviolet light. For related examples, see Liu, S. et al. Synthesis (2001), 14, 2078 and Kompella, A. et al. Org. Proc. Res. Dev. (2012), 16, 1794. This reaction is shown in Scheme 5.

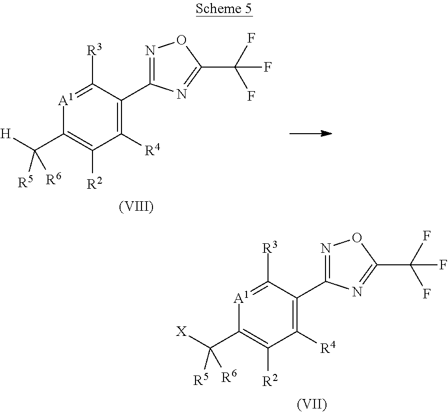

Additionally, compounds of formula (II) can be prepared from compounds of formula (IX) by treatment with trifluoroacetic anhydride in the presence of a base (e.g., pyridine or 4-dimethylaminopyridine) in a suitable solvent (e.g., tetrahydrofuran or ethanol) at a temperature between 25° C. and 75° C. For related examples, see WO 2003/028729 and WO 2010/045251. This reaction is shown in Scheme 6.

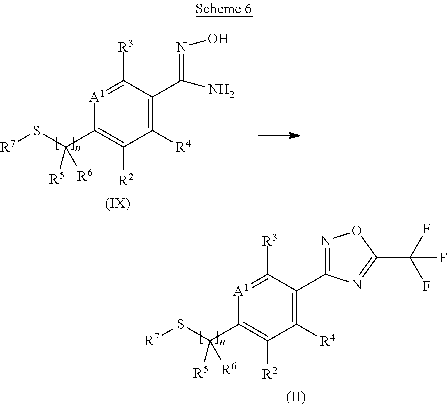

Compounds of formula (IX) can be prepared from compounds of formula (X) by treating them with a hydroxylamine hydrochloride salt in the presence of a base, such as triethylamine, in a suitable solvent, such as methanol, at a temperature between 0° C. and 100° C. For related examples, see Kitamura, S. et al. Chem. Pharm. Bull. (2001), 49, 268 and WO 2013/066838. This reaction is shown in Scheme 7.

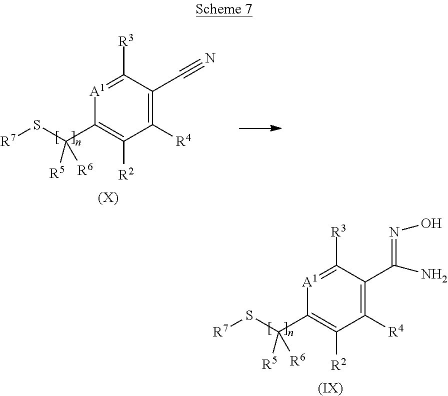

Compounds of formula (X) can be prepared from compounds of formula (XI), wherein Y is Br or I, via a metal-promoted reaction with a suitable cyanide reagent, such as Pd(O)/Zn(CN)₂ or CuCN, in a suitable solvent (e.g., dimethylformamide or N-methylpyrrolidone) at elevated temperature between 100° C. and 120° C. For related examples, see Rutan, K. J. et al J. Org. Chem., (1995), 60, 2948; WO 2013/130935; and De Benedetti, P. G. et al J. Chem. Soc., Perk. Trans 2 (1985), 10, 1527. This reaction is shown in Scheme 8.

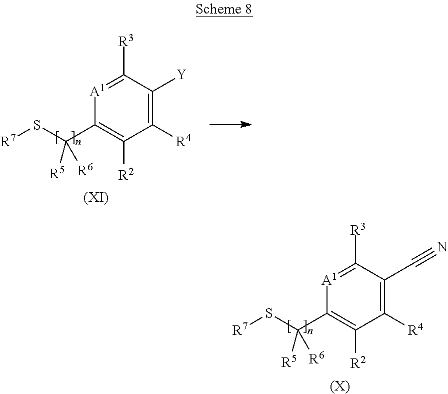

Compounds of formula (XII), wherein Z is Br, I or CN, can be prepared from compounds of formula (XIII), wherein X is Cl, Br, I or OS(O)₂Me by treatment with thiols of formula (III), in the presence of a base (e.g., NaH or sodium carbonate) in a suitable solvent (e.g., dimethyl sulfoxide, dichloromethane or ethanol) at a temperature between 0° C. and 100° C. In some cases, a better reaction performance may be gained from use of a catalyst (e.g., CuI) and microwaves irradiation. For related examples, see Wang, B. et al Organic Chemistry Frontiers (2015), 2, 973; WO 2012/9931088; Uyeda, C. et al J. Am. Chem. Soc. (2013), 135, 9548; and Santoni, G. et al Chem. Eur. J. (2010), 16, 645. Compounds of formula (III) are commercially available. This reaction is shown in Scheme 9.

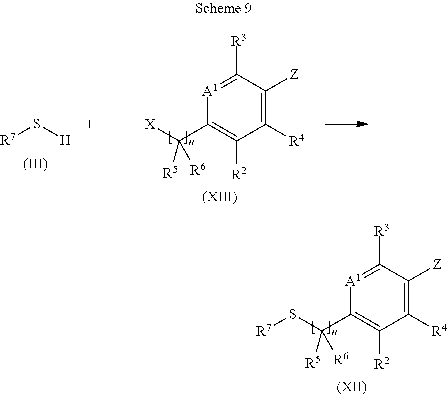

Alternatively, compounds of formula (XII), wherein Z is Br, I or CN, can be prepared from compounds of formula (V), wherein X is Cl, Br, I or OS(O)₂Me, via treatment with thiols of formula (XIV), in the presence of a suitable base, (e.g., NaH or potassium carbonate), in a suitable solvent (e.g., dimethylsulfoxide or ethanol) at a temperature between 0° C. and 100° C. In some cases, a greater reaction performance may be gained with microwaves irradiation. For related examples, see Park, N. et al J. Org. Chem., (2011), 76, 4371; Verma, A. K. et al Tetrahedron Lett. (2007), 48, 7199; and WO 2002/000632. Compounds of formula (V) are commercially available. This reaction is shown in Scheme 10.

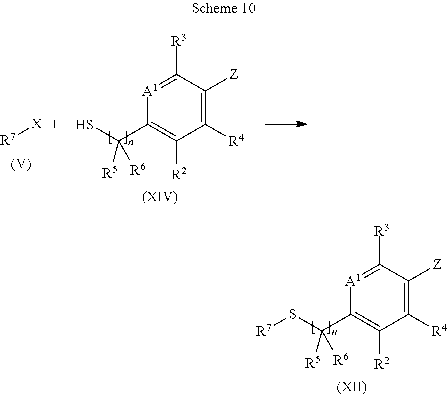

Compounds of formula (XIV), wherein Z is Br, I or CN, are either commercially available or are prepared from compounds of formula (XIII), wherein X is Cl or Br, by treatment with a sulphur source (e.g., thioacetic acid) and a suitable base (e.g., pyridine or potassium carbonate) in a suitable solvent (e.g., tetrahydrofuran or acetone) at a temperature between 0° and 25° C. For related examples, see Han, C.-C., Balakumar, R. Tetrahedron Lett. (2006) 47, 8255 and WO 2010/087377. This reaction is shown in Scheme 11.

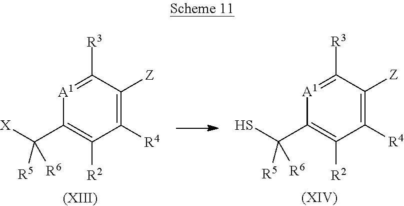

Compounds of formula (XIII), wherein X is Cl or Br, are either commercially available or can be prepared from compounds of formula (XV), wherein Z is Br, I, or CN, by treatment with a halogen source, (e.g., N-bromosuccimide (NBS) or N-chlorosuccimide (NCS)) and a radical initiator, such as (PhCO₂)₂ or azobisisobutyronitrile (AIBN), in the presence of ultraviolet light, in a suitable solvent, such as tetrachloromethane, at temperatures between 55° C. and 100° C. For related examples, see Liu, S. et al. Syntheis (2001), 14, 2078 and Kompella, A. et al. Org. Proc. Res. Dev. (2012), 16, 1794. Compounds of formula (XV) are commercially available. This reaction is shown in Scheme 12.

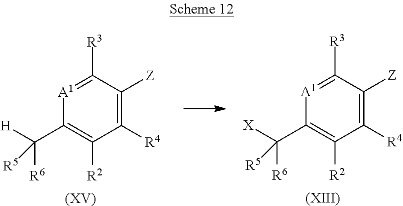

Alternatively, compounds of formula (XIII), wherein X is Cl, Br, I, or OS(O)₂Me and Z is Br, I, or CN are either commercially available or can be prepared from compounds of formula (XVI), by treatment with a halogen source (eg, CBr₄, CCl₄ or I₂) in the presence of triphenylphosphine, or with methanesulfonyl chloride (CIS(O)₂Me), in a suitable solvent, (eg, dichloromethane or 1,2-dichloroethane) at a temperature between 0° C. and 40° C. For related examples, see Liu, H. et al Bioorg. Med. Chem. (2008), 16, 10013; WO 2014/020350; and Kompella, A. et al Bioorg. Med. Chem. Lett. (2001), 1, 3161. Compounds of formula (XVI) are commercially available. This reaction is shown in Scheme 13.

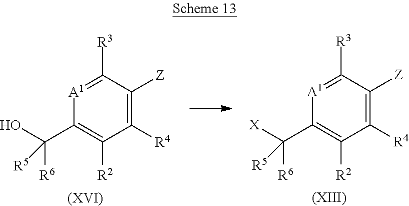

As already indicated, surprisingly, it has now been found that the novel compounds of formula (I) according to the invention have, for practical purposes, a very advantageous level of biological activity for protecting plants against diseases that are caused by fungi.

The compounds of formula (I) can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of formula I can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) is applied to the plants, to parts thereof or the locus thereof.

It is also possible to use compounds of formula (I) as fungicide. The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of formula (I) as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of formula (I) before planting: seed, for example, can be dressed before being sown. The active compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of formula (I) can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula (I) are for example, effective against fungi and fungal vectors of disease as well as phytopathogenic bacteria and viruses. These fungi and fungal vectors of disease as well as phytopathogenic bacteria and viruses are for example:

Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Diaporthe spp, Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp. including T. harzianum, T. pseudokoningii, T. viride, Trichophyton spp, Typhula spp, Uncinula necator, Urocystis spp, Ustilago spp, Venturia spp. including V. inaequalis, Verticillium spp, and Xanthomonas spp.

The compounds of formula (I) may be used for example on turf, ornamentals, such as flowers, shrubs, broad-leaved trees or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO93/07278, WO95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of formula I may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimeter to 1 centimeter and preferably 1 to 2 millimeters in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of formula (I) are normally used in the form of agrochemical compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) may be used in the form of (fungicidal) compositions for controlling or protecting against phytopathogenic microorganisms, comprising as active ingredient at least one compound of formula (I) or of at least one preferred individual compound as defined herein, in free form or in agrochemically usable salt form, and at least one of the above-mentioned adjuvants.

The invention therefore provides a composition, preferably a fungicidal composition, comprising at least one compound formula (I) an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally-active compounds, for example an additional fungicidal active ingredient in addition to the compound of formula (I).

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following: acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients also include the following: 3-difluoromethyl-1-methyl-1H-pyrazole-4-carboxylic acid (9-dichloromethylene-1,2,3,4-tetrahydro-1,4-methano-naphthalen-5-yl)-amide, 3-difluoromethyl-1-methyl-1H-pyrazole-4-carboxylic acid methoxy-[1-methyl-2-(2,4,6-trichlorophenyl)-ethyl]-amide, 1-methyl-3-difluoromethyl-1H-pyrazole-4-carboxylic acid (2-dichloromethylene-3-ethyl-1-methyl-indan-4-yl)-amide (1072957-71-1), 1-methyl-3-difluoromethyl-1H-pyrazole-4-carboxylic acid (4′-methylsulfanyl-biphenyl-2-yl)-amide, 1-methyl-3-difluoromethyl-4H-pyrazole-4-carboxylic acid [2-(2,4-dichloro-phenyl)-2-methoxy-1-methyl-ethyl]amide, (5-Chloro-2,4-dimethyl-pyridin-3-yl)-(2,3,4-trimethoxy-6-methyl-phenyl)-methanone, (5-Bromo-4-chloro-2-methoxy-pyridin-3-yl)-(2,3,4-trimethoxy-6-methyl-phenyl)-methanone, 2-{2-[(E)-3-(2,6-Dichloro-phenyl)-1-methyl-prop-2-en-(E)-ylideneaminooxymethyl]-phenyl}-2-[(Z)-methoxyimino]-N-methyl-acetamide, 3-[5-(4-Chloro-phenyl)-2,3-dimethyl-isoxazolidin-3-yl]-pyridine, (E)-N-methyl-2-[2-(2, 5-dimethylphenoxymethyl) phenyl]-2-methoxy-iminoacetamide, 4-bromo-2-cyano-N, N-dimethyl-6-trifluoromethylbenzimidazole-1-sulphonamide, a-[N-(3-chloro-2, 6-xylyl)-2-methoxyacetamido]-y-butyrolactone, 4-chloro-2-cyano-N,-dimethyl-5-p-tolylimidazole-1-sulfonamide, N-allyl-4, 5,-dimethyl-2-trimethylsilylthiophene-3-carboxamide, N-(I-cyano-1, 2-dimethylpropyl)-2-(2, 4-dichlorophenoxy) propionamide, N-(2-methoxy-5-pyridyl)-cyclopropane carboxamide, (.+−.)-cis-1-(4-chlorophenyl)-2-(1H-1,2,4-triazol-1-yl)-cycloheptanol, 2-(1-tert-butyl)-1-(2-chlorophenyl)-3-(1,2,4-triazol-1-yl)-propan-2-ol, 2′,6′-dibromo-2-methyl-4-trifluoromethoxy-4′-trifluoromethyl-1,3-thiazole-5-carboxanilide, 1-imidazolyl-1-(4′-chlorophenoxy)-3,3-dimethylbutan-2-one, methyl (E)-2-[2-[6-(2-cyanophenoxy)pyrimidin-4-yloxy]phenyl]3-methoxyacrylate, methyl (E)-2-[2-[6-(2-thioamidophenoxy)pyrimidin-4-yloxy]phenyl]-3-methoxyacrylate, methyl (E)-2-[2-[6-(2-fluorophenoxy)pyrimidin-4-yloxy]phenyl]-3-methoxyacrylate, methyl (E)-2-[2-[6-(2,6-difluorophenoxy)pyrimidin-4-yloxy]phenyl]-3-methoxyacrylate, methyl (E)-2-[2-[3-(pyrimidin-2-yloxy)phenoxy]phenyl]-3-methoxyacrylate, methyl (E)-2-[2-[3-(5-methyl pyrimidin-2-yloxy)-phenoxy]phenyl]-3-methoxyacrylate, methyl (E)-2-[2-[3-(phenyl-sulphonyloxy)phenoxy]phenyl-3-methoxyacrylate, methyl (E)-2-[2-[3-(4-nitrophenoxy)phenoxy]phenyl]-3-methoxyacrylate, methyl (E)-2-[2-phenoxyphenyl]-3-methoxyacrylate, methyl (E)-2-[2-(3,5-dimethyl-benzoyl)pyrrol-1-yl]-3-methoxyacrylate, methyl (E)-2-[2-(3-methoxyphenoxy)phenyl]-3-methoxyacrylate, methyl (E)-2[2-(2-phenylethen-1-yl)-phenyl]-3-methoxyacrylate, methyl (E)-2-[2-(3,5-dichlorophenoxy)pyridin-3-yl]-3-methoxyacrylate, methyl (E)-2-(2-(3-(1,1,2,2-tetrafluoroethoxy)phenoxy)phenyl)-3-methoxyacrylate, methyl (E)-2-(2-[3-(alpha-hydroxybenzyl)phenoxy]phenyl)-3-methoxyacrylate, methyl (E)-2-(2-(4-phenoxypyridin-2-yloxy)phenyl)-3-methoxyacrylate, methyl (E)-2-[2-(3-n-propyloxy-phenoxy)phenyl]3-methoxyacrylate, methyl (E)-2-[2-(3-isopropyloxyphenoxy)phenyl]-3-methoxyacrylate, methyl (E)-2-[2-[3-(2-fluorophenoxy)phenoxy]phenyl]-3-methoxyacrylate, methyl (E)-2-[2-(3-ethoxyphenoxy)phenyl]-3-methoxyacrylate, methyl (E)-2-[2-(4-tert-butyl-pyridin-2-yloxy)phenyl]-3-methoxyacrylate, methyl (E)-2-[2-[3-(3-cyanophenoxy)phenoxy]phenyl]-3-methoxyacrylate, methyl (E)-2-[2-[(3-methyl-pyridin-2-yloxymethyl)phenyl]-3-methoxyacrylate, methyl (E)-2-[2-[6-(2-methyl-phenoxy)pyrimidin-4-yloxy]phenyl]-3-methoxyacrylate, methyl (E)-2-[2-(5-bromo-pyridin-2-yloxymethyl)phenyl]-3-methoxyacrylate, methyl (E)-2-[2-(3-(3-iodopyridin-2-yloxy)phenoxy)phenyl]-3-methoxyacrylate, methyl (E)-2-[2-[6-(2-chloropyridin-3-yloxy)pyrimidin-4-yloxy]phenyl]-3-methoxyacrylate, methyl (E),(E)-2-[2-(5,6-dimethylpyrazin-2-ylmethyloximinomethyl)phenyl]-3-methoxyacrylate, methyl (E)-2-{2-[6-(6-methylpyridin-2-yloxy)pyrimidin-4-yloxy]phenyl}-3-methoxy-acrylate, methyl (E),(E)-2-{2-(3-methoxyphenyl)methyloximinomethyl]phenyl}-3-methoxyacrylate, methyl (E)-2-{2-(6-(2-azidophenoxy)-pyrimidin-4-yloxy]phenyl}-3-methoxyacrylate, methyl (E),(E)-2-{2-[6-phenylpyrimidin-4-yl)-methyloximinomethyl]phenyl}-3-methoxyacrylate, methyl (E),(E)-2-{2-[(4-chlorophenyl)-methyloximinomethyl]-phenyl}-3-methoxyacrylate, methyl (E)-2-{2-[6-(2-n-propylphenoxy)-1,3,5-triazin-4-yloxy]phenyl}-3-methoxyacrylate, methyl (E),(E)-2-{2-[(3-nitrophenyl)methyloximinomethyl]phenyl}-3-methoxyacrylate, 3-chloro-7-(2-aza-2,7,7-trimethyl-oct-3-2,6-dichloro-N-(4-trifluoromethylbenzyl)-benzamide, 3-iodo-2-propinyl alcohol, 4-chlorophenyl-3-iodopropargyl formal, 3-bromo-2,3-diiodo-2-propenyl ethylcarbamate, 2,3,3-triiodoallyl alcohol, 3-bromo-2,3-diiodo-2-propenyl alcohol, 3-iodo-2-propinyl n-butylcarbamate, 3-iodo-2-propinyl n-hexylcarbamate, 3-iodo-2-propinyl cyclohexyl-carbamate, 3-iodo-2-propinyl phenylcarbamate; phenol derivatives, such as tribromophenol, tetrachlorophenol, 3-methyl-4-chlorophenol, 3,5-dimethyl-4-chlorophenol, phenoxyethanol, dichlorophene, o-phenylphenol, m-phenylphenol, p-phenylphenol, 2-benzyl-4-chlorophenol, 5-hydroxy-2(5H)-furanone; 4,5-dichlorodithiazolinone, 4,5-benzodithiazolinone, 4,5-trimethylenedithiazolinone, 4,5-dichloro-(3H)-1,2-dithiol-3-one, 3,5-dimethyl-tetrahydro-1,3,5-thiadiazine-2-thione, N-(2-p-chlorobenzoylethyl)-hexaminium chloride, acibenzolar, acypetacs, alanycarb, albendazole, aldimorph, allicin, allyl alcohol, ametoctradin, amisulbrom, amobam, ampropylfos, anilazine, asomate, aureofungin, azaconazole, azafendin, azithiram, azoxystrobin, barium polysulfide, benalaxyl, benalaxyl-M, benodanil, benomyl, benquinox, bentaluron, benthiavalicarb, benthiazole, benzalkonium chloride, benzamacril, benzamorf, benzohydroxamic acid, benzovindiflupyr, berberine, bethoxazin, biloxazol, binapacryl, biphenyl, bitertanol, bithionol, bixafen, blasticidin-S, boscalid, bromothalonil, bromuconazole, bupirimate, buthiobate, butylamine calcium polysulfide, captafol, captan, carbamorph, carbendazim, carbendazim chlorhydrate, carboxin, carpropamid, carvone, CGA41396, CGA41397, chinomethionate, chitosan, chlobenthiazone, chloraniformethan, chloranil, chlorfenazole, chloroneb, chloropicrin, chlorothalonil, chlorozolinate, chlozolinate, climbazole, clotrimazole, clozylacon, copper containing compounds such as copper acetate, copper carbonate, copper hydroxide, copper naphthenate, copper oleate, copper oxychloride, copper oxyquinolate, copper silicate, copper sulphate, copper tallate, copper zinc chromate and Bordeaux mixture, cresol, cufraneb, cuprobam, cuprous oxide, cyazofamid, cyclafuramid, cycloheximide, cyflufenamid, cymoxanil, cypendazole, cyproconazole, cyprodinil, dazomet, debacarb, decafentin, dehydroacetic acid, di-2-pyridyl disulphide 1, 1′-dioxide, dichlofluanid, diclomezine, dichlone, dicloran, dichlorophen, dichlozoline, diclobutrazol, diclocymet, diethofencarb, difenoconazole, difenzoquat, diflumetorim, 0, 0-di-iso-propyl-S-benzyl thiophosphate, dimefluazole, dimetachlone, dimetconazole, dimethomorph, dimethirimol, diniconazole, diniconazole-M, dinobuton, dinocap, dinocton, dinopenton, dinosulfon, dinoterbon, diphenylamine, dipyrithione, disulfiram, ditalimfos, dithianon, dithioether, dodecyl dimethyl ammonium chloride, dodemorph, dodicin, dodine, doguadine, drazoxolon, edifenphos, enestroburin, epoxiconazole, etaconazole, etem, ethaboxam, ethirimol, ethoxyquin, ethilicin, ethyl (Z)—N-benzyl-N([methyl (methyl-thioethylideneamino-oxycarbonyl) amino] thio)-ß-alaninate, etridiazole, famoxadone, fenamidone, fenaminosulf, fenapanil, fenarimol, fenbuconazole, fenfuram, fenhexamid, fenitropan, fenoxanil, fenpiclonil, fenpropidin, fenpropimorph, fenpyrazamine, fentin acetate, fentin hydroxide, ferbam, ferimzone, fluazinam, fludioxonil, flumetover, flumorph, flupicolide, fluopyram, fluoroimide, fluotrimazole, fluoxastrobin, fluquinconazole, flusilazole, flusulfamide, flutanil, flutolanil, flutriafol, fluxapyroxad, folpet, formaldehyde, fosetyl, fuberidazole, furalaxyl, furametpyr, furcarbanil, furconazole, furfural, furmecyclox, furophanate, glyodin, griseofulvin, guazatine, halacrinate, hexa chlorobenzene, hexachlorobutadiene, hexachlorophene, hexaconazole, hexylthiofos, hydrargaphen, hydroxyisoxazole, hymexazole, imazalil, imazalil sulphate, imibenconazole, iminoctadine, iminoctadine triacetate, inezin, iodocarb, ipconazole, ipfentrifluconazole, iprobenfos, iprodione, iprovalicarb, isopropanyl butyl carbamate, isoprothiolane, isopyrazam, isotianil, isovaledione, izopamfos, kasugamycin, kresoxim-methyl, LY186054, LY211795, LY248908, mancozeb, mandipropamid, maneb, mebenil, mecarbinzid, mefenoxam, mefentrifluconazole, mepanipyrim, mepronil, mercuric chloride, mercurous chloride, meptyldinocap, metalaxyl, metalaxyl-M, metam, metazoxolon, metconazole, methasulfocarb, methfuroxam, methyl bromide, methyl iodide, methyl isothiocyanate, metiram, metiram-zinc, metominostrobin, metrafenone, metsulfovax, milneb, moroxydine, myclobutanil, myclozolin, nabam, natamycin, neoasozin, nickel dimethyldithiocarbamate, nitrostyrene, nitrothal-iso-propyl, nuarimol, octhilinone, ofurace, organomercury compounds, orysastrobin, osthol, oxadixyl, oxasulfuron, oxathiapiprolin, oxine-copper, oxolinic acid, oxpoconazole, oxycarboxin, parinol, pefurazoate, penconazole, pencycuron, penflufen, pentachlorophenol, penthiopyrad, phenamacril, phenazin oxide, phosdiphen, phosetyl-Al, phosphorus acids, phthalide, picoxystrobin, piperalin, polycarbamate, polyoxin D, polyoxrim, polyram, probenazole, prochloraz, procymidone, propamidine, propamocarb, propiconazole, propineb, propionic acid, proquinazid, prothiocarb, prothioconazole, pydiflumetofen, pyracarbolid, pyraclostrobin, pyrametrostrobin, pyraoxystrobin, pyrazophos, pyribencarb, pyridinitril, pyrifenox, pyrimethanil, pyriofenone, pyroquilon, pyroxychlor, pyroxyfur, pyrrolnitrin, quaternary ammonium compounds, quinacetol, quinazamid, quinconazole, quinomethionate, quinoxyfen, quintozene, rabenzazole, santonin, sedaxane, silthiofam, simeconazole, sipconazole, sodium pentachlorophenate, spiroxamine, streptomycin, sulphur, sultropen, tebuconazole, tebfloquin, tecloftalam, tecnazene, tecoram, tetraconazole, thiabendazole, thiadifluor, thicyofen, thifluzamide, 2-(thiocyanomethylthio) benzothiazole, thiophanate-methyl, thioquinox, thiram, tiadinil, timibenconazole, tioxymid, tolclofos-methyl, tolylfluanid, triadimefon, triadimenol, triamiphos, triarimol, triazbutil, triazoxide, tricyclazole, tridemorph, trifloxystrobin, triflumazole, triforine, triflumizole, triticonazole, uniconazole, urbacide, validamycin, valifenalate, vapam, vinclozolin, zarilamid, zineb, ziram, and zoxamide.

The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. No. 5,478,855, U.S. Pat. No. 4,639,771 and DE-19520936.

The compounds of the invention may be used in combination with derivatives and analogues of the general class of dioxomorpholine antiparasitic agents as described in WO 96/15121 and also with anthelmintic active cyclic depsipeptides such as those described in WO 96/11945, WO 93/19053, WO 93/25543, EP 0 626 375, EP 0 382 173, WO 94/19334, EP 0 382 173, and EP 0 503 538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in International Patent Application Publication Numbers WO 95/19363 or WO 04/72086, particularly the compounds disclosed therein.

Other examples of such biologically active compounds that the compounds of the invention may be used in combination with include but are not restricted to the following:

Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195, RH-0345, RH-2485, RYI-210, S-1283, S-1833, S1-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The following mixtures of the compounds of formula (I) with active ingredients are preferred. The abbreviation “TX” means one compound selected from the group consisting of the compounds described in Tables 1A to 18A, 1B to 18B, 1C to 18C (below), or Tables T1, T2 or T3 (below).

an adjuvant selected from the group of substances consisting of petroleum oils (628)+TX,

an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel [CCN]+TX, coumaphos (174)+TX, crotamiton [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos+TX, dicofol (242)+TX, dicrotophos (243)+TX, dienochlor (1071)+TX, dimefox (1081)+TX, dimethoate (262)+TX, dinactin (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram [CCN]+TX, disulfoton (278)+TX, DNOC (282)+TX, dofenapyn (1113)+TX, doramectin [CCN]+TX, endosulfan (294)+TX, endothion (1121)+TX, EPN (297)+TX, eprinomectin [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (473)+TX, isopropyl 0-(methoxyaminothiophosphoryl)salicylate (IUPAC name) (473)+TX, ivermectin [CCN]+TX, jasmolin I (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime [CCN]+TX, mipafox (1293)+TX, monocrotophos (561)+TX, morphothion (1300)+TX, moxidectin [CCN]+TX, naled (567)+TX, NC-184 (compound code)+TX, NC-512 (compound code)+TX, nifluridide (1309)+TX, nikkomycins [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos+TX, selamectin [CCN]+TX, SI-0009 (compound code)+TX, sophamide (1402)+TX, spirodiclofen (738)+TX, spiromesifen (739)+TX, SSI-121 (development code) (1404)+TX, sulfiram [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam+TX, tetrachlorvinphos (777)+TX, tetradifon (786)+TX, tetranactin (653)+TX, tetrasul (1425)+TX, thiafenox+TX, thiocarboxime (1431)+TX, thiofanox (800)+TX, thiometon (801)+TX, thioquinox (1436)+TX, thuringiensin [CCN]+TX, triamiphos (1441)+TX, triarathene (1443)+TX, triazophos (820)+TX, triazuron+TX, trichlorfon (824)+TX, trifenofos (1455)+TX, trinactin (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,

an anthelmintic selected from the group of substances consisting of abamectin (1)+TX, crufomate (1011)+TX, doramectin [CCN]+TX, emamectin (291)+TX, emamectin benzoate (291)+TX, eprinomectin [CCN]+TX, ivermectin [CCN]+TX, milbemycin oxime [CCN]+TX, moxidectin [CCN]+TX, piperazine [CCN]+TX, selamectin [CCN]+TX, spinosad (737) and thiophanate (1435)+TX,

an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal [CCN]+TX,

a biological agent selected from the group of substances consisting of Adoxophyes orana GV (12)+TX, Agrobacterium radiobacter (13)+TX, Amblyseius spp. (19)+TX, Anagrapha falcifera NPV (28)+TX, Anagrus atomus (29)+TX, Aphelinus abdominalis (33)+TX, Aphidius colemani (34)+TX, Aphidoletes aphidimyza (35)+TX, Autographa californica NPV (38)+TX, Bacillus firmus (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (53)+TX, Beauveria brongniartii (54)+TX, Chrysoperla carnea (151)+TX, Cryptolaemus montrouzieri (178)+TX, Cydia pomonella GV (191)+TX, Dacnusa sibirica (212)+TX, Diglyphus isaea (254)+TX, Encarsia formosa (scientific name) (293)+TX, Eretmocerus eremicus (300)+TX, Helicoverpa zea NPV (431)+TX, Heterorhabditis bacteriophora and H. megidis (433)+TX, Hippodamia convergens (442)+TX, Leptomastix dactylopii (488)+TX, Macrolophus caliginosus (491)+TX, Mamestra brassicae NPV (494)+TX, Metaphycus helvolus (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (575)+TX, Orius spp. (596)+TX, Paecilomyces fumosoroseus (613)+TX, Phytoseiulus persimilis (644)+TX, Spodoptera exigua multicapsid nuclear polyhedrosis virus (scientific name) (741)+TX, Steinernema bibionis (742)+TX, Steinernema carpocapsae (742)+TX, Steinernema feltiae (742)+TX, Steinernema glaseri (742)+TX, Steinernema riobrave (742)+TX, Steinernema riobravis (742)+TX, Steinernema scapterisci (742)+TX, Steinernema spp. (742)+TX, Trichogramma spp. (826)+TX, Typhlodromus occidentalis (844) and Verticillium lecanii (848)+TX,

a soil sterilant selected from the group of substances consisting of iodomethane (IUPAC name) (542) and methyl bromide (537)+TX,

a chemosterilant selected from the group of substances consisting of apholate [CCN]+TX, bisazir [CCN]+TX, busulfan [CCN]+TX, diflubenzuron (250)+TX, dimatif [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron [CCN]+TX, tepa [CCN]+TX, thiohempa [CCN]+TX, thiotepa [CCN]+TX, tretamine [CCN] and uredepa [CCN]+TX,

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin [CCN]+TX, brevicomin [CCN]+TX, codlelure [CCN]+TX, codlemone (167)+TX, cuelure (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure [CCN]+TX, ethyl 4-methyloctanoate (IUPAC name) (317)+TX, eugenol [CCN]+TX, frontalin [CCN]+TX, gossyplure (420)+TX, grandlure (421)+TX, grandlure I (421)+TX, grandlure II (421)+TX, grandlure III (421)+TX, grandlure IV (421)+TX, hexalure [CCN]+TX, ipsdienol [CCN]+TX, ipsenol [CCN]+TX, japonilure (481)+TX, lineatin [CCN]+TX, litlure [CCN]+TX, looplure [CCN]+TX, medlure [CCN]+TX, megatomoic acid [CCN]+TX, methyl eugenol (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure [CCN]+TX, oryctalure (317)+TX, ostramone [CCN]+TX, siglure [CCN]+TX, sordid in (736)+TX, sulcatol [CCN]+TX, tetradec-11-en-1-yl acetate (IUPAC name) (785)+TX, trimedlure (839)+TX, trimedlure A (839)+TX, trimedlure B₁ (839)+TX, trimedlure B₂ (839)+TX, trimedlure C (839) and trunc-call [CCN]+TX,

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,

an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin [CCN]+TX, allyxycarb (866)+TX, alpha-cypermethrin (202)+TX, alpha-ecdysone [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (41)+TX, azamethiphos (42)+TX, azinphos-ethyl (44)+TX, azinphos-methyl (45)+TX, azothoate (889)+TX, Bacillus thuringiensis delta endotoxins (52)+TX, barium hexafluorosilicate [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate+TX, bromfenvinfos (914)+TX, bromocyclen (918)+TX, bromo-DDT [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin+TX, cismethrin (80)+TX, clocythrin+TX, cloethocarb (999)+TX, closantel [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton [CCN]+TX, crotoxyphos (1010)+TX, crufomate (1011)+TX, cryolite (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate [CCN]+TX, d-limonene [CCN]+TX, d-tetramethrin (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos+TX, dicresyl [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin [CCN]+TX, DSP (1115)+TX, ecdysterone [CCN]+TX, El 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin [CCN]+TX, esfenvalerate (302)+TX, etaphos [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (473)+TX, isodrin (1235)+TX, isofenphos (1236)+TX, isolane (1237)+TX, isoprocarb (472)+TX, isopropyl O-(methoxyaminothiophosphoryl)salicylate (IUPAC name) (473)+TX, isoprothiolane (474)+TX, isothioate (1244)+TX, isoxathion (480)+TX, ivermectin [CCN]+TX, jasmolin I (696)+TX, jasmolin II (696)+TX, jodfenphos (1248)+TX, juvenile hormone I [CCN]+TX, juvenile hormone II [CCN]+TX, juvenile hormone III [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (533)+TX, methoxychlor (534)+TX, methoxyfenozide (535)+TX, methyl bromide (537)+TX, methyl isothiocyanate (543)+TX, methylchloroform [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime [CCN]+TX, mipafox (1293)+TX, mirex (1294)+TX, monocrotophos (561)+TX, morphothion (1300)+TX, moxidectin [CCN]+TX, naftalofos [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron [CCN]+TX, pentachlorophenol (623)+TX, pentachlorophenyl laurate (IUPAC name) (623)+TX, permethrin (626)+TX, petroleum oils (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I [CCN]+TX, precocene II [CCN]+TX, precocene Ill [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia [CCN]+TX, quinalphos (711)+TX, quinalphos-methyl (1376)+TX, quinothion (1380)+TX, quintiofos (1381)+TX, R-1492 (development code) (1382)+TX, rafoxanide [CCN]+TX, resmethrin (719)+TX, rotenone (722)+TX, RU 15525 (development code) (723)+TX, RU 25475 (development code) (1386)+TX, ryania (1387)+TX, ryanodine (traditional name) (1387)+TX, sabadilla (725)+TX, schradan (1389)+TX, sebufos+TX, selamectin [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron+TX, trichlorfon (824)+TX, trichlormetaphos-3 [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (725)+TX, veratrine (725)+TX, XMC (853)+TX, xylylcarb (854)+TX, YI-5302 (compound code)+TX, zeta-cypermethrin (205)+TX, zetamethrin+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO2005/077934)+TX,

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX, a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (210)+TX, dazomet (216)+TX, DBCP (1045)+TX, DCIP (218)+TX, diamidafos (1044)+TX, dichlofenthion (1051)+TX, dicliphos+TX, dimethoate (262)+TX, doramectin [CCN]+TX, emamectin (291)+TX, emamectin benzoate (291)+TX, eprinomectin [CCN]+TX, ethoprophos (312)+TX, ethylene dibromide (316)+TX, fenamiphos (326)+TX, fenpyrad+TX, fensulfothion (1158)+TX, fosthiazate (408)+TX, fosthietan (1196)+TX, furfural [CCN]+TX, GY-81 (development code) (423)+TX, heterophos [CCN]+TX, iodomethane (IUPAC name) (542)+TX, isamidofos (1230)+TX, isazofos (1231)+TX, ivermectin [CCN]+TX, kinetin (210)+TX, mecarphon (1258)+TX, metam (519)+TX, metam-potassium (519)+TX, metam-sodium (519)+TX, methyl bromide (537)+TX, methyl isothiocyanate (543)+TX, milbemycin oxime [CCN]+TX, moxidectin [CCN]+TX, Myrothecium verrucaria composition (565)+TX, NC-184 (compound code)+TX, oxamyl (602)+TX, phorate (636)+TX, phosphamidon (639)+TX, phosphocarb [CCN]+TX, sebufos+TX, selamectin [CCN]+TX, spinosad (737)+TX, terbam+TX, terbufos (773)+TX, tetrachlorothiophene (IUPAC/Chemical Abstracts name) (1422)+TX, thiafenox+TX, thionazin (1434)+TX, triazophos (820)+TX, triazuron+TX, xylenols [CCN]+TX, YI-5302 (compound code) and zeatin (210)+TX, fluensulfone [318290-98-1]+TX,

a nitrification inhibitor selected from the group of substances consisting of potassium ethylxanthate [CCN] and nitrapyrin (580)+TX,

a plant activator selected from the group of substances consisting of acibenzolar (6)+TX, acibenzolar-S-methyl (6)+TX, probenazole (658) and Reynoutria sachalinensis extract (720)+TX, a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,

a virucide selected from the group of substances consisting of imanin [CCN] and ribavirin [CCN]+TX,

a wound protectant selected from the group of substances consisting of mercuric oxide (512)+TX, octhilinone (590) and thiophanate-methyl (802)+TX, and biologically active compounds selected from the group consisting of azaconazole (60207-31-0]+TX, benzovindiflupyr [1072957-71-1]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol 25 [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl 30 [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzolar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-L190 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, oxathiapiprolin [1003318-67-9]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yI)-amide (dislosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyrid i nyI)-2H,11H naphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX; lancotrione [1486617-21-3]+TX, florpyrauxifen [943832-81-3]]+TX, ipfentrifluconazole [1417782-08-1]+TX, mefentrifluconazole [1417782-03-6]+TX, quinofumelin [861647-84-9]]+TX, chloroprallethrin [399572-87-3]]+TX, cyhalodiamide [1262605-53-7]]+TX, fluazaindolizine [1254304-22-7]+TX, fluxametamide [928783-29-3]+TX, epsilon-metofluthrin [240494-71-7]]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1] and kappa-tefluthrin [391634-71-2]+TX; and

microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD#32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield 1-1C®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus; and

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakelie®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®); and

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CMO)+TX, (E+TX, Z+TX, Z)-3+TX,8+TX,11 Tetradecatrienyl acetate+TX, (Z+TX, Z+TX, E)-7+TX,11+TX,13-Hexadecatrienal+TX, (E+TX, Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and

Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adeline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius califomicus (Amblyline®+TX, Spical®)+TX, Amblyseius cucumeris (Thripex®+TX, Bugline Cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripoli®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys+TX, BioNem+TX, Exhibitline Hm®+TX, Larvanem-M®)+TX, Hippodamia convergens+TX, Hypoaspis aculeifer (Aculeifer-System®+TX, Entomite-A®)+TX, Hypoaspis miles (Hypoline M®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus califomicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline+TX, Orius laevigatus (Thripor-L®+TX, Oriline I®)+TX, Orius majusculus (Oriline M®)+TX, Orius strigicoffis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline Sf®+TX, Scia-Rid®+TX, Entonem®)+TX, Steinernema kraussei (Nemasys L®+TX, BioNem L®+TX, Exhibitline Srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; and

other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®+TX, Ni-HIBIT Gold CST®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (SD-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula (I) selected from a compound described in one of Tables 1A to 18A, 1B to 18B, 1C to 18C (below), or Tables T1, T2 or T3 (below), and an active ingredient as described above are preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from one of Tables 1A to 18A, 1B to 18B, 1C to 18C (below), or Tables T1, T2 or T3 (below), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Tables 1A to 18A, 1B to 18B, 1C to 18C (below), or Tables T1, T2 or T3 (below), and the active ingredient(s) as described above, is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of invention is related to the use of a compound of formula (I) or of a preferred individual compound as defined herein, of a composition comprising at least one compound of formula (I) or at least one preferred individual compound as above-defined, or of a fungicidal or insecticidal mixture comprising at least one compound of formula (I) or at least one preferred individual compound as above-defined, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of invention is related to a method of controlling or preventing an infestation of plants, e.g., useful plants such as crop plants, propagation material thereof, e.g. seeds, harvested crops, e.g., harvested food crops, or of non-living materials by insects or by phytopathogenic or spoilage microorganisms or organisms potentially harmful to man, especially fungal organisms, which comprises the application of a compound of formula (I) or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I), or an agrochemical composition which contains at least one of said compounds, is foliar application. The frequency of application and the rate of application will depend on the risk of infestation by the corresponding pathogen or insect. However, the compounds of formula (I) can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, e.g. a composition containing the compound of formula (I), and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of formula I per kg of seed, preferably from 0.01 to 10 g per kg of seed are generally sufficient.

Suitably, a composition comprising a compound of formula (I) according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the ondensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

### Table 1A:

This table discloses 72 specific compounds of the formula (T-1A) according to the invention:

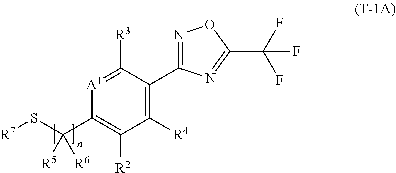

wherein A¹ is C—R¹ and R¹, R², R³ and R⁴ are hydrogen, n is 0, and R⁷ is as defined below in the Table A.

Each of Tables 2A to 18A (which follow Table 1A) make available 72 individual compounds of the formula (T-1A) in which A¹, R¹, R², R³, R⁴, R⁵, and R⁶ are as specifically defined in Tables 2A to 18A, which refer to Table A wherein R⁷ is specifically defined.

TABLE A

| Compound no. | R⁷ |
|---|---|
| 1.001 | propyl |
| 1.002 | isopropyl |
| 1.003 | butyl |
| 1.004 | tert-butyl |
| 1.005 | 1,3-dimethylbutyl |
| 1.006 | 2-ethylbutyl |
| 1.007 | 2-methylbutyl |
| 1.008 | sec-butyl |
| 1.009 | pentyl |
| 1.010 | isopentyl |
| 1.011 | hexyl |
| 1.012 | 2,2,2-trifluoroethyl |
| 1.013 | 3,3,3-trifluoropropyl |
| 1.014 | 4,4,4-trifluorobutyl |
| 1.015 | 2-methoxyethyl |
| 1.016 | 2-ethoxyethyl |
| 1.017 | 3-methoxypropyl |
| 1.018 | 3-ethoxypropyl |
| 1.019 | cyclopropyl |
| 1.020 | cyclobutyl |
| 1.021 | cyclopentyl |
| 1.022 | cyclohexyl |
| 1.023 | cycloheptanyl |
| 1.024 | cyclopentylmethyl |
| 1.025 | cyclohexylmethyl |
| 1.026 | phenyl |
| 1.027 | o-tolyl |
| 1.028 | m-tolyl |
| 1.029 | p-tolyl |
| 1.030 | 2-methoxyphenyl |
| 1.031 | 3-methoxyphenyl |
| 1.032 | 4-methoxyphenyl |
| 1.033 | 2-fluorophenyl |
| 1.034 | 3-fluorophenyl |
| 1.035 | 4-fluorophenyl |
| 1.036 | 2-chlorophenyl |
| 1.037 | 3-chlorophenyl |
| 1.038 | 4-chlorophenyl |
| 1.039 | 4-trifluoromethylphenyl |
| 1.040 | 2,4-difluorophenyl |
| 1.041 | 2,5-difluorophenyl |
| 1.042 | 2,6-difluorophenyl |
| 1.043 | 3,4-difluorophenyl |
| 1.044 | 3,5-difluorophenyl |
| 1.045 | 3,5-dichlorophenyl |
| 1.046 | 2,4-dimethylphenyl |
| 1.047 | 2,5-dimethylphenyl |
| 1.048 | 3,4-dimethylphenyl |
| 1.049 | 4-fluoro-2-methyl-phenyl |
| 1.050 | 4-fluoro-3-methyl-phenyl |
| 1.051 | 3-fluoro-4-methyl-phenyl |
| 1.052 | 2-fluoro-4-methoxy-phenyl |
| 1.053 | benzyl |
| 1.054 | o-tolylmethyl |
| 1.055 | m-tolylmethyl |
| 1.056 | p-tolylmethyl |
| 1.057 | 2-methoxyphenylmethyl |
| 1.058 | 3-methoxyphenylmethyl |
| 1.059 | 4-methoxyphenylmethyl |
| 1.060 | 2-fluorophenylmethyl |
| 1.061 | 3-fluorophenylmethyl |
| 1.062 | 4-fluorophenylmethyl |
| 1.063 | 2,4-difluorophenylmethyl |
| 1.064 | 2,5-difluorophenylmethyl |
| 1.065 | 2,6-difluorophenylmethyl |
| 1.066 | 3,4-difluorophenylmethyl |
| 1.067 | 3,5-difluorophenylmethyl |
| 1.068 | 2,4-dimethylphenylmethyl |
| 1.069 | 2,5-dimethylphenylmethyl |
| 1.070 | 2-phenylethyl |
| 1.071 | 2-4-methoxyphenylethyl |
| 1.072 | 2-p-tolylethyl |

### Table 2A:

This table discloses 72 specific compounds of formula (T-1A) wherein A¹ is C—R¹ and R², R³ and R⁴ are hydrogen, R¹ is fluorine, n is 0, and R⁷ is as defined above in Table A.

### Table 3A:

This table discloses 72 specific compounds of formula (T-1A) wherein A¹ is C—R¹ and R², R³ and R⁴ are hydrogen, R¹ is chlorine, n is 0, and R⁷ is as defined above in Table A.

### Table 4A:

This table discloses 72 specific compounds of formula (T-1A) wherein A¹ is C—R¹ and R², R³ and R⁴ are hydrogen, R¹ is methyl, n is 0, and R⁷ is as defined above in Table A.

### Table 5A:

This table discloses 72 specific compounds of formula (T-1A) wherein A¹ is C—R¹ and R², R³ and R⁴ are hydrogen, R¹ is trifluoromethyl, n is 0, and R⁷ is as defined above in Table A.

### Table 6A:

This table discloses 72 specific compounds of formula (T-1A) wherein A¹ is N and R², R³, and R⁴ are hydrogen, n is 0, and R⁷ is as defined above in Table A.

### Table 7A:

This table discloses 72 specific compounds of formula (T-1A) wherein A¹ is C—R¹ and R¹, R², and R⁴ are hydrogen, R³ is fluorine, n is 0, and R⁷ is as defined above in Table A.

### Table 8A:

This table discloses 72 specific compounds of formula (T-1A) wherein A¹ is C—R¹ and R¹, R², R³, R⁴, R⁵ and R⁶ are hydrogen, n is 1, and R⁷ is as defined above in Table A.

### Table 9A:

This table discloses 72 specific compounds of formula (T-1A) wherein A¹ is C—R¹ and R², R³, R⁴, R⁵ and R⁶ are hydrogen, R¹ is fluorine, n is 1, and R⁷ is as defined above in Table A.

### Table 10A:

This table discloses 72 specific compounds of formula (T-1A) wherein A¹ is C—R¹ and R², R³, R⁴, R⁵ and R⁶ are hydrogen, R¹ is chlorine, n is 1, and R⁷ is as defined above in Table A.

### Table 11A:

This table discloses 72 specific compounds of formula (T-1A) wherein A¹ is C—R¹ and R², R³, R⁴, R⁵ and R⁶ are hydrogen, R¹ is methyl, n is 1, and R⁷ is as defined above in Table A.

### Table 12A:

This table discloses 72 specific compounds of formula (T-1A) wherein A¹ is C—R¹ and R², R³, R⁴, R⁵ and R⁶ are hydrogen, R¹ is trifluoromethyl, n is 1, and R⁷ is as defined above in Table A.

### Table 13A:

This table discloses 72 specific compounds of formula (T-1A) wherein A¹ is C—R¹ and R², R³, R⁴, R⁵ and R⁶ are hydrogen, R¹ is methoxy, n is 1, and R⁷ is as defined above in Table A.

### Table 14A:

This table discloses 72 specific compounds of formula (T-1A) wherein A¹ is N and R², R³, R⁴, R⁵ and R⁶ are hydrogen, n is 1, and R⁷ is as defined above in Table A.

### Table 15A:

This table discloses 72 specific compounds of formula (T-1A) wherein A¹ is C—R¹ and R¹, R² and R⁴ are hydrogen, R³ is fluorine, n is 0, and R⁷ is as defined above in Table A.

### Table 16A:

This table discloses 72 specific compounds of formula (T-1A) wherein A¹ is C—R¹ and R¹, R², R³, R⁴ and R⁵ are hydrogen, R⁶ is methyl, n is 1, and R⁷ is as defined above in Table A.

### Table 17A:

This table discloses 72 specific compounds of formula (T-1A) wherein A¹ is C—R¹ and R³, R⁴, R⁵ and R⁶ are hydrogen, R¹ and R² are fluorine, n is 1, and R⁷ is as defined above in Table A.

### Table 18A:

This table discloses 72 specific compounds of formula (T-1A) wherein A¹ is C—R¹ and R², R⁴, R⁵ and R⁶ are hydrogen, R¹ and R³ are fluorine, n is 1, and R⁷ is as defined above in Table A.

### Table 1B:

This table discloses 72 specific compounds of the formula (T-1B):

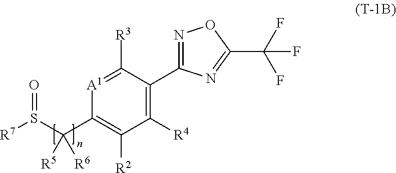

wherein A¹ is C—R¹ and R¹, R², R³ and R⁴ are hydrogen, n is 0, and R⁷ is as defined below in Table B.

Each of Tables 2B to 18B (which follow Table 1B) make available 72 individual compounds of the formula (T-1B) in which A¹, R¹, R², R³, R⁴, R⁵, and R⁶ are as specifically defined in Tables 2B to 18B, which refer to Table B wherein R⁷ is specifically defined.

TABLE B

| Compound no. | R⁷ |
|---|---|
| 2.001 | propyl |
| 2.002 | isopropyl |
| 2.003 | butyl |
| 2.004 | tert-butyl |
| 2.005 | 1,3-dimethylbutyl |
| 2.006 | 2-ethylbutyl |
| 2.007 | 2-methylbutyl |
| 2.008 | sec-butyl |
| 2.009 | pentyl |
| 2.010 | isopentyl |
| 2.011 | hexyl |
| 2.012 | 2,2,2-trifluoroethyl |
| 2.013 | 3,3,3-trifluoropropyl |
| 2.014 | 4,4,4-trifluorobutyl |
| 2.015 | 2-methoxyethyl |
| 2.016 | 2-ethoxyethyl |
| 2.017 | 3-methoxypropyl |
| 2.018 | 3-ethoxypropyl |
| 2.019 | cyclopropyl |
| 2.020 | cyclobutyl |
| 2.021 | cyclopentyl |
| 2.022 | cyclohexyl |
| 2.023 | cycloheptanyl |
| 2.024 | cyclopentylmethyl |
| 2.025 | cyclohexylmethyl |
| 2.026 | phenyl |
| 2.027 | o-tolyl |
| 2.028 | m-tolyl |
| 2.029 | p-tolyl |
| 2.030 | 2-methoxyphenyl |
| 2.031 | 3-methoxyphenyl |
| 2.032 | 4-methoxyphenyl |
| 2.033 | 2-fluorophenyl |
| 2.034 | 3-fluorophenyl |
| 2.035 | 4-fluorophenyl |
| 2.036 | 2-chlorophenyl |
| 2.037 | 3-chlorophenyl |
| 2.038 | 4-chlorophenyl |
| 2.039 | 4-trifluoromethylphenyl |
| 2.040 | 2,4-difluorophenyl |
| 2.041 | 2,5-difluorophenyl |
| 2.042 | 2,6-difluorophenyl |
| 2.043 | 3,4-difluorophenyl |
| 2.044 | 3,5-difluorophenyl |
| 2.045 | 3,5-dichlorophenyl |
| 2.046 | 2,4-dimethylphenyl |
| 2.047 | 2,5-dimethylphenyl |
| 2.048 | 3,4-dimethylphenyl |
| 2.049 | 4-fluoro-2-methyl-phenyl |
| 2.050 | 4-fluoro-3-methyl-phenyl |
| 2.051 | 3-fluoro-4-methyl-phenyl |
| 2.052 | 2-fluoro-4-methoxy-phenyl |
| 2.053 | benzyl |
| 2.054 | o-tolylmethyl |
| 2.055 | m-tolylmethyl |
| 2.056 | p-tolylmethyl |
| 2.057 | 2-methoxyphenylmethyl |
| 2.058 | 3-methoxyphenylmethyl |
| 2.059 | 4-methoxyphenylmethyl |
| 2.060 | 2-fluorophenylmethyl |
| 2.061 | 3-fluorophenylmethyl |
| 2.062 | 4-fluorophenylmethyl |
| 2.063 | 2,4-difluorophenylmethyl |
| 2.064 | 2,5-difluorophenylmethyl |
| 2.065 | 2,6-difluorophenylmethyl |
| 2.066 | 3,4-difluorophenylmethyl |
| 2.067 | 3,5-difluorophenylmethyl |
| 2.068 | 2,4-dimethylphenylmethyl |
| 2.069 | 2,5-dimethylphenylmethyl |
| 2.070 | 2-phenylethyl |
| 2.071 | 2-4-methoxyphenylethyl |
| 2.072 | 2-p-tolylethyl |

### Table 2B:

This table discloses 72 specific compounds of formula (T-1B) wherein A¹ is C—R¹ and R², R³ and R⁴ are hydrogen, R¹ is fluorine, n is 0, and R⁷ is as defined above in Table B

### Table 3B:

This table discloses 72 specific compounds of formula (T-1B) wherein A¹ is C—R¹ and R², R³ and R⁴ are hydrogen, R¹ is chlorine, n is 0, and R⁷ is as defined above in Table B.

### Table 4B:

This table discloses 72 specific compounds of formula (T-1B) wherein A¹ is C—R¹ and R², R³ and R⁴ are hydrogen, R¹ is methyl, n is 0, and R⁷ is as defined above in Table B.

### Table 5B:

This table discloses 72 specific compounds of formula (T-1B) wherein A¹ is C—R¹ and R², R³ and R⁴ are hydrogen, R¹ is trifluoromethyl, n is 0, and R⁷ is as defined above in Table B.

### Table 6B:

This table discloses 72 specific compounds of formula (T-1B) wherein A¹ is N and R², R³ and R⁴ are hydrogen, n is 0, and R⁷ is as defined above in Table B.

### Table 7B:

This table discloses 72 specific compounds of formula (T-1B) wherein A¹ is C—R¹ and R¹, R² and R⁴ are hydrogen, R³ is fluorine, n is 0, and R⁷ is as defined above in Table B.

### Table 8B:

This table discloses 72 specific compounds of formula (T-1B) wherein A¹ is C—R¹ and R¹, R², R³, R⁴, R⁵ and R⁶ are hydrogen, n is 1, and R⁷ is as defined above in Table B.

### Table 9B:

This table discloses 72 specific compounds of formula (T-1B) wherein A¹ is C—R¹ and R², R³, R⁵ and R⁶ are hydrogen, R¹ is fluorine, n is 1, and R⁷ is as defined above in Table B.

### Table 10B:

This table discloses 72 specific compounds of formula (T-1B) wherein A¹ is C—R¹ and R¹, R², R³, R⁴, R⁵ and R⁶ are hydrogen, R¹ is chlorine, n is 1, and R⁷ is as defined above in Table B.

### Table 11B:

This table discloses 72 specific compounds of formula (T-1B) wherein A¹ is C—R¹ and R², R³, R⁴, R⁵ and R⁶ are hydrogen, R¹ is methyl, n is 1, and R⁷ is as defined above in Table B.

### Table 12B:

This table discloses 72 specific compounds of formula (T-1B) wherein A¹ is C—R¹ and R², R³, R⁴, R⁵ and R⁶ are hydrogen, R¹ is trifluoromethyl, n is 1, and R⁷ is as defined above in Table B.

### Table 13B:

This table discloses 72 specific compounds of formula (T-1B) wherein A¹ is C—R¹ and R², R³, R⁴, R⁵ and R⁶ are hydrogen, R¹ is methoxy, n is 1, and R⁷ is as defined above in Table B.

### Table 14B:

This table discloses 72 specific compounds of formula (T-1B) wherein A¹ is N and R², R³, R⁴, R⁵ and R⁶ are hydrogen, n is 1, and R⁷ is as defined above in Table B.

### Table 15B:

This table discloses 72 specific compounds of formula (T-1B) wherein A¹ is C—R¹and R¹, R², and R⁴ are hydrogen, R³ is fluorine, n is 0, and R⁷ is as defined above in Table B.

### Table 16B:

This table discloses 72 specific compounds of formula (T-1B) wherein A¹ is C—R¹and R¹, R², R³, R⁴, and R⁵ are hydrogen, R⁶ is methyl, n is 1, and R⁷ is as defined above in Table B.

### Table 17B:

This table discloses 72 specific compounds of formula (T-1B) wherein A¹ is C—R¹ and R³, R⁴, R⁵ and R⁶ are hydrogen, R¹ and R² are fluorine, n is 1, and R⁷ is as defined above in Table B.

### Table 18B:

This table discloses 72 specific compounds of formula (T-1B) wherein A¹ is C—R¹ and R², R⁴, R⁵ and R⁶ are hydrogen, R¹ and R³ are fluorine, n is 1, and R⁷ is as defined above in Table B.

### Table 1C:

This table discloses 72 specific compounds of the formula (T-1C):

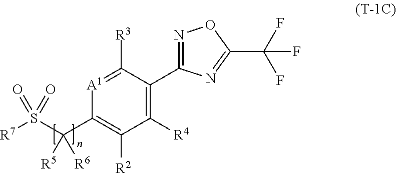

wherein A¹ is C—R¹ and R¹, R², R³ and R⁴ are hydrogen, n is 0, and R⁷ is as defined below in Table C.

Each of Tables 2C to 17C (which follow Table 1C) make available 72 individual compounds of the formula (T-1C) in which A¹, R¹, R², R³, R⁴, R⁵, and R⁶ are as specifically defined in Tables 2C to 18C, which refer to Table C wherein R⁷ is specifically defined.

TABLE C

| Compound no. | R⁷ |
|---|---|
| 3.001 | propyl |
| 3.002 | isopropyl |
| 3.003 | butyl |
| 3.004 | tert-butyl |
| 3.005 | 1,3-dimethylbutyl |
| 3.006 | 2-ethylbutyl |
| 3.007 | 2-methylbutyl |
| 3.008 | sec-butyl |
| 3.009 | pentyl |
| 3.010 | isopentyl |
| 3.011 | hexyl |
| 3.012 | 2,2,2-trifluoroethyl |
| 3.013 | 3,3,3-trifluoropropyl |
| 3.014 | 4,4,4-trifluorobutyl |
| 3.015 | 2-methoxyethyl |
| 3.016 | 2-ethoxyethyl |
| 3.017 | 3-methoxypropyl |
| 3.018 | 3-ethoxypropyl |
| 3.019 | cyclopropyl |
| 3.020 | cyclobutyl |
| 3.021 | cyclopentyl |
| 3.022 | cyclohexyl |
| 3.023 | cycloheptanyl |
| 3.024 | cyclopentylmethyl |
| 3.025 | cyclohexylmethyl |
| 3.026 | phenyl |
| 3.027 | o-tolyl |
| 3.028 | m-tolyl |
| 3.029 | p-tolyl |
| 3.030 | 2-methoxyphenyl |
| 3.031 | 3-methoxyphenyl |
| 3.032 | 4-methoxyphenyl |
| 3.033 | 2-fluorophenyl |
| 3.034 | 3-fluorophenyl |
| 3.035 | 4-fluorophenyl |
| 3.036 | 2-chlorophenyl |
| 3.037 | 3-chlorophenyl |
| 3.038 | 4-chlorophenyl |
| 3.039 | 4-trifluoromethylphenyl |
| 3.040 | 2,4-difluorophenyl |
| 3.041 | 2,5-difluorophenyl |
| 3.042 | 2,6-difluorophenyl |
| 3.043 | 3,4-difluorophenyl |
| 3.044 | 3,5-difluorophenyl |
| 3.045 | 3,5-dichlorophenyl |
| 3.046 | 2,4-dimethylphenyl |
| 3.047 | 2,5-dimethylphenyl |
| 3.048 | 3,4-dimethylphenyl |
| 3.049 | 4-fluoro-2-methyl-phenyl |
| 3.050 | 4-fluoro-3-methyl-phenyl |
| 3.051 | 3-fluoro-4-methyl-phenyl |
| 3.052 | 2-fluoro-4-methoxy-phenyl |
| 3.053 | benzyl |
| 3.054 | o-tolylmethyl |
| 3.055 | m-tolylmethyl |
| 3.056 | p-tolylmethyl |
| 3.057 | 2-methoxyphenylmethyl |
| 3.058 | 3-methoxyphenylmethyl |
| 3.059 | 4-methoxyphenylmethyl |
| 3.060 | 2-fluorophenylmethyl |
| 3.061 | 3-fluorophenylmethyl |
| 3.062 | 4-fluorophenylmethyl |
| 3.063 | 2,4-difluorophenylmethyl |
| 3.064 | 2,5-difluorophenylmethyl |
| 3.065 | 2,6-difluorophenylmethyl |
| 3.066 | 3,4-difluorophenylmethyl |
| 3.067 | 3,5-difluorophenylmethyl |
| 3.068 | 2,4-dimethylphenylmethyl |
| 3.069 | 2,5-dimethylphenylmethyl |
| 3.070 | 2-phenylethyl |
| 3.071 | 2-4-methoxyphenylethyl |
| 3.072 | 2-p-tolylethyl |

### Table 2C:

This table discloses 72 specific compounds of formula (T-1C) wherein A¹ is C—R¹ and R², R³ and R⁴ are hydrogen, R¹ is fluorine, n is 0, and R⁷ is as defined above in Table C.

### Table 3C:

This table discloses 72 specific compounds of formula (T-1C) wherein A¹ is C—R¹ and R², R³ and R⁴ are hydrogen, R¹ is chlorine, n is 0, and R⁷ is as defined above in Table C.

### Table 4C:

This table discloses 72 specific compounds of formula (T-1C) wherein A¹ is C—R¹ and R², R³ and R⁴ are hydrogen, R¹ is methyl, n is 0, and R⁷ is as defined above in Table C.

### Table 5C:

This table discloses 72 specific compounds of formula (T-1C) wherein A¹ is C—R¹ and R², R³ and R⁴ are hydrogen, R¹ is trifluoromethyl, n is 0, and R⁷ is as defined above in Table C.

### Table 6C:

This table discloses 72 specific compounds of formula (T-1C) wherein A¹ is N and R², R³, and R⁴ are hydrogen, n is 0, and R⁷ is as defined above in Table C.

### Table 7C:

This table discloses 72 specific compounds of formula (T-1C) wherein A¹ is C—R¹ and R¹, R², and R⁴ are hydrogen, R³ is fluorine, n is 0, and R⁷ is as defined above in Table C.

### Table 8C:

This table discloses 72 specific compounds of formula (T-1C) wherein A¹ is C—R¹ and R¹, R², R³, R⁴, R⁵ and R⁶ are hydrogen, n is 1, and R⁷ is as defined above in Table C.

### Table 9C:

This table discloses 72 specific compounds of formula (T-1C) wherein A¹ is C—R¹ and R², R³, R⁵ and R⁶ are hydrogen, R¹ is fluorine, n is 1, and R⁷ is as defined above in Table C.

### Table 10C:

This table discloses 72 specific compounds of formula (T-1C) wherein A¹ is C—R¹ and R², R³, R⁴, Wand R⁶ are hydrogen, R¹ is chlorine, n is 1, and R⁷ is as defined above in Table C.

### Table 11C:

This table discloses 72 specific compounds of formula (T-1C) wherein A¹ is C—R¹ and R², R³, R⁴, Wand R⁶ are hydrogen, R¹ is methyl, n is 1, and R⁷ is as defined above in Table C.

### Table 12C:

This table discloses 72 specific compounds of formula (T-1C) wherein A¹ is C—R¹ and R², R³, R⁴, Wand R⁶ are hydrogen, R¹ is trifluoromethyl, n is 1, and R⁷ is as defined above in Table C.

### Table 13C:

This table discloses 72 specific compounds of formula (T-1C) wherein A¹ is C—R¹ and R², R³, R⁴, Wand R⁶ are hydrogen, R¹ is methoxy, n is 1, and R⁷ is as defined above in Table C.

### Table 14C:

This table discloses 72 specific compounds of formula (T-1C) wherein A¹ is N and R², R³, R⁴, R⁵and R⁶ are hydrogen, n is 1, and R⁷ is as defined above in Table C.

### Table 15C:

This table discloses 72 specific compounds of formula (T-1C) wherein A¹ is C—R¹and R¹, R², and R⁴ are hydrogen, R³ is fluorine, n is 0, and R⁷ is as defined above in Table C.

### Table 16C:

This table discloses 72 specific compounds of formula (T-1C) wherein A¹ is C—R¹ and R¹, R², R³, R⁴, and R⁵ are hydrogen, R⁶ is methyl, n is 1, and R⁷ is as defined above in Table C.

### Table 17C:

This table discloses 72 specific compounds of formula (T-1C) wherein A¹ is C—R¹ and R³, R⁴, R⁵ and R⁶ are hydrogen, R¹ and R² are fluorine, n is 1, and R⁷ is as defined above in Table C.

### Table 18C:

This table discloses 72 specific compounds of formula (T-1C) wherein A¹ is C—R¹ and R², R⁴, R⁵ and R⁶ are hydrogen, R¹ and R³ are fluorine, n is 1, and R⁷ is as defined above in Table C.

### EXAMPLES

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, δ ppm, 3 ppm, 1.5 ppm, 0.8 or 0.2 ppm.

Compounds of Formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius (° C.) and “mp.” means melting point. LC/MS means Liquid Chromatography Mass Spectrometry and the description of the apparatus and the method is as follows:

### The LC/MS Apparatus and Method (Method A) is:

SQ Detector 2 from Waters
Ionisation method: Electrospray
Polarity: positive and negative ions

### Capillary (kV) 3.0, Cone (V) 30.00, Extractor (V) 2.00, Source Temperature (° C.) 150, Desolvation

### Temperature (° C.) 350, Cone Gas Flow (L/Hr) 0, Desolvation Gas Flow (L/Hr) 650

Mass range: 100 to 900 Da
DAD Wavelength range (nm): 210 to 500
Method Waters ACQUITY UPLC with the following HPLC gradient conditions
(Solvent A: Water/Methanol 20:1+0.05% formic acid and Solvent B: Acetonitrile+0.05% formic acid)

| Time (minutes) | A (%) | B (%) | Flow rate (ml/min) |
|---|---|---|---|
| 0 | 100 | 0 | 0.85 |
| 1.2 | 0 | 100 | 0.85 |
| 1.5 | 0 | 100 | 0.85 |

Type of column: Waters ACQUITY UPLC HSS T3; Column length: 30 mm; Internal diameter of column: 2.1 mm; Particle Size: 1.8 micron; Temperature: 60° C.

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

### Emulsifiable Concentrate

| | |
|---|---|
| active ingredient [compound of formula (1)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| Talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

### Extruder Granules

| | |
|---|---|
| Active ingredient [compound of formula (I)] | 15% |
| sodium lignosulfonate | 2% |
| Carboxymethylcellulose | 1% |
| Kaolin | 82% |

The active ingredient is mixed and ground with the adjuvants, and the mixture is moistened with water. The mixture is extruded and then dried in a stream of air.

### Coated Granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

### Suspension Concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| Carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow-Release Capsule Suspension

28 parts of a combination of the compound of formula I are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### LIST OF ABBREVIATIONS

AIBN=azobisisobutyronitrile
DMF=dimethylformamide
DIPEA=N,N-di-isopropylethylamine
EtOAc=ethyl acetate
HCl=hydrochloric acid
mp=melting point
° C.=degrees Celsius
MeOH=methyl alcohol
NaOH=sodium hydroxide
NBS=N-bromosuccinimide
min=minutes
rt=room temperature
TFAA=trifluoroacetic acid anhydride
THF=tetrahydrofuran
LC/MS=Liquid Chromatography Mass Spectrometry (description of the apparatus and the methods used for LC/MS analysis are given above)

### Preparation Examples

### Example 1: this Example Illustrates the Preparation of 3-[4-[(2-fluorophenyl)sulfanylmethyl]phenyl]-5-(trifluoromethyl)-1,2,4-oxadiazole (Compound 1.15 of Table T1)

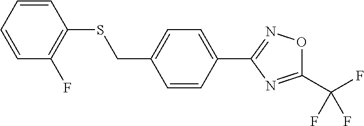 [SMILES: Fc1ccccc1SCc1ccc(-c2noc(C(F)(F)F)n2)cc1]

### Step 1: Preparation of N′-hydroxy-4-methyl-benzamidine

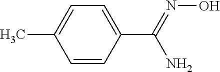

To a suspension of 4-methylbenzonitrile (35.0 g, 0.29 mol) in ethanol (220 mL) and water (440 mL) was added at rt hydroxylamine hydrochloride (41.1 g, 0.58 mol), potassium carbonate (65.4 g, 0.47 mol) and 8-hydroxyquinoline (0.22 g, 1.5 mmol). The reaction mixture was heated at 80° C. for 4 hours. The mixture was cooled to rt and diluted with 2N HCl until pH 8. Ethanol was evaporated under reduced pressure. The mixture was filtered, washed with water and dried under vacuum to afford 39.1 g of N′-hydroxy-4-methyl-benzamidine as a pale gum then used directly in the next preparation step without further purification. LC/MS retention time=0.23 minutes, 151.0 (M+H).

### Step 2: Preparation of 3-(p-tolyl)-5-(trifluoromethyl)-1,2,4-oxadiazole

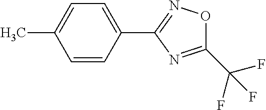 [SMILES: Cc1ccc(-c2noc(C(F)(F)F)n2)cc1]

To a solution of N′-hydroxy-4-methyl-benzamidine (38.7 g, 0.25 mol) in 2-methyltetrahydrofuran (750 mL) was added TFAA at 0° C. The reaction mixture was stirred at 15° C. for two hours and diluted with water. The organic layers were separated, washed successively with sodium bicarbonate solution, saturated aqueous ammonium chloride solution and water, then dried over sodium sulfate, filtered and evaporated to dryness. The crude residue was subject to flash chromatography over silica gel (750 g pre-packed column) with heptane/EtOAc (99:1 to 90:10) to afford 54.1 g of the title compound as a clear oil, which solidified after storage.

LC/MS retention time=1.15 minutes, mass not detected.

¹H NMR (400 MHz, CDCl₃) δ ppm: 8.00 (d, 2H), 7.32 (d, 2H), 2.45 (s, 3H).

¹⁹F NMR (376 MHz, CDCl₃) δ ppm: −65.41 (s).

### Step 3a: Preparation of 3-[4-(bromomethyl)phenyl]-5-(trifluoromethyl)-1,2,4-oxadiazole

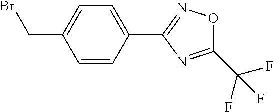 [SMILES: FC(F)(F)c1nc(-c2ccc(CBr)cc2)no1]

A mixture of 3-(p-tolyl)-5-(trifluoromethyl)-1,2,4-oxadiazole (56.0 g, 0.24 mol) and NBS (45.4 g, 0.25 mol) in tetrachloromethane (480 mL) under argon was heated to 70° C. AIBN (4.03 g, 24 mmol) was added and the reaction mixture stirred at 65° C. for 18 hours. The mixture was cooled to rt, diluted with dichloromethane and water, and the layers were separated. The organic phase was washed with sodium bicarbonate solution, dried over sodium sulfate, filtered and evaporated to dryness. The crude residue was subjected to flash chromatography over silica gel (750 g pre-packed column) with cyclohehane/EtOAc 100:0 to 95:5 to afford 44.7 g of the title compound as a white solid mp: 58-63° C.

¹H NMR (400 MHz, CDCl₃) δ ppm: 8.11 (d, 2H), 7.55 (d, 2H), 4.53 (s, 2H).

¹⁹F NMR (400 MHz, CDCl₃) δ ppm: −65.32 (s).

A by-product, 3-[4-(dibromomethyl)phenyl]-5-(trifluoromethyl)-1,2,4-oxadiazole, was isolated as a white solid mp: 61-66° C.

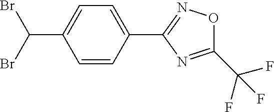 [SMILES: FC(F)(F)c1nc(-c2ccc(C(Br)Br)cc2)no1]

¹H NMR (400 MHz, CDCl₃) δ ppm: 8.15 (d, 2H), 7.73 (d, 2H), 6.68 (s, 1H).

¹⁹F NMR (376 MHz, CDCl₃) δ ppm: −65.34 (s).

### Step 3b: Preparation of 3-[4-(bromomethyl)phenyl]-5-(trifluoromethyl)-1,2,4-oxadiazole

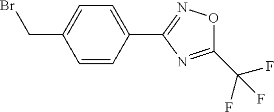 [SMILES: FC(F)(F)c1nc(-c2ccc(CBr)cc2)no1]

To a 1:9 ratio mixture of 3-[4-(bromomethyl)phenyl]-5-(trifluoromethyl)-1,2,4-oxadiazole and 3-[4-(dibromomethyl)phenyl]-5-(trifluoromethyl)-1,2,4-oxadiazole (10.2 g) in acetonitrile (95 mL), water (1.9 mL) and DIPEA (6.20 ml, 35.7 mmol) was added diethylphosphite (4.7 ml, 35.7 mmol) at 5° C. The mixture was stirred at 5-10° C. for two hours, water and 1M HCl added and acetonitrile evaporated under reduced pressure. The white slurry was extracted three times with dichloromethane. The combined organic layers were dried over sodium sulfate, and filtered. The solvent was removed under reduced pressure and the resultant crude residue subjected to flash chromatography over silica gel (40 g pre-packed column) with cyclohexane/EtOAc 99:1 to 9:1 to afford 7.10 g of 3-[4-(bromomethyl)phenyl]-5-(trifluoromethyl)-1,2,4-oxadiazole.

¹H NMR (400 MHz, CDCl₃) δ ppm: 8.11 (d, 2H), 7.55 (d, 2H), 4.53 (s, 2H).

¹⁹F NMR (376 MHz, CDCl₃) δ ppm: −65.32 (s).

### Step 4: Preparation of 3-[4-[(2-fluorophenyl)sulfanylmethyl]phenyl]-5-(trifluoromethyl)-1,2,4-oxadiazole

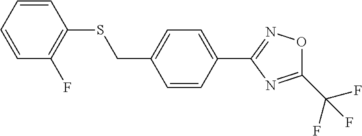 [SMILES: Fc1ccccc1SCc1ccc(-c2noc(C(F)(F)F)n2)cc1]

To a solution of 3-[4-(bromomethyl)phenyl]-5-(trifluoromethyl)-1,2,4-oxadiazole (200 mg, 0.63 mmol) in dichloromethane (1.3 mL) at 25° C. was added 2-fluorobenzenethiol (1.1 equiv, 0.74 mL, 0.69 mmol) and potassium carbonate (1.0 equiv, 0.09 g, 0.688 mmol). The reaction mixture was stirred for 12 hours then poured onto water and the layers were separated. The aqueous layer was extracted with dichloromethane (2×30 mL) and the combined organic layers washed with brine, dried over sodium sulfate, and filtered. The solvent was removed under reduced pressure and the resultant crude residue was purified by flash chromatography over silica gel (cyclohexane:EtOAc eluent gradient 1:0 to 1:1) to give 0.20 g (90% yield) of the title compound as a clear oil which solidified upon standing. LC/MS retention time=1.30 minutes, 355 (M+H); mp: 56-61° C.

¹H NMR (400 MHz, CDCl₃) δ ppm: 7.98 (d, 2H), 7.36 (d, 2H), 7.25 (m, 2H), 7.05 (m, 1H), 6.96 (m, 1H), 4.20 (s, 2H).

¹⁹F NMR (376 MHz, CDCl₃) δ ppm: −65.38 (s).

### Example 2: this Example Illustrates the Preparation of 3-[4-[(2-fluorophenyl)sulfinylmethyl]phenyl]-5-(trifluoromethyl)-1,2,4-oxadiazole (Compound 2.17 of Table T2) and 3-[4-[(2-fluorophenyl)sulfonylmethyl]phenyl]-5-(trifluoromethyl)-1,2,4-oxadiazole (Compound 3.11 of Table T3)

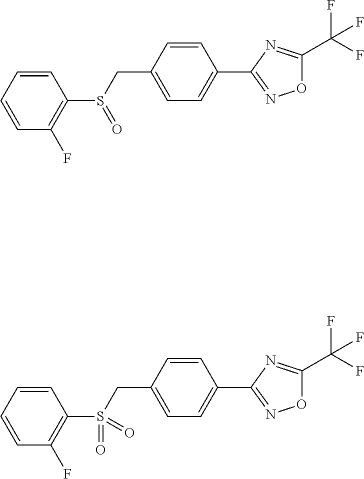 [SMILES: O=S(Cc1ccc(-c2noc(C(F)(F)F)n2)cc1)c1ccccc1F ; O=S(=O)(Cc1ccc(-c2noc(C(F)(F)F)n2)cc1)c1ccccc1F]

To a solution of 3-[4-[(2-fluorophenyl)sulfanylmethyl]phenyl]-5-(trifluoromethyl)-1,2,4-oxadiazole (175 mg, 0.82 mmol) in dichloromethane (7 mL) was added m-chloroperoxybenzoic acid (1.1 equiv, 93 mg, 0.54 mmol) at 25° C. After 2 hours, a second portion of m-chloroperoxybenzoic acid (0.5 equiv, 46 mg, 0.27 mmol) was introduced. After 30 minutes, a saturated aqueous solution of sodium bicarbonate was added and the aqueous layer extracted with dichloromethane (2×30 mL). The combined organic layers were washed with brine, dried over sodium sulfate, and then filtered. After the solvent was removed at reduced pressure, the crude residue was purified by flash chromatography over silica gel (cyclohexane:EtOAc eluent gradient 1:0 to 10:1). A first chromatography fraction afforded 0.092 g (51% yield) of compound 2.17 of Table T2 (3-[4-[(2-fluorophenyl)sulfinylmethyl]phenyl]-5-(trifluoromethyl)-1,2,4-oxadiazole) as a yellow solid [LC/MS retention time=1.12 minutes, 371 (M+1); mp: 109-117° C.] and a second chromatography fraction provided compound 3.11 of Table T3 (3-[4-[(2-fluorophenyl)sulfonylmethyl]phenyl]-5-(trifluoromethyl)-1,2,4-oxadiazole) as a yellow solid (0.070 g, 36% yield) [LC/MS retention time=1.1 minutes, 385 (M-1); mp: 150-154° C.].

[Compound 2.17 of Table T2]

¹H NMR (400 MHz, CDCl₃) δ ppm: 7.99 (m, 2H), 7.45 (m, 2H), 7.23 (m, 4H), 4.33 (d, 1H), 4.09 (d, 1H).

¹⁹F NMR (376 MHz, CDCl₃) δ ppm: −65.35 (s), −114.28 (s).

[Compound 3.11 of Table T3]

¹H NMR (400 MHz, CDCl₃) δ ppm: 8.03 (d, 2H), 7.61 (m, 2H), 7.39 (d, 2H), 7.21 (m, 2H), 4.60 (s, 2H)

¹⁹F NMR (376 MHz, CDCl₃) δ ppm: −65.36 (s), −114.28 (s).

### Example 3: this Example Illustrates the Preparation of 3-[4-(4-methoxyphenyl)sulfanylphenyl]-5-(trifluoromethyl)-1,2,4-oxadiazole (Compound 1.2 of Table T1)

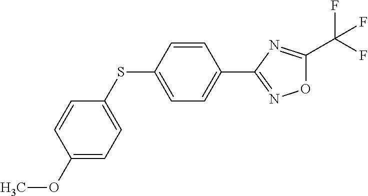 [SMILES: COc1ccc(Sc2ccc(-c3noc(C(F)(F)F)n3)cc2)cc1]

### Step 1: Preparation of 4-(4-methoxyphenyl)sulfanylbenzonitrile

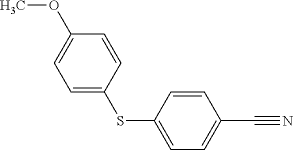 [SMILES: COc1ccc(Sc2ccc(C#N)cc2)cc1]

To a dry flask charged with sodium hydride (1.2 equiv., 2.57 mmol, 60 mass % NaH) and DMF (2 mL), 4-methoxythiophenol (0.300 g, 2.14 mmol) was introduced dropwise over 30 min during which gas evolution was observed. 4-Chlorobenzonitrile (1.1 equiv., 2.35 mmol) dissolved in DMF (1 mL) was introduced and the contents stirred for 1 hour at a temperature of 50° C. Upon reaction completion, the solution was quenched with water and extracted with ethyl acetate (2×50 mL). The organic layers were combined and dried over sodium sulfate, filtered, and concentrated at reduced pressure to produce an amorphous solid (512 mg, 98% yield, 90% purity) which was used without further purification. LC/MS retention time=1.08 minutes, 242 (M+H);

¹H NMR (400 MHz, CDCl₃) δ ppm: 7.40 (m, 4H), 7.06 (d, 2H), 6.96 (d, 2H), 3.85 (s, 3H).

### Step 2: Preparation of N-hydroxy-4-(4-methoxyphenyl)sulfanyl-benzamidine

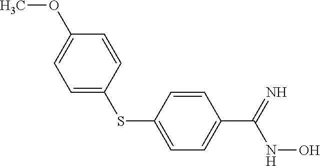 [SMILES: COc1ccc(Sc2ccc(C(=N)NO)cc2)cc1]

A solution of hydroxylamine hydrochloride (3.0 equiv, 0.44 g, 6.7 mmol) in water (20 mL) was added at rt to a stirred solution of 4-(4-methoxyphenyl)sulfanylbenzonitrile (512 mg, 2.13 mmol) in ethanol (7 mL), followed by dropwise addition of triethylamine (3.0 equiv, 0.88 mL, 6.7 mmol). The resulting suspension was heated at a temperature of 80° C. for 1 hour, cooled to 25° C., and concentrated under reduced pressure. The resultant N-hydroxy-4-(4-methoxyphenyl)sulfanyl-benzamidine pale gum was then used directly in the next preparation step without further purification.

### Step 3: Preparation of 3-[4-(4-methoxyphenyl)sulfanylphenyl]-5-(trifluoromethyl)-1,2,4-oxadiazole

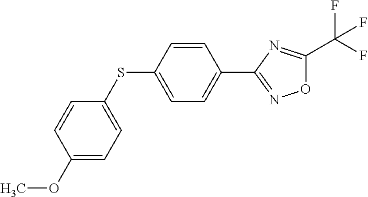 [SMILES: COc1ccc(Sc2ccc(-c3noc(C(F)(F)F)n3)cc2)cc1]

TFAA (1.5 equiv., 0.45 mL, 3.18 mmol) was introduced dropwise to a suspension of N-hydroxy-4-(4-methoxyphenyl)sulfanyl-benzamidine (0.58 g, 2.14 mmol) dissolved in THF (7.0 mL). The suspension was stirred for 3 hours until completion. After the solvent was removed at reduced pressure, the crude residue was purified by flash chromatography over silica gel (cyclohexane:EtOAc eluent gradient 1:0 to 3:1) to afford 0.55 g (74% yield) of 3-[4-(4-methoxyphenyl)sulfanylphenyl]-5-(trifluoromethyl)-1,2,4-oxadiazole as a yellow solid. LC/MS retention time=1.29 minutes, 353 (M+H); mp: 55-60° C.

¹H NMR (400 MHz, CDCl₃) δ ppm: 7.85 (m, 2H), 7.50 (m, 2H), 7.30 (d, 2H), 6.95 (d, 2H), 3.85 (s, 3H)

¹⁹F NMR (376 MHz, CDCl₃) δ ppm: −65.39 (s).

### Example 4: this Example Illustrates the Preparation of 3-[4-(4-methoxyphenyl)sulfinylphenyl]-5-(trifluoromethyl)-1,2,4-oxadiazole (Compound 2.2 of Table T2)

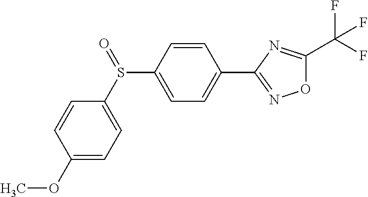 [SMILES: COc1ccc(S(=O)c2ccc(-c3noc(C(F)(F)F)n3)cc2)cc1]

To a solution of 3-(4-phenylsulfanylphenyl)-5-(trifluoromethyl)-1,2,4-oxadiazole (425 mg, 1.21 mmol) in dichloromethane (3.6 mL) was added m-chloroperoxybenzoic acid (1.1 equiv, 230 mg 1.33 mmol) at 25° C. After 2 hours, a saturated aqueous sodium bicarbonate solution was added, and the aqueous layer extracted with dichloromethane (2×30 mL) and the combined organic layers washed with brine, dried over sodium sulfate, and filtered. After the solvent was removed at reduced pressure, the crude residue was purified by flash chromatography over silica gel (cyclohexane:EtOAc eluent gradient 1:0 to 3:1) to afford 0.17 g (38% yield) of 3-[4-(4-methoxyphenyl)sulfinylphenyl]-5-(trifluoromethyl)-1,2,4-oxadiazole as a yellow solid. LC/MS retention time=1.03 minutes, 369 (M+H); mp: 93-98° C.

¹H NMR (400 MHz, CDCl₃) δ ppm: 8.22 (d, 2H), 7.76 (d, 2H), 7.60 (d, 2H), 6.98 (d, 2H), 3.85 (s, 3H).

¹⁹F NMR (376 MHz, CDCl₃) δ ppm: −65.33 (s).

### Example 5: this Example Illustrates the Preparation of 3-[4-(4-methoxyphenyl)sulfonylphenyl]-5-(trifluoromethyl)-1,2,4-oxadiazole (Compound 3.2 of Table T3)

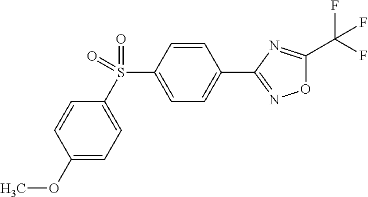 [SMILES: COc1ccc(S(=O)(=O)c2ccc(-c3noc(C(F)(F)F)n3)cc2)cc1]

To a solution of 3-(4-phenylsulfanylphenyl)-5-(trifluoromethyl)-1,2,4-oxadiazole (212 mg, 0.65 mmol) in dichloromethane (1.8 mL) was added m-chloroperoxybenzoic acid (1.6 equiv, 165 mg, 1.0 mmol) at 25° C. After reaction completion, a saturated aqueous solution of sodium bicarbonate was added, the aqueous layer extracted with dichloromethane (2×30 mL) and the combined organic layers washed with brine, dried over sodium sulfate, and filtered. After the solvent was removed at reduced pressure, the crude residue was purified by flash chromatography over silica gel (cyclohexane:EtOAc eluent gradient 1:0 to 3:1) to afford 0.11 g (51% yield) of 3-[4-(4-methoxyphenyl)sulfonylphenyl]-5-(trifluoromethyl)-1,2,4-oxadiazole as a yellow solid. LC/MS retention time=1.09 minutes, 385 (M+H); mp: 108-116° C.

¹H NMR (400 MHz, CDCl₃) δ ppm: 8.25 (d, 2H), 8.06 (d, 2H), 7.90 (d, 2H), 7.00 (d, 2H), 3.85 (s, 3H)

¹⁹F NMR (376 MHz, CDCl₃) δ ppm: −65.28 (s).

### Example 6: this Example Illustrates the Preparation of 3-(6-phenylsulfanyl-3-pyridyl)-5-(trifluoromethyl)-1,2,4-oxadiazole (Compound 1.7 of Table T1)

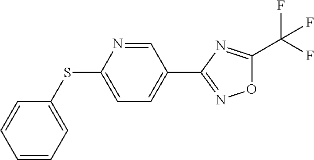 [SMILES: FC(F)(F)c1nc(-c2ccc(Sc3ccccc3)nc2)no1]

### Step 1: Preparation of 6-phenylsulfanylpyridine-3-carbonitrile

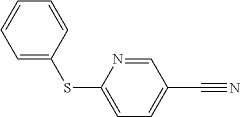 [SMILES: N#Cc1ccc(Sc2ccccc2)nc1]

To a dry flask charged with sodium hydride (1.2 equiv., 3.27 mmol, 60 mass % NaH) and DMF (3 mL), thiophenol (0.300 g, 2.72 mmol) was introduced dropwise over 30 min during which gas evolution was observed. 6-Chloropyridine-3-carbonitrile (1.1 equiv., 3.00 mmol) dissolved in DMF (1 mL) was introduced and the contents stirred for 1 hour at a temperature of 50° C. Upon reaction completion, the solution was quenched with water and extracted with ethyl acetate (2×50 mL). The organic layers were combined and dried over sodium sulfate and filtered. After the solvent was removed at reduced pressure, the crude residue was purified by flash chromatography over silica gel (cyclohexane: EtOAc eluent gradient 1:0 to 3:1) to afford 0.58 g (99% yield) of 6-phenylsulfanylpyridine-3-carbonitrile as a yellow solid. LC/MS retention time=1.09 minutes, 213 (M+H);

¹H NMR (400 MHz, CDCl₃) δ ppm: 8.64 (s, 1H), 7.65 (m, 3H), 7.50 (m, 3H), 6.90 (d, 1H).

### Step 2: Preparation of N-hydroxy-6-phenylsulfanyl-pyridine-3-carboxamidine

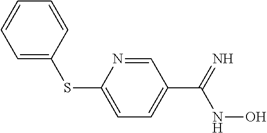 [SMILES: N=C(NO)c1ccc(Sc2ccccc2)nc1]

Hydroxylamine hydrochloride (3.0 equiv, 0.57 g, 8.24 mmol) was added at room temperature to a stirred solution of 6-phenylsulfanylpyridine-3-carbonitrile (0.58 g, 2.75 mmol) in ethanol (11 mL), followed by dropwise addition of triethylamine (3.0 equiv, 1.15 mL, 8.24 mmol). The resulting suspension was heated at a temperature of 80° C. for 60 min., cooled to 25° C., and concentrated under reduced pressure. The resultant N-hydroxy-6-phenylsulfanyl-pyridine-3-carboxamidine pale gum was then used directly in the next synthesis step without further purification.

### Step 3: Preparation of 3-(6-phenylsulfanyl-3-pyridyl)-5-(trifluoromethyl)-1,2,4-oxadiazole

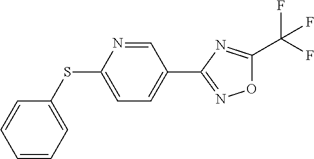 [SMILES: FC(F)(F)c1nc(-c2ccc(Sc3ccccc3)nc2)no1]

Trifluoroacetic anhydride (1.5 equiv., 0.58 mL, 4.12 mmol,) was introduced dropwise to a stirring solution of N-hydroxy-6-phenylsulfanyl-pyridine-3-carboxamidine (0.67 g, 2.75 mmol) dissolved in THF (9.0 mL). The suspension was stirred for 14 hours, achieving only 20% conversion, and a second addition of trifluoroacetic anhydride (1.5 equiv., 0.58 mL, 4.12 mmol,) with an additional 14 hours of stirring was needed for reaction completion. After the solvent was removed at reduced pressure, the crude residue was purified by flash chromatography over silica gel (cyclohexane:EtOAc eluent gradient 1:0 to 4:1) to afford 0.88 g (65% yield) of the 3-(6-phenylsulfanyl-3-pyridyl)-5-(trifluoromethyl)-1,2,4-oxadiazole as a yellow solid. LC/MS retention time=1.18 minutes, 324 (M+H); mp: 124-129° C.

¹H NMR (400 MHz, CDCl₃) δ ppm: 9.16 (s, 1H), 8.16 (d, 1H), 7.69 (m, 2H), 7.50 (m, 3H), 7.00 (d, 1H)

¹⁹F NMR (376 MHz, CDCl₃) δ ppm: −65.29 (s).

### Example 7: this Example Illustrates the Preparation of 3-[6-(benzenesulfinyl)-3-pyridyl]-5-(trifluoromethyl)-1,2,4-oxadiazole (Compound 2.10 of Table T2) and 3-[6-(benzenesulfonyl)-3-pyridyl]-5-(trifluoromethyl)-1,2,4-oxadiazole (Compound 3.7 of Table T3)

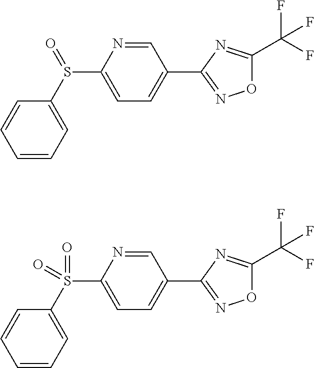 [SMILES: O=S(c1ccccc1)c1ccc(-c2noc(C(F)(F)F)n2)cn1 ; O=S(=O)(c1ccccc1)c1ccc(-c2noc(C(F)(F)F)n2)cn1]

To a solution of 3-(6-phenylsulfanyl-3-pyridyl)-5-(trifluoromethyl)-1,2,4-oxadiazole (541 mg, 1.21 mmol) in dichloromethane (5.0 mL) was added m-chloroperoxybenzoic acid (1.1 equiv, 318 mg 1.84 mmol) at 25° C. After 60 minutes, a saturated aqueous solution of sodium bicarbonate was added and the aqueous layer extracted with dichloromethane (2×30 mL). The combined organic layers were washed with brine, dried over sodium sulfate, and then filtered. After the solvent was removed at reduced pressure, the crude residue was purified by flash chromatography over silica gel (cyclohexane:EtOAc eluent gradient 1:0 to 10:1). A first chromatography fraction afforded 0.30 g (52% yield) of compound 2.10 of Table T2 (3-[6-(benzenesulfinyl)-3-pyridyl]-5-(trifluoromethyl)-1,2,4-oxadiazole) as a yellow solid [LC/MS retention time=1.03 minutes, 369 (M+H); mp: 91-97° C.] and a second chromatography fraction provided compound 3.7 of Table T3 (3-[6-(benzenesulfonyl)-3-pyridyl]-5-(trifluoromethyl)-1,2,4-oxadiazole) as a yellow solid (0.15 g, 25% yield) [LC/MS retention time=1.01 minutes, 356 (M+H); mp: 124-127° C.].

[Compound 2.10 of Table T2]

¹H NMR (400 MHz, CDCl₃) δ ppm: 9.27 (s, 1H), 8.57 (dd, 1H), 8.15 (dd, 1H), 7.84 (m, 2H), 7.50 (m, 3H).

¹⁹F NMR (376 MHz, CDCl₃) δ ppm: −65.25 (s).

[Compound 3.7 of Table T3]

¹H NMR (400 MHz, CDCl₃) δ ppm: 9.40 (s, 1H), 8.67 (dd, 1H), 8.41 (dd, 1H), 8.17 (dd, 2H), 7.71 (m, 1H), 7.61 (m, 2H).

¹⁹F NMR (376 MHz, CDCl₃) δ ppm: −65.17 (s).

### Example 8: this Example Illustrates the Preparation 3-[2,3-difluoro-4-(phenylsulfanylmethyl)phenyl]-5-(trifluoromethyl)-1,2,4-oxadiazole (Compound 1.26 of Table T1)

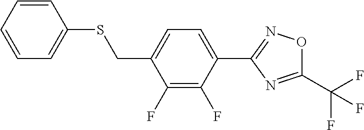 [SMILES: Fc1c(CSc2ccccc2)ccc(-c2noc(C(F)(F)F)n2)c1F]

### Step 1: Preparation of 2,3-difluoro-N′-hydroxy-4-methyl-benzamidine

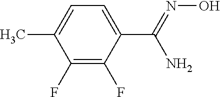

To a suspension of 2,3-difluoro-4-methylbenzonitrile (5.0 g, 32.6 mmol) in ethanol (111 mL) at 25° C. was added hydroxylamine hydrochloride (4.5 g, 65.3 mmol). The reaction mixture was heated at 80° C. for 2 h and after cooling to room temperature the volatiles were removed under reduced pressure to give 2,3-difluoro-N′-hydroxy-4-methyl-benzamidine as a white solid that was used in the next step without purification.

¹H NMR (400 MHz, CDCl₃) δ ppm: 7.30 (m, 1H), 6.95 (m, 1H), 6.50 (brs, 1H), 5.05 (brs, 2H), 2.30 (s, 3H).

### Step 2: Preparation of 3-(2,3-difluoro-4-methyl-phenyl)-5-(trifluoromethyl)-1,2,4-oxadiazole

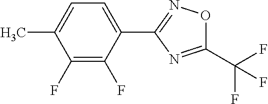 [SMILES: Cc1ccc(-c2noc(C(F)(F)F)n2)c(F)c1F]

To a solution of 2,3-difluoro-N′-hydroxy-4-methyl-benzamidine (2.6 mmol) in tetrahydrofuran (108 mL) cooled using an ice bath was added TFAA (6.9 mL, 49 mmol). The reaction mixture was stirred at 25° C. overnight and then diluted with water. The organic layer was separated, washed successively with a saturated aqueous sodium bicarbonate solution, a saturated aqueous ammonium chloride solution and water, and then dried over sodium sulfate, filtered, and evaporated to dryness. The crude 3-(2,3-difluoro-4-methyl-phenyl)-5-(trifluoromethyl)-1,2,4-oxadiazole (6.6 g, 72% yield) was isolated as a light brown solid that was used in the next transformation without further purification. LC/MS (Method A) retention time=1.16 minutes, 265 (M+H).

¹H NMR (400 MHz, CDCl₃) δ ppm: 7.76 (d, 1H), 7.12 (d, 1H), 2.41 (s, 3H).

¹⁹F NMR (400 MHz, CDCl₃) δ ppm: −65.41 (s), −133.3 (s), −140.1 (s).

### Step 3: Preparation of 3-[4-(bromomethyl)-2,3-difluoro-phenyl]-5-(trifluoromethyl)-1,2,4-oxadiazole

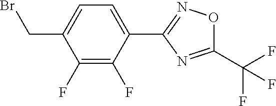 [SMILES: Fc1c(CBr)ccc(-c2noc(C(F)(F)F)n2)c1F]

A mixture of 3-(2,3-difluoro-4-methyl-phenyl)-5-(trifluoromethyl)-1,2,4-oxadiazole (6.0 g, 22.6 mmol) and NBS (7.17 g, 10.0 mmol) in tetrachloromethane (79 mL) under argon was heated to 70° C. AIBN (0.68 g, 3.95 mmol) was added and the reaction mixture stirred at 65° C. for 36 h. The mixture was cooled to 25° C., diluted with dichloromethane and water, and the layers were separated. The succinimide by-product was filtered off, and the solvent was removed under vacuum to afford a brown gum. This crude residue was subjected to flash chromatography over silica gel (cyclohexane:EtOAc eluent gradient 100:0 to 4:1) to afford 3-[4-(bromomethyl)-2,3-difluoro-phenyl]-5-(trifluoromethyl)-1,2,4-oxadiazole as a white solid (4.8 g, 72% yield). LC/MS (Method A) retention time=1.16 minutes, 344 (M+H).

¹H NMR (400 MHz, CDCl₃) δ ppm: 7.80 (m, 1H), 7.37 (m, 1H), 4.55 (s, 2H).

¹⁹F NMR (400 MHz, CDCl₃) δ ppm: −65.1 (s), −131.2 (s), −139.1 (s).

### Step 4: Preparation of 3-[2,3-difluoro-4-(phenylsulfanylmethyl)phenyl]-5-(trifluoromethyl)-1,2,4-oxadiazole

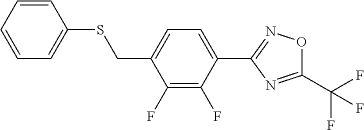 [SMILES: Fc1c(CSc2ccccc2)ccc(-c2noc(C(F)(F)F)n2)c1F]

To a solution of 3-[4-(bromomethyl)-2,3-difluoro-phenyl]-5-(trifluoromethyl)-1,2,4-oxadiazole (310 mg, 0.90 mmol) in dichloromethane (2 mL) at 25° C. was added thiophenol (1.1 equiv, 0.84 mL, 1.0 mmol) and potassium carbonate (1.0 equiv, 0.13 g, 0.90 mmol). The reaction mixture was stirred for 12 hours at 40° C. then poured onto water and the layers were separated. The aqueous layer was extracted with ethyl acetate (2×30 mL) and the combined organic layers were washed with brine, dried over sodium sulfate, and filtered. The solvent was removed under reduced pressure and the resultant crude residue was purified by flash chromatography over silica gel (cyclohexane:EtOAc eluent gradient 1:0 to 1:1) to give 0.320 g (97% yield) of 3-[2,3-difluoro-4-(phenylsulfanylmethyl)phenyl]-5-(trifluoromethyl)-1,2,4-oxadiazole as a yellow oil which solidified to a solid upon standing. LC/MS retention time=1.31 minutes, 389 (M+17), mp: 52-58° C.

¹H NMR (400 MHz, CDCl₃) δ ppm: 7.72 (m, 1H), 7.29 (m, 2H), 7.25 (m, 3H), 7.11 (m, 1H), 4.16 (s, 2H).

¹⁹F NMR (400 MHz, CDCl₃) δ ppm: −65.16 (s), −132.1 (s), −140.1 (s).

### Example 9: this Example Illustrates the Preparation of 3-[4-(benzenesulfinylmethyl)-2,3-difluoro-phenyl]-5-(trifluoromethyl)-1,2,4-oxadiazole (Compound 2.31 of Table T2) and 3-[4-(benzenesulfonylmethyl)-2,3-difluoro-phenyl]-5-(trifluoromethyl)-1,2,4-oxadiazole (Compound 3.23 of Table T3)

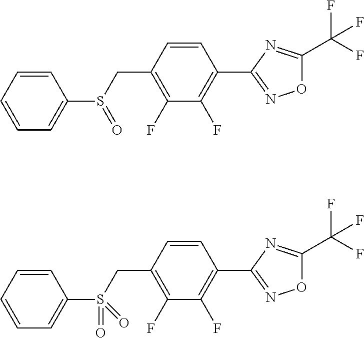 [SMILES: O=S(Cc1ccc(-c2noc(C(F)(F)F)n2)c(F)c1F)c1ccccc1 ; O=S(=O)(Cc1ccc(-c2noc(C(F)(F)F)n2)c(F)c1F)c1ccccc1]

To a solution of 3-[2,3-difluoro-4-(phenylsulfanylmethyl)phenyl]-5-(trifluoromethyl)-1,2,4-oxadiazole (306 mg, 0.82 mmol) in dichloromethane (10 mL) was added m-chloroperoxybenzoic acid (1.1 equiv, 159 mg, 0.9 mmol) at 25° C. After 60 minutes, a second portion of m-chloroperoxybenzoic acid (0.5 equiv, 80 mg, 0.45 mmol) was introduced. After 30 minutes, a saturated aqueous solution of sodium bicarbonate was added and the aqueous layer was extracted with dichloromethane (2×30 mL). The combined organic layers were washed with brine, dried over sodium sulfate, and then filtered. After the solvent was removed at reduced pressure the crude residue is purified by flash chromatography over silica gel (cyclohexane:EtOAc eluent gradient 1:0 to 10:1). A first chromatography fraction afforded 0.167 g (58% yield) of compound 2.31 of Table T2 (3-[4-(benzenesulfinylmethyl)-2,3-difluoro-phenyl]-5-(trifluoromethyl)-1,2,4-oxadiazole) as a yellow solid [LC/MS retention time=1.06 minutes, 389 (M+1); mp: 163-167° C.] and a second chromatography fraction provided compound 3.23 of Table T3 (3-[4-(benzenesulfonylmethyl)-2,3-difluoro-phenyl]-5-(trifluoromethyl)-1,2,4-oxadiazole) as a yellow solid (0.10 g, 27% yield) [LC/MS retention time=1.08 minutes, 403 (M-1); mp: 187-191° C.].

[Compound 2.31 of Table T2]

¹H NMR (400 MHz, CDCl₃) δ ppm: 7.81 (m, 1H), 7.50 (m, 5H), 7.08 (m, 1H), 4.18 (m, 2H).

¹⁹F NMR (376 MHz, CDCl₃) δ ppm: −65.25 (s), −131.8 (s), −138.9 (s).

[Compound 3.23 of Table T3]

¹H NMR (400 MHz, CDCl₃) δ ppm: 7.87 (s, 1H), 7.74 (m, 2H), 7.68 (m, 1H), 7.54 (m, 2H), 7.30 (m, 1H), 7.49 (m, 2H).

¹⁹F NMR (376 MHz, CDCl₃) δ ppm: −65.12 (s), −130.8 (s), −138.5 (s).

### Example 10: this Example Illustrates the Preparation 3-[6-(phenylsulfanylmethyl)-3-pyridyl]-5-(trifluoromethyl)-1,2,4-oxadiazole (Compound 1.23 of Table T1)

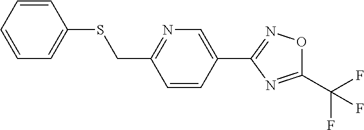 [SMILES: FC(F)(F)c1nc(-c2ccc(CSc3ccccc3)nc2)no1]

### Step 1: Preparation of N′-hydroxy-6-methyl-pyridine-3-carboxamidine

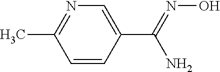

To a suspension of 5-cyano-2-picoline (3 g, 25.0 mmol) in ethanol (86 mL) at 25° C. was added hydroxylamine hydrochloride (5.3 g, 76 mmol). The reaction mixture was heated at 80° C. for 2 h. After cooling to room temperature, the volatiles were removed under reduced pressure to afford N′-hydroxy-6-methyl-pyridine-3-carboxamidine as a white solid that was used in the next step without any purification. LC/MS (Method A) retention time=0.17 minutes, 152 (M+H).

¹H NMR (400 MHz, CDCl₃) δ ppm: 8.75 (s, 1H), 7.83 (d, 1H), 7.19 (d, 1H), 4.86 (brs, 2H), 2.63 (s, 3H).

### Step 2: Preparation of 3-(6-methyl-3-pyridyl)-5-(trifluoromethyl)-1,2,4-oxadiazole

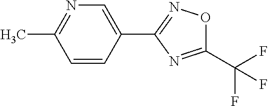 [SMILES: Cc1ccc(-c2noc(C(F)(F)F)n2)cn1]

To a solution of N′-hydroxy-6-methyl-pyridine-3-carboxamidine (25 mmol) in tetrahydrofuran (84 mL) cooled via an ice bath was added TFAA (5.28 mL, 38.0 mmol). The reaction mixture was stirred at 25° C. overnight and then diluted with water. The organic layer was separated, washed successively with sodium bicarbonate solution, ammonium chloride solution and water, and then dried over sodium sulfate, filtered and evaporated to dryness to afford 3-(6-methyl-3-pyridyl)-5-(trifluoromethyl)-1,2,4-oxadiazole (5.8 g, 84% yield) as an amorphous white solid. LC/MS (Method A) retention time=1.14 minutes, 247 (M+H).

¹H NMR (400 MHz, CDCl₃) δ ppm: 9.23 (d, 1H), 8.27 (dd, 1H), 7.33 (d, 1H), 2.63 (s, 3H).

¹⁹F NMR (400 MHz, CDCl₃) δ ppm: −65.3 (s).

### Step 3: Preparation of 3-[6-(bromomethyl)-3-pyridyl]-5-(trifluoromethyl)-1,2,4-oxadiazole

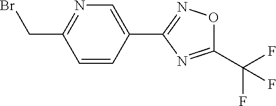 [SMILES: FC(F)(F)c1nc(-c2ccc(CBr)nc2)no1]

A solution of 3-(6-methyl-3-pyridyl)-5-(trifluoromethyl)-1,2,4-oxadiazole (4.4 g, 19 mmol), AIBN (0.32 g, 1.9 mmol), and tetrachloromethane (38 mL) under argon was heated to 65° C. NBS (3.11 g, 17.1 mmol) was added portion-wise and the reaction mixture stirred at 65° C. for 5 h and then a second equivalent of NBS (3.11 g, 17.1 mmol) added and stirring continued overnight. The mixture was cooled to 25° C. then diluted with dichloromethane and water after which the layers were separated. The succinimide by-product was filtrated off, and the solvent was removed under reduced pressure to afford a brown gum. This crude residue was subjected to flash chromatography over silica gel (cyclohexane:EtOAc eluent gradient 100:0 to 4:1) to afford 3-[6-(bromomethyl)-3-pyridyl]-5-(trifluoromethyl)-1,2,4-oxadiazole as a white solid (5.9 g, 37% yield. LC/MS (Method A) retention time=1.01 minutes, 308 (M+H).

¹H NMR (400 MHz, CDCl₃) δ ppm: 9.30 (d, 1H), 8.40 (dd, 1H), 7.63 (d, 1H), 4.62 (s, 2H).

¹⁹F NMR (400 MHz, CDCl₃) δ ppm: −65.2 (s).

### Step 4: Preparation of 3-[6-(phenylsulfanylmethyl)-3-pyridyl]-5-(trifluoromethyl)-1,2,4-oxadiazole

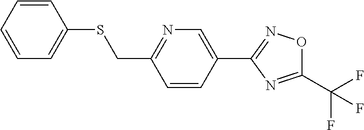 [SMILES: FC(F)(F)c1nc(-c2ccc(CSc3ccccc3)nc2)no1]

To a solution of 3-[4-(bromomethyl)-2,3-difluoro-phenyl]-5-(trifluoromethyl)-1,2,4-oxadiazole (174 mg, 0.90 mmol) in dichloromethane (2 mL) at 25° C. was added thiophenol (1.1 equiv, 0.5 mL, 0.62 mmol) and potassium carbonate (1.0 equiv, 78 mg, 0.57 mmol). The reaction mixture was stirred overnight at 40° C. then poured onto water and the layers were separated. The aqueous layer was extracted with ethyl acetate (2×30 mL) and the combined organic layers washed with brine, dried over sodium sulfate, and filtered. The solvent was removed under reduced pressure and the resultant crude residue was purified by flash chromatography over silica gel (cyclohexane:EtOAc eluent gradient 1:0 to 1:1) to give 0.15 g (85% yield) of 3-[6-(phenylsulfanylmethyl)-3-pyridyl]-5-(trifluoromethyl)-1,2,4-oxadiazole as a yellow oil which solidified to a solid upon standing. LC/MS retention time=1.19 minutes, 338 (M+1), mp: 71-78° C.

¹H NMR (400 MHz, CDCl₃) δ ppm: 9.23 (d, 1H), 8.22 (d, 1H), 7.40 (d, 2H), 7.25 (m, 1H), 7.13 (m, 3H), 4.25 (s, 2H).

¹⁹F NMR (400 MHz, CDCl₃) δ ppm: −65.24 (s)

### Example 11: this Example Illustrates the Preparation of 3-[6-(benzenesulfinylmethyl)-3-pyridyl]-5-(trifluoromethyl)-1,2,4-oxadiazole (Compound 2.28 of Table T2) and 3-[6-(benzenesulfonylmethyl)-3-pyridyl]-5-(trifluoromethyl)-1,2,4-oxadiazole (Compound 3.21 of Table T3)

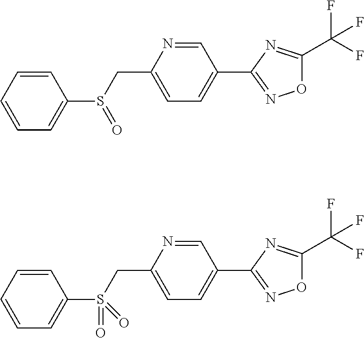 [SMILES: O=S(Cc1ccc(-c2noc(C(F)(F)F)n2)cn1)c1ccccc1 ; O=S(=O)(Cc1ccc(-c2noc(C(F)(F)F)n2)cn1)c1ccccc1]

To a solution of 3-[6-(phenylsulfanylmethyl)-3-pyridyl]-5-(trifluoromethyl)-1,2,4-oxadiazole (134 mg, 0.40 mmol) in dichloromethane (6 mL) was added m-chloroperoxybenzoic acid (1.1 equiv, 75 mg, 0.44 mmol) at 25° C. After 60 minutes, a second portion of m-chloroperoxybenzoic acid (0.5 equiv, 38 mg, 0.42 mmol) was introduced. After 30 minutes, a saturated aqueous solution of sodium bicarbonate was added and the aqueous layer extracted with dichloromethane (2×30 mL). The combined organic layers were washed with brine, dried over sodium sulfate, and then filtered. After the solvent was removed at reduced pressure, the crude residue was purified by flash chromatography over silica gel (cyclohexane:EtOAc eluent gradient 1:0 to 10:1). A first chromatography fraction afforded 0.05 g (36% yield) of compound 2.28 of Table T2 (3-[6-(benzenesulfinylmethyl)-3-pyridyl]-5-(trifluoromethyl)-1,2,4-oxadiazole) as a yellow solid [LC/MS retention time=0.93 minutes, 354 (M+1); mp: 116-120° C.] and a second chromatography fraction provided compound 3.21 of Table T3 (3-[6-(benzenesulfonylmethyl)-3-pyridyl]-5-(trifluoromethyl)-1,2,4-oxadiazole) as a yellow solid (0.05 g, 33% yield) [LC/MS retention time=0.98 minutes, 370 (M-1); mp: 160-166° C.].

[Compound 2.28 of Table T2]

¹H NMR (400 MHz, CDCl₃) δ ppm: 9.22 (s, 1H), 8.34 (dd, 1H), 7.52 (m, 5H), 7.38 (d, 1H), 4.29 (m, 2H).

¹⁹F NMR (376 MHz, CDCl₃) δ ppm: −65.23 (s).

[Compound 3.21 of Table T3]

¹H NMR (400 MHz, CDCl₃) δ ppm: 9.12 (s, 1H), 8.41 (dd, 1H), 7.71 (m, 2H), 7.62 (m, 2H), 7.49 (m, 2H), 4.65 (m, 2H).

¹⁹F NMR (376 MHz, CDCl₃) δ ppm: −65.12 (s).

Where necessary, enantiomerically pure final compounds may be obtained from racemic materials as appropriate via standard physical separation techniques, such as reverse phase chiral chromatography, or through stereoselective synthetic techniques, (eg, by using chiral starting materials).

TABLE T1
Melting point (mp) data and/or retention times (RT) for the compounds of Formula (I).

| | | | RT | [M + Z] | Mp |
|---|---|---|---|---|---|
| Entry | Compound name | Structure | (min) | (measured) | (° C.) |
| 1.1 | 3-(4- phenylsulfanylphenyl)-5- (trifluoromethyl)-1,2,4- oxadiazole | 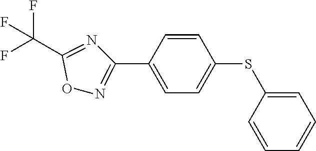 [SMILES: FC(F)(F)c1nc(-c2ccc(Sc3ccccc3)cc2)no1] | 1.29 | 323 (M + 1) | 59-62 |
| 1.2 | 3-[4-(4- methoxyphenyl)sulfanyl- phenyl]-5-(trifluoromethyl)- 1,2,4-oxadiazole | 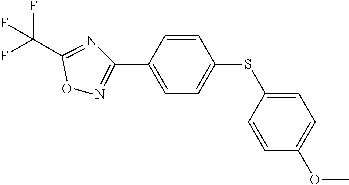 [SMILES: COc1ccc(Sc2ccc(-c3noc(C(F)(F)F)n3)cc2)cc1] | 1.29 | 370 (M + 18) | 55-60 |
| 1.3 | 3-(4- benzylsulfanylphenyl)-5- (trifluoromethyl)-1,2,4- oxadiazole | 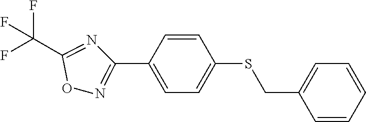 [SMILES: FC(F)(F)c1nc(-c2ccc(SCc3ccccc3)cc2)no1] | 1.27 | 337 (M + H) | 83-87 |
| 1.4 | 3-[4-(2,2,2- trifluoroethylsulfanyl)phenyl]- 5-(trifluoromethyl)- 1,2,4- oxadiazole | 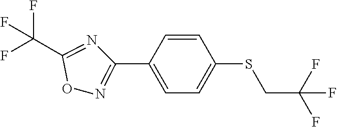 [SMILES: FC(F)(F)CSc1ccc(-c2noc(C(F)(F)F)n2)cc1] | 1.18 | 346 (M + 18) | |
| 1.5 | 3-[4-(2- phenylethylsulfanyl)phenyl]- 5-(trifluoromethyl)-1,2,4- oxadiazole | 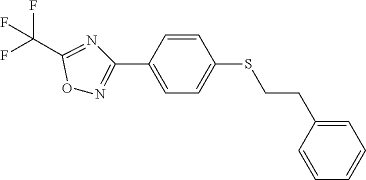 [SMILES: FC(F)(F)c1nc(-c2ccc(SCCc3ccccc3)cc2)no1] | 1.3 | 368 (M + 18) | |
| 1.6 | 3-[4-[(2,6- difluorophenyl)methylsulfanyl] phenyl]-5- (trifluoromethyl)-1,2,4- oxadiazole | 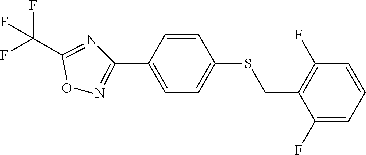 [SMILES: Fc1cccc(F)c1CSc1ccc(-c2noc(C(F)(F)F)n2)cc1] | 1.27 | 390 (M + 18) | |
| 1.7 | 3-(6-phenylsulfanyl-3- pyridyl)-5-(trifluoromethyl)- 1,2,4-oxadiazole | 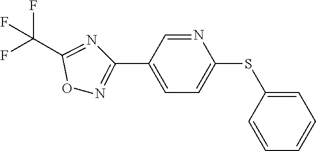 [SMILES: FC(F)(F)c1nc(-c2ccc(Sc3ccccc3)nc2)no1] | 1.18 | 324 (M + 1) | |
| 1.8 | 3-(3-methyl-4- phenylsulfanyl-phenyl)-5- (trifluoromethyl)-1,2,4- oxadiazole | 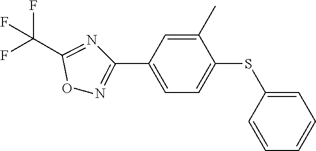 [SMILES: Cc1cc(-c2noc(C(F)(F)F)n2)ccc1Sc1ccccc1] | 1.34 | 354 (M + 18) | |
| 1.9 | 3-[4-(4- fluorophenyl)sulfanylphenyl]- 5-(trifluoromethyl)-1,2,4- oxadiazole | 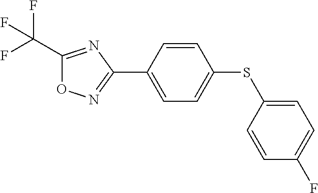 [SMILES: Fc1ccc(Sc2ccc(-c3noc(C(F)(F)F)n3)cc2)cc1] | 1.29 | 358 (M + 18) | 64-67 |
| 1.10 | 3-[4-(2,4- difluorophenyl)sulfanylphenyl]- 5-(trifluoromethyl)- 1,2,4-oxadiazole | 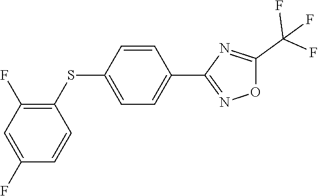 [SMILES: Fc1ccc(Sc2ccc(-c3noc(C(F)(F)F)n3)cc2)c(F)c1] | 1.27 | 378 (M + 18) | 72-80 |
| 1.11 | 3-[4-(2- methoxyphenyl)sulfanyl- phenyl]-5-(trifluoromethyl)- 1,2,4-oxadiazole | 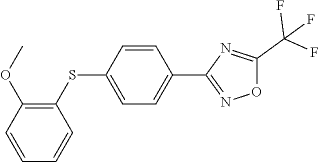 [SMILES: COc1ccccc1Sc1ccc(-c2noc(C(F)(F)F)n2)cc1] | 1.3 | 370 (M + 18) | |
| 1.12 | 3-[4-(2,6- difluorophenyl)sulfanylphenyl]- 5-(trifluoromethyl)- 1,2,4-oxadiazole | 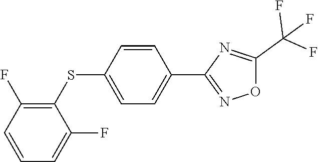 [SMILES: Fc1cccc(F)c1Sc1ccc(-c2noc(C(F)(F)F)n2)cc1] | 1.3 | 375 (M + 18) | 75-85 |
| 1.13 | 3-[4- (phenylsulfanylmethyl) phenyl]-5-(trifluoromethyl)- 1,2,4-oxadiazole | 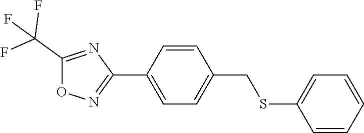 [SMILES: FC(F)(F)c1nc(-c2ccc(CSc3ccccc3)cc2)no1] | 1.3 | 336 (M + 1) | 86-91 |
| 1.14 | 3-[4-(2- fluorophenyl)sulfanylphenyl]- 5-(trifluoromethyl)-1,2,4- oxadiazole | 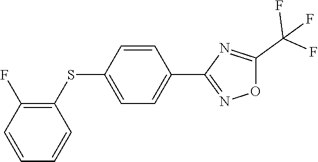 [SMILES: Fc1ccccc1Sc1ccc(-c2noc(C(F)(F)F)n2)cc1] | 1.31 | 358 (M + 18) | 35-38 |
| 1.15 | 3-[4-[(2- fluorophenyl)sulfanylmethyl] phenyl]-5- (trifluoromethyl)-1,2,4- oxadiazole | 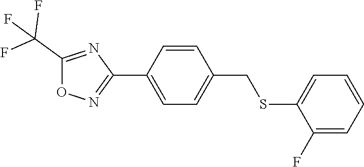 [SMILES: Fc1ccccc1SCc1ccc(-c2noc(C(F)(F)F)n2)cc1] | 1.3 | 354 (M + 1) | 56-61 |
| 1.16 | 3-(4-tert- butylsulfanylphenyl)-5- (trifluoromethyl)-1,2,4- oxadiazole | 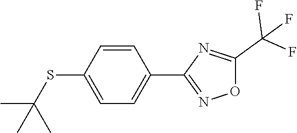 [SMILES: CC(C)(C)Sc1ccc(-c2noc(C(F)(F)F)n2)cc1] | 1.35 | 320 (M + 18) | |
| 1.17 | 3-[4-(o- tolylsulfanyl)phenyl]-5- (trifluoromethyl)-1,2,4- oxadiazole | 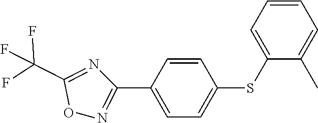 [SMILES: Cc1ccccc1Sc1ccc(-c2noc(C(F)(F)F)n2)cc1] | 1.34 | 354 (M + 18) | |
| 1.18 | 3-[4-(2- chlorophenyl)sulfanylphenyl]- 5-(trifluoromethyl)- 1,2,4-oxadiazole | 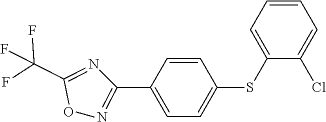 [SMILES: FC(F)(F)c1nc(-c2ccc(Sc3ccccc3Cl)cc2)no1] | 1.31 | 338 (M + 18) | |
| 1.19 | 3-[6-(o-tolylsulfanyl)-3- pyridyl]-5-(trifluoromethyl)- 1,2,4-oxadiazole | 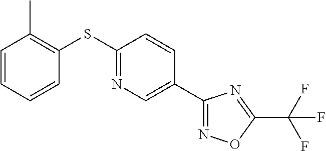 | 1.35 | 334 (M + 18) | |
| 1.20 | 3-[6-(2- fluorophenyl)sulfanyl-3- pyridyl]-5-(trifluoromethyl)- 1,2,4-oxadiazole | 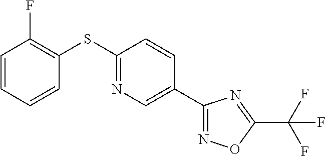 [SMILES: Fc1ccccc1Sc1ccc(-c2noc(C(F)(F)F)n2)cn1] | 1.22 | 342 (M + 1) | |
| 1.21 | 3-(6-benzylsulfanyl-3- pyridyl)-5-(trifluoromethyl)- 1,2,4-oxadiazole | 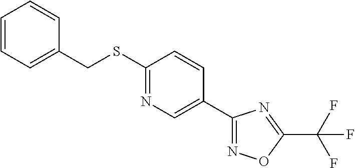 [SMILES: FC(F)(F)c1nc(-c2ccc(SCc3ccccc3)nc2)no1] | 1.29 | 338 (M + 1) | 66-68 |
| 1.22 | 3-(4-benzylsulfanyl-3- methyl-phenyl)-5- (trifluoromethyl)-1,2,4- oxadiazole | 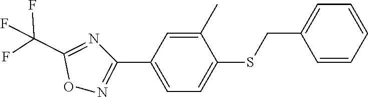 [SMILES: Cc1cc(-c2noc(C(F)(F)F)n2)ccc1SCc1ccccc1] | 1.31 | Mass not detected | 66-72 |
| 1.23 | 3-[6- (phenylsulfanylmethyl)-3- pyridyl]-5-(trifluoromethyl)- 1,2,4-oxadiazole | 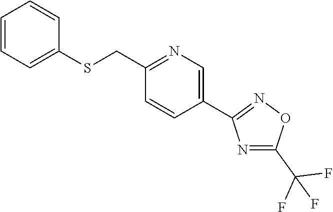 [SMILES: FC(F)(F)c1nc(-c2ccc(CSc3ccccc3)nc2)no1] | 1.19 | 338 (M + 1) | 71-78 |
| 1.24 | 3-[3-fluoro-4- (phenylsulfanylmethyl) phenyl]-5-(trifluoromethyl)- 1,2,4-oxadiazole | 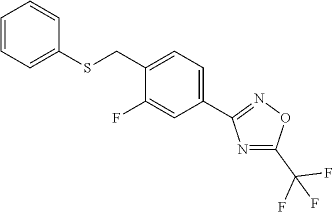 [SMILES: Fc1cc(-c2noc(C(F)(F)F)n2)ccc1CSc1ccccc1] | 1.29 | 354 (M + 17) | 50-55 |
| 1.25 | 3-[2-fluoro-4- (phenylsulfanylmethyl) phenyl]-5-(trifluoromethyl)- 1,2,4-oxadiazole | 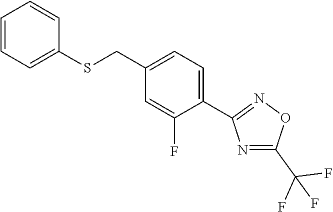 [SMILES: Fc1cc(CSc2ccccc2)ccc1-c1noc(C(F)(F)F)n1] | 1.32 | 353 (M − 1) | 48-54 |
| 1.26 | 3-[2,3-difluoro-4- (phenylsulfanylmethyl) phenyl]-5-(trifluoromethyl)- 1,2,4-oxadiazole | 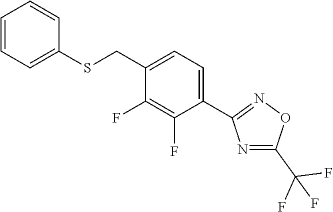 [SMILES: Fc1c(CSc2ccccc2)ccc(-c2noc(C(F)(F)F)n2)c1F] | 1.31 | 389 (M + 17) | 52-58 |
| 1.27 | 3-[3,5-difluoro-4- (phenylsulfanylmethyl) phenyl]-5-(trifluoromethyl)- 1,2,4-oxadiazole | 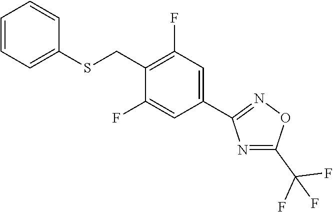 [SMILES: Fc1cc(-c2noc(C(F)(F)F)n2)cc(F)c1CSc1ccccc1] | 1.31 | 389 (M + 17) | 50-55 |

TABLE T2
Melting point (mp) data and/or retention times (RT) for the compounds of Formula (I).

| | | | RT | [M + H] | Mp |
|---|---|---|---|---|---|
| Entry | Compound name | Structure | (min) | (measured) | (° C.) |
| 2.1 | 3-[4- (benzenesulfinyl)phenyl]- 5-(trifluoromethyl)-1,2,4- oxadiazole | 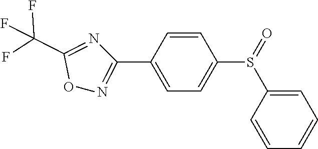 [SMILES: O=S(c1ccccc1)c1ccc(-c2noc(C(F)(F)F)n2)cc1] | 1.0 | 339 | 97- 106 |
| 2.2 | 3-[4-(4- methoxyphenyl)sulfinyl- phenyl]-5-(trifluoromethyl)- 1,2,4-oxadiazole | 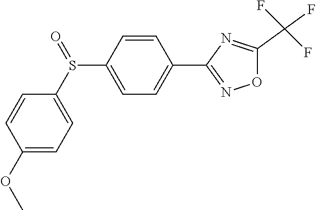 [SMILES: COc1ccc(S(=O)c2ccc(-c3noc(C(F)(F)F)n3)cc2)cc1] | 1.03 | 369 | 93-98 |
| 2.3 | 3-(4- cyclohexylsulfinylphenyl)- 5-(trifluoromethyl)-1,2,4- oxadiazole | 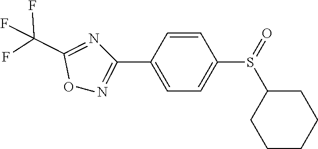 [SMILES: O=S(c1ccc(-c2noc(C(F)(F)F)n2)cc1)C1CCCCC1] | 1.09 | 345 | 108- 114 |
| 2.4 | 3-(4-benzylsulfinylphenyl)- 5-(trifluoromethyl)-1,2,4- oxadiazole | 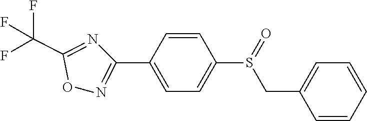 [SMILES: O=S(Cc1ccccc1)c1ccc(-c2noc(C(F)(F)F)n2)cc1] | 1.04 | 353 | 178- 181 |
| 2.5 | 3-[4-(2,4- difluorophenyl)sulfinyl- phenyl]-5-(trifluoromethyl)- 1,2,4-oxadiazole | 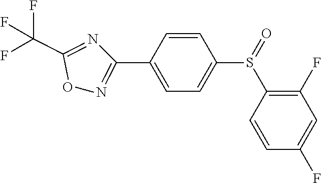 [SMILES: O=S(c1ccc(-c2noc(C(F)(F)F)n2)cc1)c1ccc(F)cc1F] | 1.09 | 375 | 118- 123 |
| 2.6 | 3-[4-(2,2,2- trifluoroethylsulfinyl) phenyl]-5-(trifluoromethyl)- 1,2,4- oxadiazole | 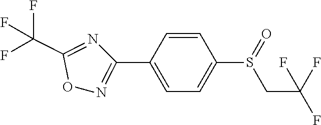 [SMILES: O=S(CC(F)(F)F)c1ccc(-c2noc(C(F)(F)F)n2)cc1] | 0.99 | 345 | 93- 100 |
| 2.7 | 3-[4-(2,5- dimethylphenyl)sulfinyl- phenyl]-5-(trifluoromethyl)- 1,2,4-oxadiazole | 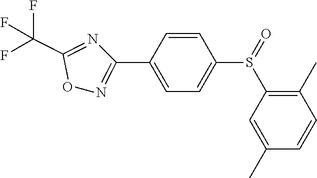 [SMILES: Cc1ccc(C)c(S(=O)c2ccc(-c3noc(C(F)(F)F)n3)cc2)c1] | 1.12 | 367 | 103- 109 |
| 2.8 | 3-[4-(2- phenylethylsulfinyl)phenyl]- 5-(trifluoromethyl)-1,2,4- oxadiazole | 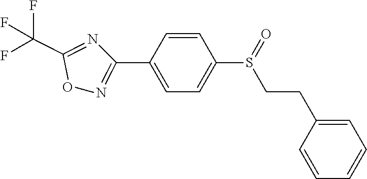 [SMILES: O=S(CCc1ccccc1)c1ccc(-c2noc(C(F)(F)F)n2)cc1] | 1.07 | 367 | 81-85 |
| 2.9 | 3-[4-[(2,6- difluorophenyl)methyl- sulfinyl]phenyl]-5- (trifluoromethyl)-1,2,4- oxadiazole | 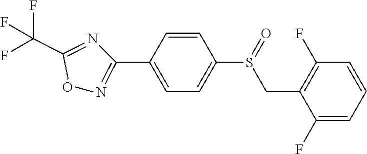 [SMILES: O=S(Cc1c(F)cccc1F)c1ccc(-c2noc(C(F)(F)F)n2)cc1] | 1.05 | 389 | 126- 130 |
| 2.10 | 3-[6-(benzenesulfinyl)-3- pyridyl]-5-(trifluoromethyl)- 1,2,4-oxadiazole | 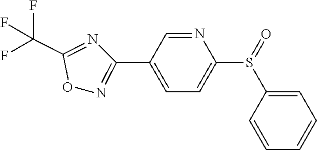 [SMILES: O=S(c1ccccc1)c1ccc(-c2noc(C(F)(F)F)n2)cn1] | 1.00 | 340 | 91-97 |
| 2.11 | 3-[4-(benzenesulfinyl)-3- methyl-phenyl]-5- (trifluoromethyl)-1,2,4- oxadiazole | 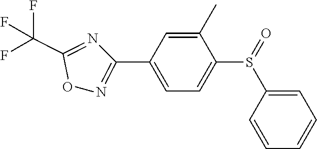 [SMILES: Cc1cc(-c2noc(C(F)(F)F)n2)ccc1S(=O)c1ccccc1] | 1.07 | 353 | |
| 2.12 | 3-[4-(3,5- dimethylphenyl)sulfinyl- phenyl]-5-(trifluoromethyl)- 1,2,4-oxadiazole | 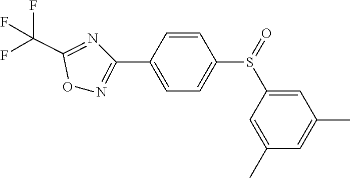 [SMILES: Cc1cc(C)cc(S(=O)c2ccc(-c3noc(C(F)(F)F)n3)cc2)c1] | 1.14 | 367 | 71-83 |
| 2.13 | 3-[4-(4- fluorophenyl)sulfinylphenyl]- 5-(trifluoromethyl)-1,2,4- oxadiazole | 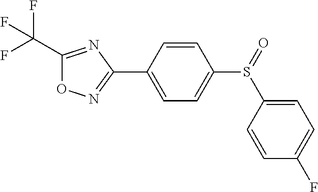 [SMILES: O=S(c1ccc(F)cc1)c1ccc(-c2noc(C(F)(F)F)n2)cc1] | 1.04 | 357 | 94-99 |
| 2.14 | 5-(trifluoromethyl)-3-[4-[4- (trifluoromethyl)phenyl] sulfinylphenyl]-1,2,4- oxadiazole | 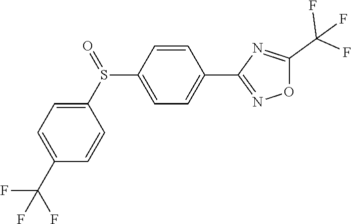 [SMILES: O=S(c1ccc(-c2noc(C(F)(F)F)n2)cc1)c1ccc(C(F)(F)F)cc1] | 1.12 | 407 | 94- 100 |
| 2.15 | 3-[4-(2- methoxyphenyl)sulfinyl- phenyl]-5-(trifluoromethyl)- 1,2,4-oxadiazole | 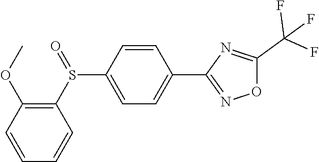 [SMILES: COc1ccccc1S(=O)c1ccc(-c2noc(C(F)(F)F)n2)cc1] | 1.05 | 369 | 105- 115 |
| 2.16 | 3-[4-(2,6- difluorophenyl)sulfinyl- phenyl]-5-(trifluoromethyl)- 1,2,4-oxadiazole | 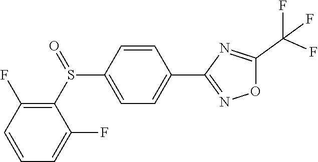 [SMILES: O=S(c1ccc(-c2noc(C(F)(F)F)n2)cc1)c1c(F)cccc1F] | 1.06 | 375 | 148- 155 |
| 2.17 | 3-[4-[(2- fluorophenyl)sulfinylmethyl] phenyl]-5- (trifluoromethyl)-1,2,4- oxadiazole | 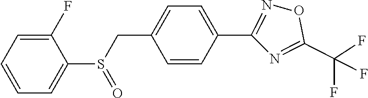 [SMILES: O=S(Cc1ccc(-c2noc(C(F)(F)F)n2)cc1)c1ccccc1F] | 1.12 | 371 | 108- 118 |
| 2.18 | 3-[4-(2- fluorophenyl)sulfinylphenyl]- 5-(trifluoromethyl)-1,2,4- oxadiazole | 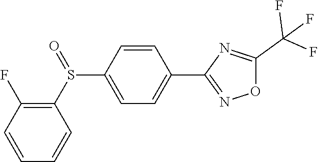 [SMILES: O=S(c1ccc(-c2noc(C(F)(F)F)n2)cc1)c1ccccc1F] | 1.12 | 357 | 116- 121 |
| 2.19 | 3-(4-tert- butylsulfinylphenyl)-5- (trifluoromethyl)-1,2,4- oxadiazole | 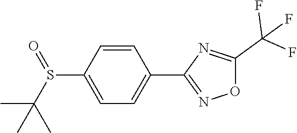 [SMILES: CC(C)(C)S(=O)c1ccc(-c2noc(C(F)(F)F)n2)cc1] | 1.06 | 319 | |
| 2.20 | 3-[4-(3- fluorophenyl)sulfinylphenyl]- 5-(trifluoromethyl)-1,2,4- oxadiazole | 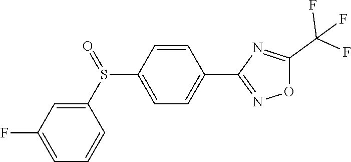 [SMILES: O=S(c1ccc(-c2noc(C(F)(F)F)n2)cc1)c1cccc(F)c1] | 1.05 | 357 | 103- 111 |
| 2.21 | 5-(trifluoromethyl)-3-[4-[2- (trifluoromethyl)phenyl] sulfinylphenyl]-1,2,4- oxadiazole | 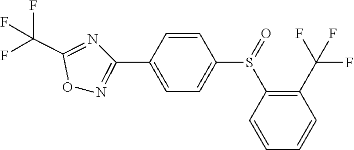 [SMILES: O=S(c1ccc(-c2noc(C(F)(F)F)n2)cc1)c1ccccc1C(F)(F)F] | 1.18 | 407 | 80-86 |
| 2.22 | 3-[4-(2- methylbutylsulfinyl)phenyl]- 5-(trifluoromethyl)-1,2,4- oxadiazole | 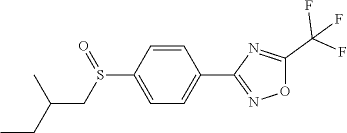 [SMILES: CCC(C)CS(=O)c1ccc(-c2noc(C(F)(F)F)n2)cc1] | 1.08 | 333 | 38-43 |
| 2.23 | 3-(4- cyclopentylsulfinylphenyl)- 5-(trifluoromethyl)-1,2,4- oxadiazole | 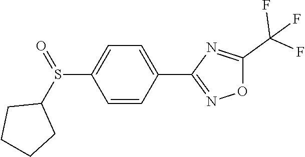 [SMILES: O=S(c1ccc(-c2noc(C(F)(F)F)n2)cc1)C1CCCC1] | 1.04 | 331 | |
| 2.24 | 3-[4-(o- tolylsulfinyl)phenyl]-5- (trifluoromethyl)-1,2,4- oxadiazole | 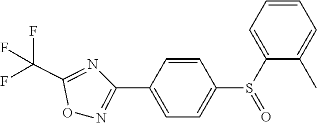 [SMILES: Cc1ccccc1S(=O)c1ccc(-c2noc(C(F)(F)F)n2)cc1] | 1.07 | 353 | 100- 103 |
| 2.25 | 3-[6-(o-tolylsulfinyl)-3- pyridyl]-5-(trifluoromethyl)- 1,2,4-oxadiazole | 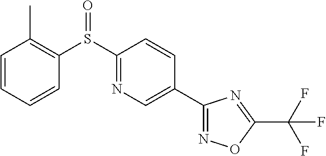 [SMILES: Cc1ccccc1S(=O)c1ccc(-c2noc(C(F)(F)F)n2)cn1] | 1.11 | 354 | 103- 105 |
| 2.26 | 3-[6-(2- fluorophenyl)sulfinyl-3- pyridyl]-5-(trifluoromethyl)- 1,2,4-oxadiazole | 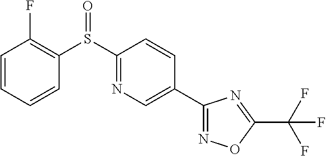 [SMILES: O=S(c1ccc(-c2noc(C(F)(F)F)n2)cn1)c1ccccc1F] | 1.06 | 358 | 92-95 |
| 2.27 | 3-(6-benzylsulfinyl-3- pyridyl)-5-(trifluoromethyl)- 1,2,4-oxadiazole | 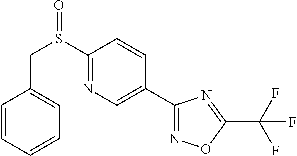 [SMILES: O=S(Cc1ccccc1)c1ccc(-c2noc(C(F)(F)F)n2)cn1] | 1.06 | 354 | 122- 125 |
| 2.28 | 3-[6- (benzenesulfinylmethyl)-3- pyridyl]-5-(trifluoromethyl)- 1,2,4-oxadiazole | 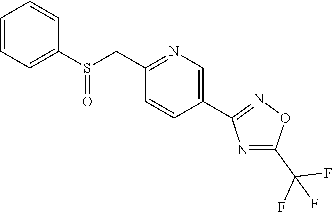 [SMILES: O=S(Cc1ccc(-c2noc(C(F)(F)F)n2)cn1)c1ccccc1] | 0.93 | 354 (M + 1) | 116- 120 |
| 2.29 | 3-[4- (benzenesulfinylmethyl)-3- fluoro-phenyl]-5- (trifluoromethyl)-1,2,4- oxadiazole | 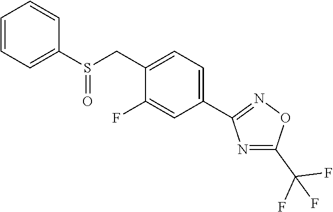 [SMILES: O=S(Cc1ccc(-c2noc(C(F)(F)F)n2)cc1F)c1ccccc1] | 1.06 | 371 (M + 1) | 116- 120 |
| 2.30 | 3-[4- (benzenesulfinylmethyl)-2- fluoro-phenyl]-5- (trifluoromethyl)-1,2,4- oxadiazole | 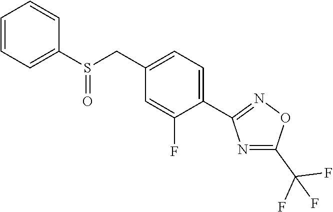 [SMILES: O=S(Cc1ccc(-c2noc(C(F)(F)F)n2)c(F)c1)c1ccccc1] | 1.12 | 371 (M + 1) | 149- 156 |
| 2.31 | 3-[4- (benzenesulfinylmethyl)- 2,3-difluoro-phenyl]-5- (trifluoromethyl)-1,2,4- oxadiazole | 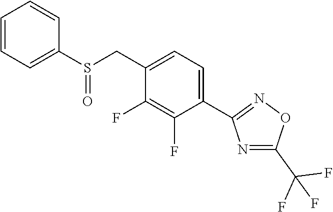 [SMILES: O=S(Cc1ccc(-c2noc(C(F)(F)F)n2)c(F)c1F)c1ccccc1] | 1.05 | 389 (M + 1) | 163- 167 |
| 2.32 | 3-[4- (benzenesulfinylmethyl)- 3,5-difluoro-phenyl]-5- (trifluoromethyl)-1,2,4- oxadiazole | 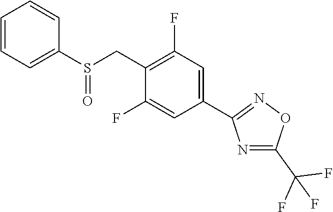 [SMILES: O=S(Cc1c(F)cc(-c2noc(C(F)(F)F)n2)cc1F)c1ccccc1] | 1.08 | 387 (M − 1) | 120- 125 |

TABLE T3
Melting point (mp) data and/or retention times (RT) for the compounds of Formula (I).

| | | | RT | [M + Z] | Mp |
|---|---|---|---|---|---|
| Entry | Compound name | Structure | (min) | (measured) | (° C.) |
| 3.1 | 3-[4- (benzenesulfonyl)phenyl]-5- (trifluoromethyl)-1,2,4- oxadiazole | 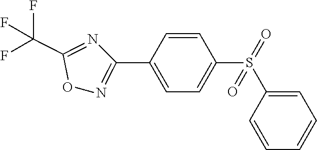 [SMILES: O=S(=O)(c1ccccc1)c1ccc(-c2noc(C(F)(F)F)n2)cc1] | 1.08 | 355 (M + 1) | 128- 132 |
| 3.2 | 3-[4-(4- methoxyphenyl)sulfonyl- phenyl]-5-(trifluoromethyl)- 1,2,4-oxadiazole | 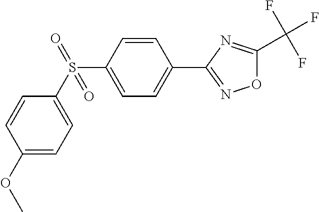 [SMILES: COc1ccc(S(=O)(=O)c2ccc(-c3noc(C(F)(F)F)n3)cc2)cc1] | 1.09 | 385 (M + 1) | 108- 116 |
| 3.3 | 3-(4- cyclohexylsulfonylphenyl)-5- (trifluoromethyl)-1,2,4- oxadiazole | 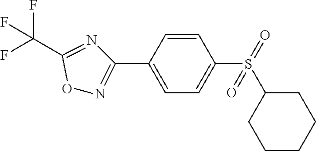 [SMILES: O=S(=O)(c1ccc(-c2noc(C(F)(F)F)n2)cc1)C1CCCCC1] | 1.13 | 361 (M + 1) | 110- 116 |
| 3.4 | 3-[4-(2,4- difluorophenyl)sulfonyl- phenyl]-5-(trifluoromethyl)- 1,2,4-oxadiazole | 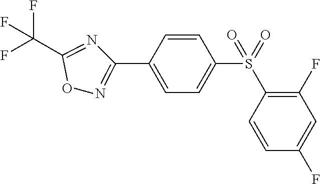 [SMILES: O=S(=O)(c1ccc(-c2noc(C(F)(F)F)n2)cc1)c1ccc(F)cc1F] | 1.11 | 410 (M + 18) | 135- 140 |
| 3.5 | 3-[4-(2,2,2- trifluoroethylsulfonyl)phenyl]- 5-(trifluoromethyl)-1,2,4- oxadiazole | 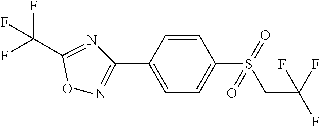 [SMILES: O=S(=O)(CC(F)(F)F)c1ccc(-c2noc(C(F)(F)F)n2)cc1] | 1.04 | mass not detected | 117- 122 |
| 3.6 | 3-[4-(2,5- dimethylphenyl)sulfonyl- phenyl]-5-(trifluoromethyl)- 1,2,4-oxadiazole | 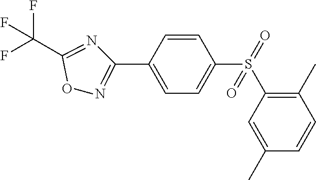 [SMILES: Cc1ccc(C)c(S(=O)(=O)c2ccc(-c3noc(C(F)(F)F)n3)cc2)c1] | 1.17 | 383 (M + 1) | 118- 125 |
| 3.7 | 3-[6-(benzenesulfonyl)-3- pyridyl]-5-(trifluoromethyl)- 1,2,4-oxadiazole | 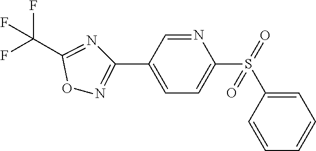 [SMILES: O=S(=O)(c1ccccc1)c1ccc(-c2noc(C(F)(F)F)n2)cn1] | 1.01 | 356 (M + 1) | 124- 129 |
| 3.8 | 3-[4-(benzenesulfonyl)-3- methyl-phenyl]-5- (trifluoromethyl)-1,2,4- oxadiazole | 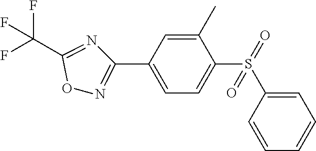 [SMILES: Cc1cc(-c2noc(C(F)(F)F)n2)ccc1S(=O)(=O)c1ccccc1] | 1.13 | 369 (M + 1) | 117- 124 |
| 3.9 | 3-[4-(4- fluorophenyl)sulfonylphenyl]- 5-(trifluoromethyl)-1,2,4- oxadiazole | 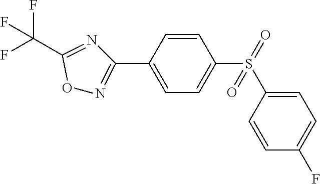 [SMILES: O=S(=O)(c1ccc(F)cc1)c1ccc(-c2noc(C(F)(F)F)n2)cc1] | 1.10 | mass not detected | 144- 149 |
| 3.10 | 3-[4-(2- methoxyphenyl)sulfonyl- phenyl]-5-(trifluoromethyl)- 1,2,4-oxadiazole | 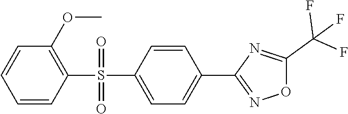 [SMILES: COc1ccccc1S(=O)(=O)c1ccc(-c2noc(C(F)(F)F)n2)cc1] | 1.07 | 385 (M + 1) | 119- 123 |
| 3.11 | 3-[4-[(2- fluorophenyl)sulfonylmethyl] phenyl]-5-(trifluoromethyl)- 1,2,4-oxadiazole | 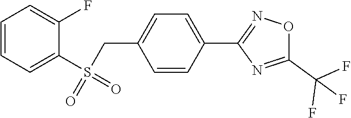 [SMILES: O=S(=O)(Cc1ccc(-c2noc(C(F)(F)F)n2)cc1)c1ccccc1F] | 1.11 | mass not detected | 150- 154 |
| 3.12 | 3-[4-(2- fluorophenyl)sulfonylphenyl]- 5-(trifluoromethyl)-1,2,4- oxadiazole | 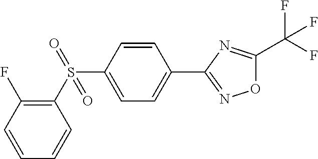 [SMILES: O=S(=O)(c1ccc(-c2noc(C(F)(F)F)n2)cc1)c1ccccc1F] | 1.14 | 373 (M + 1) | 140- 147 |
| 3.13 | 3-(4-tert- butylsulfonylphenyl)-5- (trifluoromethyl)-1,2,4- oxadiazole | 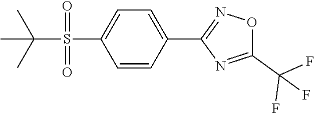 [SMILES: CC(C)(C)S(=O)(=O)c1ccc(-c2noc(C(F)(F)F)n2)cc1] | 1.11 | 335 (M + 1) | 133- 136 |
| 3.14 | 3-[4-(3- fluorophenyl)sulfonylphenyl]- 5-(trifluoromethyl)-1,2,4- oxadiazole | 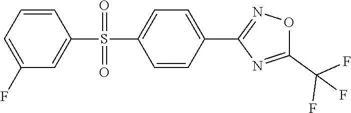 [SMILES: O=S(=O)(c1ccc(-c2noc(C(F)(F)F)n2)cc1)c1cccc(F)c1] | 1.1 | 390 (M + 18) | 136- 143 |
| 3.15 | 3-[4-(2- methylbutylsulfonyl)phenyl]- 5-(trifluoromethyl)-1,2,4- oxadiazole | 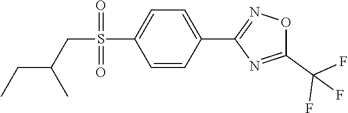 [SMILES: CCC(C)CS(=O)(=O)c1ccc(-c2noc(C(F)(F)F)n2)cc1] | 1.12 | 366 (M + 18) | 75-80 |
| 3.16 | 3-(4- cyclopentylsulfonylphenyl)- 5-(trifluoromethyl)-1,2,4- oxadiazole | 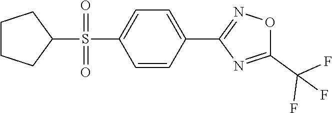 [SMILES: O=S(=O)(c1ccc(-c2noc(C(F)(F)F)n2)cc1)C1CCCC1] | 1.08 | 364 (M + 18) | 85-92 |
| 3.17 | 3-[4-(o-tolylsulfonyl)phenyl]- 5-(trifluoromethyl)-1,2,4- oxadiazole | 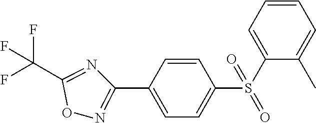 [SMILES: Cc1ccccc1S(=O)(=O)c1ccc(-c2noc(C(F)(F)F)n2)cc1] | 1.12 | 369 (M + 1) | 109- 115 |
| 3.18 | 3-[6-(o-tolylsulfonyl)-3- pyridyl]-5-(trifluoromethyl)- 1,2,4-oxadiazole | 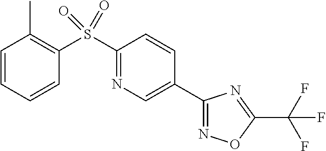 [SMILES: Cc1ccccc1S(=O)(=O)c1ccc(-c2noc(C(F)(F)F)n2)cn1] | 1.13 | 387 (M + 18) | 66-68 |
| 3.19 | 3-[6-(2- fluorophenyl)sulfonyl-3- pyridyl]-5-(trifluoromethyl)- 1,2,4-oxadiazole | 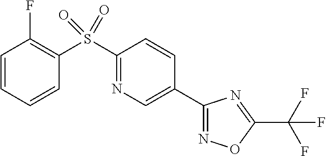 [SMILES: O=S(=O)(c1ccc(-c2noc(C(F)(F)F)n2)cn1)c1ccccc1F] | 1.07 | 374 (M + 1) | 145- 151 |
| 3.20 | 3-[4-(2- chlorophenyl)sulfonylphenyl]- 5-(trifluoromethyl)-1,2,4- oxadiazole | 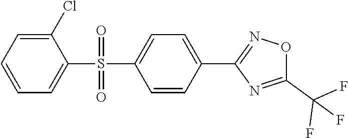 [SMILES: O=S(=O)(c1ccc(-c2noc(C(F)(F)F)n2)cc1)c1ccccc1Cl] | 1.13 | 405 (M + 17) | 127- 135 |
| 3.21 | 3-[6- (benzenesulfonylmethyl)-3- pyridyl]-5-(trifluoromethyl)- 1,2,4-oxadiazole | 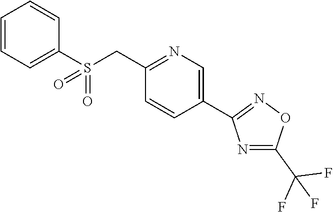 [SMILES: O=S(=O)(Cc1ccc(-c2noc(C(F)(F)F)n2)cn1)c1ccccc1] | 0.98 | 370 (M − 1) | 160- 166 |
| 3.22 | 3-[4- (benzenesulfonylmethyl)-2- fluoro-phenyl]-5- (trifluoromethyl)-1,2,4- oxadiazole | 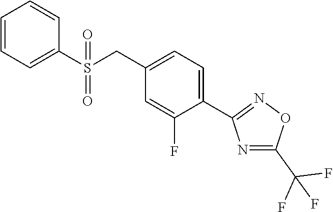 [SMILES: O=S(=O)(Cc1ccc(-c2noc(C(F)(F)F)n2)c(F)c1)c1ccccc1] | 1.14 | 385 (M − 1) | 172- 178 |
| 3.23 | 3-[4- (benzenesulfonylmethyl)- 2,3-difluoro-phenyl]-5- (trifluoromethyl)-1,2,4- oxadiazole | 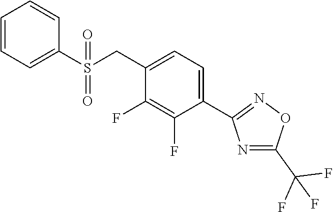 [SMILES: O=S(=O)(Cc1ccc(-c2noc(C(F)(F)F)n2)c(F)c1F)c1ccccc1] | 1.08 | 403 (M − 1) | 187- 191 |
| 3.24 | 3-[4- (benzenesulfonylmethyl)- 3,5-difluoro-phenyl]-5- (trifluoromethyl)-1,2,4- oxadiazole | 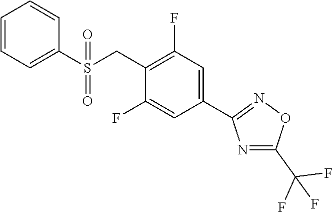 [SMILES: O=S(=O)(Cc1c(F)cc(-c2noc(C(F)(F)F)n2)cc1F)c1ccccc1] | 1.1 | 403 (M − 1) | 135- 140 |

### Biological Examples

General Examples of Leaf Disk Tests in Well Plates:

Leaf disks or leaf segments of various plant species are cut from plants grown in a greenhouse. The cut leaf disks or segments are placed in multiwell plates (24-well format) onto water agar. The leaf disks are sprayed with a test solution before (preventative) or after (curative) inoculation. Compounds to be tested are prepared as DMSO solutions (max. 10 mg/ml) which are diluted to the appropriate concentration with 0.025% Tween20 just before spraying. The inoculated leaf disks or segments are incubated under defined conditions (temperature, relative humidity, light, etc.) according to the respective test system. A single evaluation of disease level is carried out 3 to 14 days after inoculation, depending on the pathosystem. Percent disease control relative to the untreated check leaf disks or segments is then calculated.

General Examples of Liquid Culture Tests in Well Plates:

Mycelia fragments or conidia suspensions of a fungus prepared either freshly from liquid cultures of the fungus or from cryogenic storage, are directly mixed into nutrient broth. DMSO solutions of the test compound (max. 10 mg/ml) are diluted with 0.025% Tween20 by a factor of 50 and 10 μl of this solution is pipetted into a microtiter plate (96-well format). The nutrient broth containing the fungal spores/mycelia fragments is then added to give an end concentration of the tested compound. The test plates are incubated in the dark at 24° C. and 96% relative humidity. The inhibition of fungal growth is determined photometrically after 2 to 7 days, depending on the pathosystem, and percent antifungal activity relative to the untreated check is calculated.

### Example 1: Fungicidal Activity Against Puccinia recondita f. Sp. Tritici/Wheat/Leaf Disc Preventative (Brown Rust)

Wheat leaf segments cv. Kanzler were placed on agar in multiwell plates (24-well format) and sprayed with the formulated test compound diluted in water. The leaf disks were inoculated with a spore suspension of the fungus 1 day after application. The inoculated leaf segments were incubated at 19° C. and 75% relative humidity (rh) under a light regime of 12 hours light/12 hours darkness in a climate cabinet and the activity of a compound was assessed as percent disease control compared to untreated when an appropriate level of disease damage appears in untreated check leaf segments (7 to 9 days after application).

The following compounds at 200 ppm in the applied formulation give at least 80% disease control in this test when compared to untreated control leaf disks under the same conditions, which show extensive disease development.

Compounds (from Table T1) 1.1, 1.2, 1.5, 1.14, 1.23, and 1.26.

Compounds (from Table T2) 2.1, 2.2, 2.3, 2.5, 2.6, 2.7, 2.8, 2.10, 2.11, 2.12, 2.13, 2.14, 2.15, 2.16, 2.18, 2.19, 2.20, 2.23, 2.24, 2.25, 2.26, 2.28, and 2.31.

Compounds (from Table T3) 3.1, 3.2, 3.7, 3.9, 3.10, 3.14, 3.16, 3.17, 3.18, and 3.19.

### Example 2: Fungicidal Activity Against Puccinia recondita f. Sp. Tritici/Wheat/Leaf Disc Curative (Brown Rust)

Wheat leaf segments cv. Kanzler are placed on agar in multiwell plates (24-well format). The leaf segments are then inoculated with a spore suspension of the fungus. Plates were stored in darkness at 19° C. and 75% relative humidity. The formulated test compound diluted in water was applied 1 day after inoculation. The leaf segments were incubated at 19° C. and 75% relative humidity under a light regime of 12 hours light/12 hours darkness in a climate cabinet and the activity of a compound was assessed as percent disease control compared to untreated when an appropriate level of disease damage appears in untreated check leaf segments (6 to 8 days after application).

The following compounds at 200 ppm in the applied formulation give at least 80% disease control in this test when compared to untreated control leaf disks under the same conditions, which show extensive disease development.

Compounds (from Table T1) 1.1, 1.2, 1.5, 1.7, 1.12, 1.14, 1.15, 1.23, and 1.26.

Compounds (from Table T2) 2.1, 2.2, 2.3, 2.5, 2.6, 2.8, 2.10, 2.12, 2.13, 2.15, 2.19, 2.22, 2.23, 2.24, and 2.26, 2.28, and 2.32.

Compounds (from Table T3) 3.1 and 3.16.

### Example 3: Fungicidal Activity Against Phakopsora pachyrhizi/Soybean/Leaf Disc Preventative (Asian Soybean Rust)

Soybean leaf disks are placed on water agar in multiwell plates (24-well format) and sprayed with the formulated test compound diluted in water. One day after application leaf discs are inoculated by spraying a spore suspension on the lower leaf surface. After an incubation period in a climate cabinet of 24-36 hours in darkness at 20° C. and 75% rh leaf disc are kept at 20° C. with 12 h light/day and 75% rh. The activity of a compound is assessed as percent disease control compared to untreated when an appropriate level of disease damage appears in untreated check leaf disks (12 to 14 days after application).

The following compounds at 200 ppm in the applied formulation give at least 80% disease control in this test when compared to untreated control leaf disks under the same conditions, which show extensive disease development.

Compounds (from Table T1) 1.1, 1.2, 1.4, 1.5, 1.6, 1.7, 1.9, 1.10, 1.11, 1.12, 1.13, 1.14, 1.15, 1.17, 1.18, and 1.20, 1.24, 1.25, and 1.26.

Compounds (from Table T2) 2.1, 2.2, 2.3, 2.5, 2.6, 2.7, 2.8, 2.9, 2.10, 2.11, 2.12, 2.13, 2.15, 2.16, 2.17, 2.18, 2.19, 2.20, 2.21, 2.22, 2.23, 2.25, 2.28, 2.29, 2.20, 2.31, and 2.32.

Compounds (from Table T3) 3.1, 3.2, 3.3, 3.4, 3.7, 3.9, 3.11, 3.12, 3.14, 3.16, 3.20, 3.21, 3.22, 3.23, and 3.24.

### Example 4: Fungicidal Activity Against Glomerella lagenarium (Colletotrichum lagenarium) Liquid Culture/Cucumber/Preventative (Anthracnose)

Conidia of the fungus from cryogenic storage are directly mixed into nutrient broth (PDB—potato dextrose broth). After placing a (DMSO) solution of test compound into a microtiter plate (96-well format), the nutrient broth containing the fungal spores is added. The test plates are incubated at 24° C. and the inhibition of growth is measured photometrically 3 to 4 days after application.

The following compounds at 20 ppm in the applied formulation give at least 80% disease control in this test when compared to untreated control under the same conditions, which show extensive disease development.

Compounds (from Table T1) 1.1, 1.2, 1.5, 1.7, 1.10, 1.12, 1.16, 1.19, 1.20, and 1.21, 1.23, 1.24, 1.25, 1.26, and 1.27.

Compounds (from Table T2) 2.1, 2.2, 2.3, 2.4, 2.5, 2.6, 2.7, 2.8, 2.9, 2.10, 2.11, 2.12, 2.14, 2.17, 2.18, 2.19, 2.20, 2.22, 2.23, 2.24, 2.25, 2.26, 2.27, 2.28, 2.29, 2.31, and 2.32.

Compounds (from Table T3) 3.1, 3.2, 3.3, 3.4, 3.5, 3.6, 3.7, 3.8, 3.12, 3.13, 3.14, 3.15, 3.16, 3.17, 3.18, 3.19, 3.20, and 3.21.

1. A compound of formula (I): 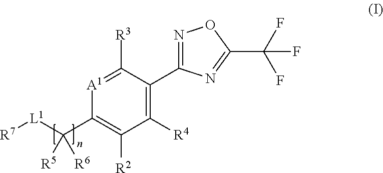
wherein
A¹ represents N or CR¹, wherein R¹ is selected from hydrogen, halogen, methyl, trifluoromethyl or methoxy;
R² is hydrogen or halogen;
R³ and R⁴ are independently selected from hydrogen and fluorine; and
wherein at least two of R¹ to R⁴ are hydrogen;
n represents 0, 1 or 2;
R⁵ and R⁶ are independently selected from hydrogen, C₁₋₄alkyl and cyano;
L¹ represents S, S(O) or S(O)₂;
R⁷ represents hydrogen, C₁₋₆alkyl, C₂₋₆alkenyl, C₂₋₆alkynyl, cyanoC₁₋₆alkyl, C₁₋₆haloalkyl, C₂₋₆haloalkenyl, hydroxyC₁₋₆alkyl, C₁₋₄alkoxyC₁₋₆alkyl, C₁₋₄alkoxyC₁₋₆alkoxy or C₁₋₄haloalkoxyC₁₋₆alkyl; or
R⁷ represents C₃₋₈cycloalkyl or C₃₋₈cycloalkylC₁₋₃alkyl wherein the cycloalkyl moiety is optionally partially unsaturated, phenyl, phenylC₁₋₃alkyl, heteroaryl bonded to L¹ through a carbon atom or heteroarylC₁₋₃alkyl wherein the heteroaryl moiety is a 5- or 6-membered monocyclic aromatic ring which comprises 1, 2, 3 or 4 heteroatoms individually selected from N, O and S, heterocyclyl bonded to L¹ through a carbon atom or heterocyclylC₁₋₃alkyl wherein the heterocyclyl moiety is a 4- to 6-membered non-aromatic ring which comprises 1, 2 or 3 heteroatoms individually selected from N, O and S, and wherein C₃₋₈cycloalkyl, C₃₋₈ cycloalkylC₁₋₃alkyl, phenyl, phenylC₁₋₃alkyl, heteroaryl, heteroarylC₁₋₃alkyl, heterocyclyl and heterocyclylC₁₋₃alkyl are optionally substituted by 1, 2, 3, 4 or 5 substituents, which may be the same or different, selected from R⁸;
R⁸ represents cyano, halogen, hydroxy, C₁₋₄alkyl, C₁₋₄haloalkyl, C₁₋₄alkoxy or C₁₋₄haloalkoxy; and
wherein when R⁷ represents C₃₋₈cycloalkyl, C₃₋₈cycloalkylC₁₋₃alkyl, heterocyclyl or heterocyclylC₁₋₃alkyl, the C₃₋₈cycloalkyl moiety or the heterocyclyl moiety is optionally substituted by 1 or 2 oxo groups; or
a salt or an N-oxide thereof.

2. A compound according to claim 1, wherein L¹ is S or S(O).

3. A compound according to claim 1, wherein A¹ represents N or CR¹, wherein R¹ is selected from hydrogen or methyl.

4. A compound according to claim 1, wherein R², R³ and R⁴ are independently selected from hydrogen and fluorine.

5. A compound according to claim 1, wherein R², R³ and R⁴ are hydrogen.

6. A compound according to claim 1, wherein n is 0, or n is 1 and R⁵ and R⁶ are hydrogen or R⁵ is hydrogen and R⁶ is methyl.

7. A compound according to claim 1, wherein n is 0.

8. A compound according to claim 1, wherein:
R⁷ is hydrogen, C₁₋₆alkyl, C₂₋₆alkenyl, C₂₋₆alkynyl, cyanoC₁₋₆alkyl, C₁₋₆haloalkyl or C₁₋₄ alkoxyC₁₋₆alkyl; or
R⁷ is C₃₋₈cycloalkyl or C₃₋₈cycloalkylC₁₋₂alkyl wherein the cycloalkyl moiety is optionally partially unsaturated, phenyl or phenylC₁₋₂alkyl, heteroaryl bonded to L¹ through a carbon atom or heteroarylC₁₋₂alkyl wherein the heteroaryl moiety is a 5- or 6-membered monocyclic aromatic ring which comprises 1, 2, 3 or 4 heteroatoms individually selected from N, O and S, or heterocyclyl bonded to L¹ through a carbon atom or heterocyclylC₁₋₂alkyl wherein the heterocyclyl moiety is a 4- to 6-membered non-aromatic ring which comprises 1, 2 or 3 heteroatoms individually selected from N, O and S; wherein
any C₃₋₈cycloalkyl or C₃₋₈cycloalkylC₁₋₂alkyl, phenyl or phenylC₁₋₂alkyl, heteroaryl or heteroarylC₁₋₂alkyl, or heterocyclyl or heterocyclylC₁₋₂alkyl moiety is optionally substituted by 1, 2, or 3 substituents, which may be the same or different, selected from R⁸; wherein R⁸ represents halogen, C₁₋₄alkyl, C₁₋₄haloalkyl and C₁₋₄alkoxy.

9. A compound according to claim 1, wherein R⁷ is C₁₋₆alkyl, C₂₋₆alkenyl, C₂₋₆alkynyl, C₁₋₆haloalkyl or C₁₋₄alkoxyC₁₋₆alkyl; or
R⁷ is C₃₋₈cycloalkyl, C₃₋₈cycloalkylmethyl, phenyl or phenylC₁₋₂alkyl, wherein any C₃₋₈cycloalkyl and phenyl moiety is optionally substituted by 1, 2 or 3 substituents, which may be the same or different, selected from R⁸, wherein R⁸ is halogen, C₁₋₄alkyl, C₁₋₄haloalkyl and C₁₋₄alkoxy.

10. A compound according to claim 1, wherein R⁷ is C₁₋₆alkyl, C₁₋₆haloalkyl, C₃₋₈cycloalkyl, phenyl or phenylC₁₋₂alkyl, wherein C₃₋₈cycloalkyl or any phenyl moiety is optionally substituted by 1 or 2 substituents, which may be the same or different, selected from R⁸, wherein R⁸ is halogen, C₁₋₄alkyl, C₁₋₄haloalkyl and C₁₋₄alkoxy.

11. A compound according to claim 1, wherein R⁷ is C₁₋₄alkyl, C₁₋₃fluoroalkyl, C₅₋₆cycloalkyl, phenyl or phenylC₁₋₂alkyl, wherein C₅₋₆cycloalkyl or any phenyl moiety is optionally substituted with 1 or 2 substituents, which may be the same or different, selected from R⁸, wherein R⁸ is fluorine, chlorine, methyl, ethyl, difluoromethyl, trifluoromethyl, methoxy and ethoxy.

12. An agrochemical composition comprising a fungicidally effective amount of a compound of formula (I) according to claim 1.

13. A composition according to claim 12, further comprising at least one additional active ingredient and/or an agrochemically-acceptable diluent or carrier.

14. A method of controlling or preventing infestation of useful plants by phytopathogenic microorganisms, wherein a fungicidally effective amount of a compound of formula (I) according to claim 1, or a composition comprising this compound as active ingredient, is applied to the plants, to parts thereof or the locus thereof.

15. Use of a compound of formula (I) according to claim 1 as a fungicide.
